(19) United States
(12) Patent Application Publication
(10) Pub. No.: US 2024/0161001 A1
(43) Pub. Date: May 16, 2024

(54) DATA PROCESSING PIPELINE AND PLATFORM FOR ZEBRAFISH PHENOTYPING

(71) Applicant: BioSymetrics, Inc., New York, NY (US)

(72) Inventors: Kevin C.H. Ha, New York, NY (US); Rokshana Stephny Geread, New York, NY (US); Steven Bishop, New York, NY (US); Nishanth Merwin, New York, NY (US); Jonathan R. Volpatti, New York, NY (US); David Kokel, New York, NY (US); Mikalai Malinouski, New York, NY (US); Gabriel Musso, New York, NY (US)

(73) Assignee: BioSymetrics, Inc., New York, NY (US)

(21) Appl. No.: 18/150,168

(22) Filed: Jan. 4, 2023

Related U.S. Application Data
(60) Provisional application No. 63/383,749, filed on Nov. 15, 2022.

(51) Int. Cl. G06N 20/00; G06N 3/045; G06N 3/08; G06V 10/774

(52) U.S. Cl. CPC G06N 20/00; G06N 3/045; G06N 3/08; G06V 10/774

(57) ABSTRACT

Embodiments are directed to a platform for zebrafish phenotyping that includes a user, images and videos containing zebrafish and elements pertaining to the zebrafish (such as organs, organ systems, and tissues), a first set of machine learning models used to predict zebrafish phenotypes based on the image and video data, and a second set of machine learning models used to automatically detect elements in one of the images and videos containing zebrafish. This platform may be used to detect changes in phenotypes and may be used to detect alteration is specific organs in zebrafish.

### BACKGROUND

The present disclosure relates to methods and apparatus for acquiring, analyzing, managing, and applying data and findings concerning zebrafish phenotyping. Some of these embodiments more specifically relate to methods and apparatus for experimental design, data acquisition, data analysis, and data management of zebrafish phenotyping used to validate predictions made using machine learning models, as well as to generate other phenotypic data that may be used for novel drug discovery and application.

### SUMMARY

Zebrafish are a popular model organism in developmental biology, molecular genetics, and toxicology studies due to their small size, low breeding costs, transparent embryos, morphological identification, reproduction rate, fast development time, and similarity of their genome to the human genome. They are generally used to study the toxic effects of various drugs on cell apoptosis, organ development, cardiac and nervous system functions, teratogenic assays, and genetics relating to human orthologs and orthologous groups. Zebrafish are also considered a suitable model for studies relating to human disease due to their ability to process large numbers of samples, provide gradual development, and their relation to human cellular mechanisms.

Zebrafish orthologs are a group of genes that are homologous between zebrafish (Danio rerio) and other species. Orthologs are genes that are similar between two species and are the result of shared ancestry. Zebrafish are a popular model organism for biological research due to their small size, short life cycle, and availability of genomic information. Zebrafish orthologs are widely studied to understand the genetic basis of specific phenotypes, such as embryonic development and disease. For example, genes known to be involved in human diseases can be studied in zebrafish to understand the genetic basis of the disease in humans. Additionally, zebrafish orthologs can be used to study the evolution of genes and help researchers identify novel genes with potential therapeutic applications. Furthermore, zebrafish orthologs can provide insight into the conservation of gene function between species. By comparing the orthologs between species, researchers can gain a better understanding of how proteins, cell signaling pathways, and transcriptional networks are conserved across species. Zebrafish orthologs also allow for the comparison of gene function across species and can provide insight into the evolution of genes and their potential therapeutic applications.

In addition to studying the effects of genetic disorders, gene testing in zebrafish may also be used to create new therapeutic approaches to treat diseases. For example, researchers may use gene testing to identify genes to be targeted to treat certain diseases. These genes can then be modified to create new therapeutic approaches to treat the disease. This is especially useful for diseases that currently have no known cure or treatment.

Some embodiments may include fluorescent or bright-field imaging of zebrafish to determine alterations in phenotype data. These images may be compared to detect alterations of phenotypes between zebrafish during or after genetic testing.

Some embodiments may concern a zebrafish phenotyping platform that provides the capability to screen both genetic and chemical perturbations in zebrafish and identify disease-relevant phenotypes. This platform may be used to identify zebrafish phenotypes using image and video data. To ensure this platform can perform at scale in an error-free manner, a data flow architecture that allows for efficient data processing and data access for end users is described.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 is a block diagram of an exemplary system for preprocessing biomedical data, in accordance with some embodiments of the present disclosure;

FIGS. 2A-C depict a block diagram of a machine learning (ML) framework, in accordance with some embodiments of the present disclosure;

FIG. 3 is a block diagram of the ML framework of FIGS. 2A-C functioning over a parallel computing network, in accordance with some embodiments of the present disclosure;

FIG. 4 is a block diagram of a preprocessing engine, in accordance with some embodiments of the present disclosure;

FIG. 5 is a flow diagram of an exemplary process for preprocessing biomedical data, in accordance with some embodiments of the present disclosure;

FIG. 6 is a flow diagram of an exemplary process of preprocessing biomedical data using the parallel computing network of FIG. 3, in accordance with some embodiments of the present disclosure;

FIG. 7 is a flow diagram of an exemplary process of merging a plurality of datasets and selecting relevant features from the combined dataset using the parallel computing network of FIG. 3, in accordance with some embodiments of the present disclosure;

FIG. 8 is a block diagram depicting the examples of input sources and operations performed by the parallel computing network of FIG. 3, in accordance with some embodiments of the present disclosure;

FIG. 9 is a block diagram of an exemplary computer system for implementing embodiments consistent with the present disclosure.

FIG. 10 is a flow diagram of an exemplary process of preprocessing parameter optimization included in the ML predictive model creation process.

FIG. 11 is a flow diagram of an exemplary process of integrating heterogeneous datasets for ML predictive model creation.

FIG. 12 is a flow diagram of an exemplary process for using a cloud network for data preprocessing specifically in biomedical predictive model creation.

FIG. 13 is a flow diagram of an exemplary process for using parallel computing networks and associated API specifically for data preprocessing for biomedical predictive model creation over a cloud network.

FIG. 14 is an exemplary bias correction iteration sequence for a single bias correction algorithm to normalize the data.

FIG. 15 is an exemplary iteration sequence of bias correction algorithms.

FIG. 16 is a diagram illustrating a method for Gene Disease Prioritization with use of a phenograph;

FIG. 17 is a diagram illustrating a method for producing human disease to zebrafish phenotype relationships;

FIG. 18 is a diagram illustrating the connection of a feature matrix with an API layer that sends biological data to the phenograph;

FIG. 19 is a diagram illustrating a method for creating and updating the phenograph development environment;

FIG. 20 is a diagram illustrating how the data is collected, harmonized, and stored in an SQLite database;

FIG. 21 is a depiction of the phenograph database schema;

FIG. 22 is a diagram illustrating the phenograph deployment (deployed) environment architecture;

FIG. 23 is a diagram illustrating the phenograph local development environment architecture;

FIG. 24 is an image of the phenograph with the navigation tab, search bar, filters, and other features;

FIG. 25 is an image of the phenograph after the navigation tab is clicked;

FIG. 26 is an image of the “Phenotype ID to Genes” page in the phenograph;

FIG. 27 is an image after searching for “IHP:0000458” (anosmia) in the “Phenotype ID to Genes” search category;

FIG. 28 is an image after searching for “familial dilated” in the “Disease Name to Disease IDs” search category;

FIG. 29 is an image of the “Disease ID to Genes” page in the phenograph

FIG. 30 is a flowchart of the typical drug discovery workflow with the use of the phenograph;

FIG. 31 is a more in-depth diagram of FIG. 16 illustrating the method for model training for the phenograph and gene-disease prioritization machine learning models;

FIG. 32 is a more in-depth diagram of FIG. 16 illustrating the aggregate scoring of genes to phenotypes and diseases, and the production of a gene-disease association score;

FIG. 33 is a diagram illustrating a small molecule screening process for drug development.

FIG. 34 is a diagram illustrating a small molecule screening software and method of screening; and

FIG. 35 is an illustration of the probability result after running the small molecule screening.

FIG. 36 is a diagram illustrating the steps taken by the MoA engine to provide insight on the MoA of certain drug-phenotype interactions

FIG. 37 is a schematic depicting the basic units of the zebrafish phenotyping platform;

FIGS. 38a-38f contains models of stamps used for mounting zebrafish in a single-well or multi-well plate.

FIGS. 39a-39b contains images of zebrafish in multiple orientations

FIG. 40 contains multiple images of the automated segmented labeling created automatically by a machine learning model.

FIG. 41 is a flowchart for the creation of an optical flow pattern and video

FIG. 42 is a flowchart depicting machine learning and training methods used for Automated Segmentation of zebrafish images.

### DETAILED DESCRIPTION

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims.

### (1) Summaries of Various Embodiments

One or more embodiments of preprocessing biomedical data for a predictive model are disclosed. The one or more embodiments provide for an ML framework for analyzing biomedical data using a predictive model. The one or more embodiments make use of the various components including preprocessing, feature selection, data integration, and parallel computing network.

### (1.1) Preprocessing

Preprocessing is a method for preparing data, in its raw form, for further data analysis in a predictive model. Raw data may not be in a suitable format and may also contain biases due to differences in equipment, variations in equipment use, or variations in reporting of data. Data in the form of images, for example, needs to be converted to a matrix form for data analysis. Preprocessing also ensures that data biases do not lead to faulty predictions by detecting and correcting them. Different datasets have different preprocessing requirements and each of the steps of a preprocessing algorithm may have a plurality of parameters.

### (1.2) Feature Selection

Features are independent random variables on which the outcome or the result of the analysis is dependent. In data, a lot of variables may be present. Using all of these in analysis may give misleading results for a predictive model. Feature selection is a process which performs the selection of relevant independent variables so as to enhance the accuracy of the predictive model.

### (1.3) Data Integration

Data integration is the process of combining a plurality of datasets into a single dataset for data analysis. Each of the plurality of datasets may have different preprocessing needs but the combined dataset will have all the features of each of the plurality of datasets. Consequently, it will lead to high accuracy predictions and a reliable predictive model.

### (1.4) Parallel Computing Network

A parallel computing network consists of a plurality of Central Processing Units (CPUs) working in parallel to provide an enhanced computational capability for the computational task allotted to the network. A parallel computing network may also allow multiple users working on a common task, thereby increasing productivity and efficiency of a workplace.

### (2) Exemplary Environments to Employ Various Embodiments

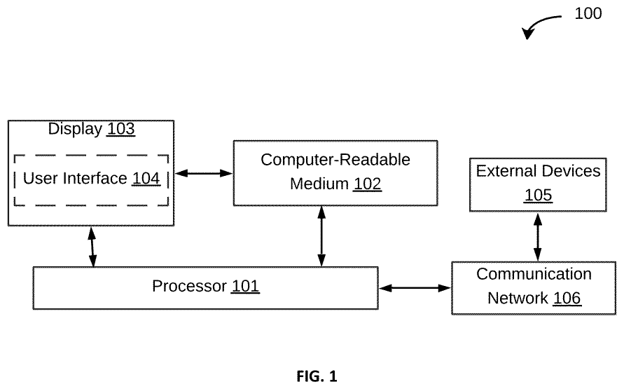

Referring now to FIG. 1, an exemplary system 100 for preprocessing a biomedical data is illustrated, in accordance with some embodiments of the present disclosure. The system 100 may implement a preprocessing engine, in accordance with some embodiments of the present disclosure. In particular, the system 100 may include a preprocessing device (for example, server, desktop, laptop, notebook, netbook, tablet, smartphone, mobile phone, or any other computing device) that may implement the preprocessing engine. The preprocessing engine may preprocess the biomedical data using a machine learning (ML) algorithm.

The system 100 may include one or more processors 101, a computer-readable medium (for example, a memory) 102, and a display 103. The computer-readable storage medium 102 may store instructions that, when executed by the one or more processors 101, cause the one or more processors 101 to preprocess the biomedical data, in accordance with aspects of the present disclosure. The computer-readable storage medium 102 may also store various data that may be captured, processed, and/or required by the system 100. The system 100 may interact with a user via a user interface 104 accessible via the display 103. The system 100 may also interact with one or more external devices 105 over a communication network 106 for sending or receiving various data. The external devices 105 may include, but may not be limited to, a remote server, a digital device, or another computing system.

### (3) Exemplary Systems for Various Embodiments

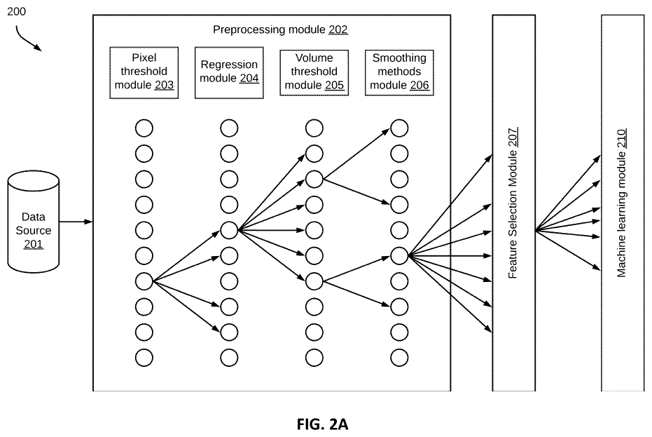

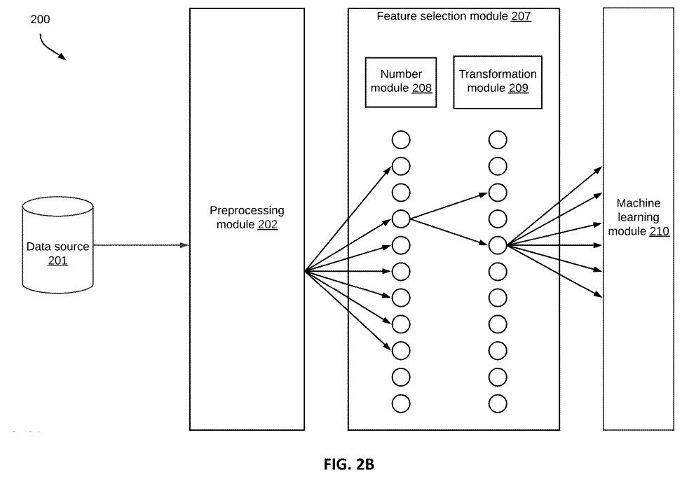

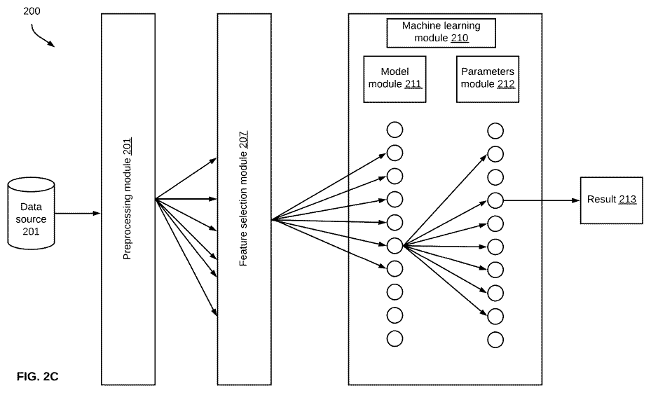

Referring now to FIGS. 2A-C, a block diagram of an ML framework 400 implemented by the system 100, is illustrated, in accordance with some embodiments of the present disclosure. The ML framework 200 includes a data source 201, a preprocessing module 202, a feature selection module 207, and an ML module 210.

The data source 201 is a system for storage of a data and provides an input data to the preprocessing module 202. Some examples include, but may not be limited to, a local storage data, a database, or a cloud storage data. There may be more than one data sources for the ML framework 200.

The preprocessing module 202 includes a pixel threshold module 203, a regression module 204, a volume threshold module 205, and a smoothing methods module 206. The preprocessing module 202 receives the input data and returns a preprocessed input data as an output.

The pixel threshold module 203 uses a pixel thresholding algorithm on the input data, wherein the input data is an image. The pixel thresholding algorithm simplifies the input data for analytical purposes. The parameters for a pixel thresholding algorithm may be an intensity of each of pixels of an image or a color of each of the pixels of the image.

The regression module 204 uses a regression algorithm to perform a preprocessing of the input data. The regression algorithm may be a linear or a non-linear regression algorithm. The preprocessing of the input data may be in the form of a transformation of the input data, a reduction in the outliers of the input data, a thresholding of the input data, a normalization of the input data, any other conventional preprocessing techniques, or any preprocessing technique yet to be discovered.

The volume threshold module 205 uses a volume thresholding algorithm on the input data, wherein the input data is a 3-dimensional (3D) image such as MRI or CT scan. The volume thresholding algorithm simplifies the input data for a volumetric analysis, wherein the volumetric analysis may be used for estimating a volume of a region (for example, a hypothalamus region of a human brain in an MRI image) from the 3D image. The parameters for a volume thresholding algorithm may include a threshold for reduction of noise in the input data and a 3-dimensional region to be analyzed.

The smoothing methods module 206 uses at least one smoothing method to simplify and generalize the input data. The smoothing methods may include, but may not be limited to, an additive smoothing algorithm, an exponential smoothing algorithm, a kernel smoother, a Laplacian smoothing algorithm, and any other data smoothing or data filtering algorithm. The use of a particular smoothing method depends on the type and distribution of the input data.

The feature selection module 207 includes a number module 208 and a transformation module 209. The feature selection module 207 receives an input data from the preprocessing module 202 and returns a set of features relevant for the predictive analysis of the predictive model.

The number module 208 generates a number of features to be used for the predictive analysis of the input data, wherein a feature is a random variable having an impact on an outcome of the predictive model. The feature selection module 207 may iterate over a range of two given numbers of features to select a suitable number of features for the predictive model.

Once the number of features is generated, the transformation module 209 then uses a transformation algorithm such as a principal component analysis (PCA), independent component analysis (ICA), or any other linear or non-linear feature transformation algorithms. The transformation algorithm converts the selected features into different functions of the selected features. A linear transformation algorithm maintains the linear relationships of a feature with other features whereas a nonlinear transformation algorithm changes the linear relationships of a feature with other features. The transformation module 209 may iterate over different transformation algorithms and their associated parameters to select a suitable transformation algorithm and a suitable set of associated parameters for the predictive model.

The ML module 210 includes a model module 211 and a parameters module 212. The ML module 210 uses an ML algorithm to perform a predictive analysis using the preprocessed data obtained from the preprocessing module 202 and the features obtained from the feature selection module 207. The predictive analysis may be, but may not be limited to, diagnosis of a disease, prediction of a probability of getting a disease, and determining an optimum treatment course for a more personalized and high precision medicine course. The ML module 210 gives a result 213 as an output. The result 213 includes the predictions of the ML framework 200 based on the input data received from the data source 201. The result 213 may be visualized using any of the standard data visualization packages such as Seaborn or Matplotlib.

The model module 211 selects a suitable predictive model, based on the data type of the input data, for performing the predictive analysis using the input data. The suitable predictive model may be a support vector machine (SVM) model, a random forest (RF) model, a neural network (NN) model, or any other ML model or a deep learning model, or a combination thereof. The model module 211 receives the preprocessed data (from the preprocessing module 202) and the features (from the feature selection module 207) as an input and generates the suitable predictive model for predictive analysis. In another embodiment, the suitable predictive model may be generated as a result of iterations performed by a second ML algorithm within the ML module 210 to determine a suitable predictive model for the input data.

The parameters module 212 iterates over a set of parameters for the predictive model generated by the model module 211 to generate a suitable value for each of the predictive model parameters. The predictive model parameters depend upon the type of the predictive model generated. For example, for an RF model, one of the predictive model parameters may be a number of decision trees, wherein each of the decision trees is a classification model, whereas for an SVM model, one of the predictive model parameters may be a type of a kernel, wherein the kernel is a set of mathematical functions for generalizing a non-linear classification problem. The parameter values may then be used to generate an ML algorithm for performing predictive analysis.

### (4) Reducing Computational Time by Way of Using Parallel Computing Network

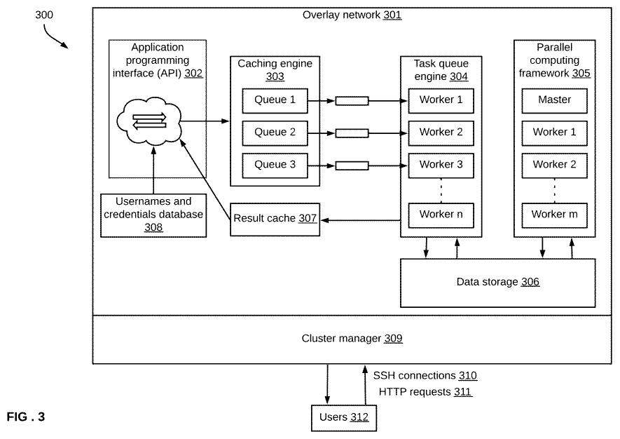

Referring now to FIG. 3, a block diagram of the ML framework 200 of FIGS. 2A-C functioning over a parallel computing network 300, implemented by the system 100 of FIG. 1, is illustrated, in accordance with some embodiments of the present disclosure. The parallel computing network 300 includes an overlay network 301 and a cluster manager 309.

The overlay network 301 includes an application programming interface (API) 302, a caching engine 303, a task queue engine 304, a parallel computing framework 305, and a data storage 306. The overlay network 301 is a framework for enabling parallel computing for a plurality of users 312.

The API 302 is a framework to allow the parallel computing network 300, access to the data source 201. As new data entries keep adding to the data source 201, the API 302 updates continuously after a particular time interval such that the parallel computing network 300 gets access to an updated data from the data source 201. The API 302 also allows the parallel computing network 300 access to a usernames and credentials database 308, wherein the usernames and credentials of a plurality of users, such as a plurality of employees or freelancers, may be stored. A results cache 307 is received by the API 302, wherein the results cache 307 is an access layer for a result obtained by one user allowing a faster access to the result for the other users.

The caching engine 303 is a data storage in a fast access memory hardware such as a Random Access Memory (RAM). When a data is retrieved from the data source 201 for the first time, a part of its information is stored as a cache in the caching engine 303. When the data is accessed for a successive time, the cache speeds up the data access for the users 312. The caching engine 303 may be based on Redis or any other data structure capable of running as a cache framework.

The task queue engine 304 is a data structure containing a list of tasks to be performed in the background. The tasks may be, retrieval of an updated data from the data source 201 or retrieval of results from the data storage 306. If the data from the data source 201 has been previously retrieved, the caching engine 303 allows a faster access to the data source 201 for the task queue engine 304. The task queue engine 504 may be based on Celery or any other task queue framework.

The parallel computing framework 305 is a framework to allow a plurality of users 312 to work together on a common input data. The parallel computing framework 305 also allows a containerized deployment of algorithms for a faster execution of the preprocessing, the feature selection, the predictive model, and an integration of multiple data types, wherein the integration of multiple data types is combining a plurality of datasets into a common dataset to obtain an increased set of features and a higher accuracy. The containerized deployment includes a plurality of containers or modules, each of which is deployed with at least one algorithm to execute. Each container may package an application together with libraries and other dependencies to provide isolated environments for running the application. The parallel computing framework 305 may be based on Apache Spark or any other parallel computing platform. The data and results obtained by the parallel computing framework 305 are stored in the data storage 306.

The data storage 306 is primarily accessible by the users 312. The data storage 306 is a relatively local data storage when compared to the data source 201. It may include the data received from the parallel computing framework 305 and the data received from the data source 201 via the task queue engine 304.

The cluster manager 309 receives a user query from at least one user 312 via a Secure Shell (SSH) connection 310 or a Hyper Text Transfer Protocol (HTTP) request 311 and sends the user query to the overlay network 301. The cluster manager 309 also receives an output from the overlay network 301 and sends the output to each of the users 312 via the SSH connection 310 or the HTTP request 311.

### (5) Iterative Preprocessing Input Data

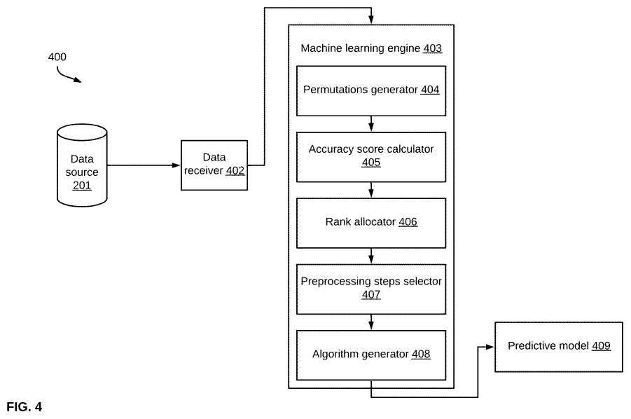

Referring now to FIG. 4, a block diagram of a preprocessing engine 400, implemented by the system 100 of FIG. 1, is illustrated, in accordance with some embodiments of the present disclosure. The preprocessing engine 400 includes a data source 201, a data receiver 402, an ML engine 403, and a predictive model 409.

The data source 201 is a system for storage of a data and provides an input data to the ML engine 403. Some examples include, but may not be limited to, a local storage data, a database, or a cloud storage data. The data receiver 402 receives the input data and identifies a data type of the input data. The input data is then transferred by the data receiver 402 to the ML engine 403.

The ML engine 403 further includes a preprocessing steps predictor 404, an accuracy score calculator 405, a rank allocator 406, a preprocessing steps selector 407, and an algorithm generator 408. The ML engine 403 contains a plurality of ML algorithms for different data types. The data receiver 402 identifies the data type of the input data and sends the information to the ML engine 403. One or more than one suitable ML algorithms can then be applied on various preprocessing parameters, based on the data type of the input data, to generate a specific and suitable preprocessing algorithm for the input data. The data types may include, but may not be limited to, Magnetic Resonance Imaging (MRI), functional Magnetic Resonance Imaging (fMRI) data, an Electroencephalogram (EEG) data, an Electrocardiogram (EKG/ECG) data, a genetics data, a proteomics data, data from wearable devices, an Electronic Health Record (EHR) data, and Electronic Medical Record (EMR) data, Chemistry (SMILES, InCHl, SDF), Images (PNG, JPEG) and other healthcare related data options. The preprocessing parameters may include, but may not be limited to, a pixel threshold, a linear/nonlinear regression, a volume threshold, and a smoothing method.

The preprocessing steps predictor 404 uses the ML algorithm to identify the data type and generate various permutations of the preprocessing parameters. These permutations are then applied on a test data (a subset of the input data) to check for their respective prediction accuracy scores by the accuracy score calculator 405. The accuracy score may be classification accuracy, logarithmic loss, confusion matrix, area under curve, F1 score, mean absolute error, mean squared error, or any other performance evaluation metric.

Classification accuracy is the ratio of number of correct predictions to the total number of predictions made. It can be represented as per equation (1) below:

Accuracy=Correct/Total, —(1)

where Correct=number of correct predictions made
Total=total number of predictions made
   Logarithmic loss penalizes false classifications and can be represented as per equation (2) below:

$$Log loss = \frac{- 1}{N} ( {\sum }_{i = 1}^{N} {\sum }_{j = 1}^{M} {y}_{ij} * log ( {p}_{ij} ) ) \qquad ( 2 )$$

where,
N samples belong to M classes
y_ij, indicates whether sample i belongs to class j or not
p_ij, indicates the probability of sample i belonging to class j
   Confusion matrix metric gives a matrix as an output describing the accuracy of each of the predictions made by the model. It sorts out each prediction as True Positives (TP), where the prediction as well as observation both were true, True Negatives (TN), where the prediction as well as observation both were false, False Positives (FP) where the prediction was true but the observation was false, False Negatives (FN), where the prediction was false but the observation was true. Accuracy for a confusion matrix can be represented as per equation (3):

Accuracy=(TP+TN)/(N) —(3)

Where, N=total number of samples

Area under curve (AUC) uses a curve called receiver operating characteristic (ROC) curve to evaluate the performance of a model. ROC curve is a plot of specificity vs sensitivity of a model where:

Specificity=(FP)/(FP+TN) —(4)

and Sensitivity=(TP)/(FN+TP) —(5)

Area under the ROC curve is calculated and a model with high AUC is considered better performing.

F1 score is a harmonic mean of precision and recall, where:

Precision=(TP)/(TP+FP) —(6)

Recall=(TP)/(TP+FN) —(7)

F1 score=2*(1/precision+1/recall)⁻¹ —(8)

Mean absolute error is the average of the difference between the observations and the predictions.

$$Mean absolute error = \frac{1}{N} {\sum }_{j = 1}^{N} \vert {y}_{j} - {\hat{y}}_{j} \vert \qquad ( 9 )$$

Where y_j is an observed value and y_j is a predicted value.

Mean squared error is the average of the square of the difference between the original values and the predicted values.

$$Mean squared error = \frac{1}{N} {\sum }_{j = 1}^{N} {( {y}_{j} - {\hat{y}}_{j} )}^{2} \qquad ( 10 )$$

The rank allocator 406 then arranges the various permutations in the decreasing order of their respective accuracy scores and assigns a rank in that order to each permutation or a predetermined number of permutations. The preprocessing steps selector 407 selects the top-ranked or a specified number of the permutations of preprocessing parameters. If more than one permutation is selected, the selected permutations may be displayed as options to the user. The user may then select a suitable option for a more customized preprocessing based on the research requirements. The algorithm generator 408 then uses the top-ranked or user selected permutation of preprocessing parameters to generate an optimized preprocessing algorithm. The predictive model 409 then performs data analysis using the optimized preprocessing algorithm.

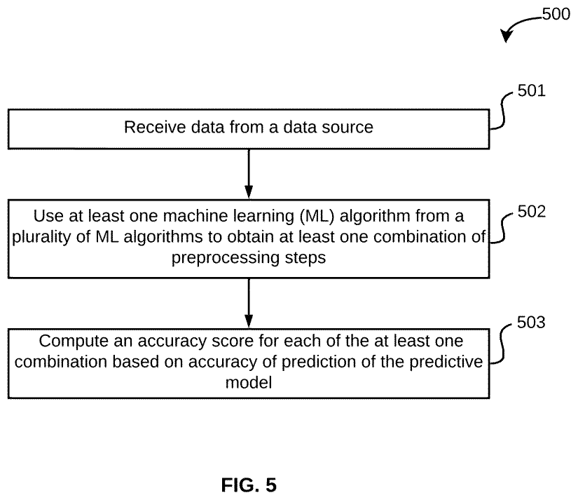

Referring now to FIG. 5, a flow diagram of an exemplary process 500 for preprocessing biomedical data, is illustrated, in accordance with some embodiments of the present disclosure. At step 501, the input data is received by the data receiver 402 from the data source 201. The data source 201 may be a part of the computer-readable medium 102 or one or more than one external device 105. The input data may be one or more than one large dataset. At step 502, at least one ML algorithm from a plurality of ML algorithms is applied, by the ML engine 403, on the preprocessing parameters to obtain at least one combination of preprocessing steps. The plurality of ML algorithms may include ML algorithms particularly created for biomedical data types, such as Magnetic Resonance Imaging (MRI), functional Magnetic Resonance Imaging (fMRI) data, an Electroencephalogram (EEG) data, an Electrocardiogram (EKG/ECG) data, a genetics data, a proteomics data, data from wearable devices, an Electronic Health Record (EHR) data, and Electronic Medical Record (EMR) data, Chemistry (SMILES, InCHl, SDF), Images (PNG, JPEG) and other healthcare related data options. At step 503, an accuracy score for each of the at least one combination of preprocessing steps is computed by the accuracy score calculator 405. The accuracy score may then be used as a basis for selecting a suitable combination of preprocessing parameters, leading to a suitable permutation of preprocessing steps.

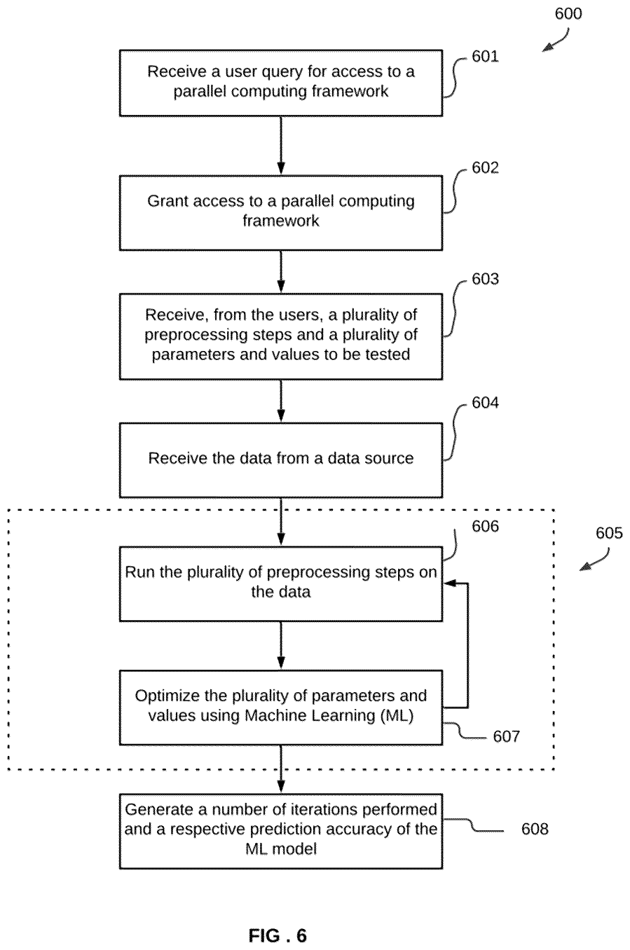

Referring now to FIG. 6, a flow diagram of an exemplary process 600 of preprocessing biomedical data using the parallel computing network 300 of FIG. 3, is illustrated, in accordance with some embodiments of the present disclosure. An ML process 605 is also depicted within the process 600. As illustrated in the flow diagram, at step 601 of the process 600, the parallel computing network 300 may receive a user query from the users 312 for access to the parallel computing framework 305. Consequently, at step 602, the parallel computing network 300 may then grant access to the parallel computing framework 305.

At step 603, the parallel computing framework 305 may receive, from the users 312, a plurality of preprocessing steps and the plurality of parameters and values to be tested for each of the preprocessing steps. The users 312 may define a sequence of the preprocessing steps. At step 604, once the sequence of the preprocessing steps is defined, the parallel computing framework 305 may receive the data from the data source 201 via the API 302.

The ML process 605 for preprocessing the input data is depicted in the flow diagram. Within the ML process 605, at step 606, the ML engine 403, implemented by the parallel computing framework 305, may run the plurality of preprocessing steps on the data. At step 607, the ML engine 403, implemented by the parallel computing framework 305, may optimize the plurality of parameters and values for each of the preprocessing steps of step 606 using an ML algorithm. The ML process 605 may be an iterative process wherein the plurality of parameters and values may be used in the preprocessing steps of step 606 and tested, on a test sample of the input data, for the associated prediction accuracy by using the accuracy score calculator 405.

At step 608, the parallel computing framework 305 may generate a number of iterations performed, using the plurality of parameters and values of each of the preprocessing steps, and a respective prediction accuracy of each of the iterations.

### (6) Data Integration and Feature Selection

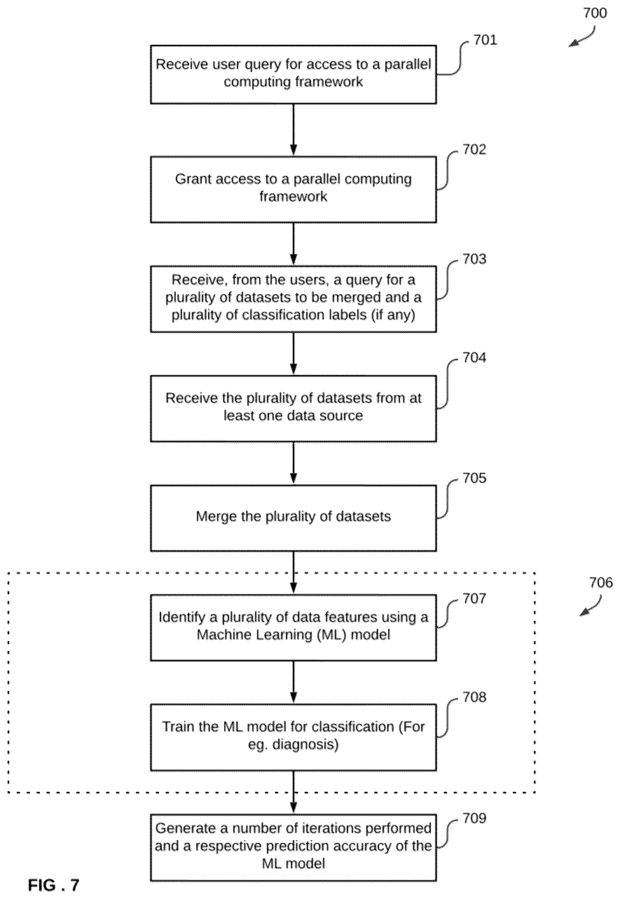

Referring now to FIG. 7, a flow diagram of an exemplary process 700 of merging a plurality of datasets and selecting relevant features from the combined dataset using the parallel computing network 300 of FIG. 3, is illustrated, in accordance with some embodiments of the present disclosure. A feature selection process 706 is also depicted within the process 700. As illustrated in the flow diagram, at step 701, the parallel computing network 300 may receive a user query from the users 312 for access to the parallel computing framework 305. Consequently, at step 702, the parallel computing network 300 may then grant access to the parallel computing framework 305.

At step 703, the parallel computing framework 305 may receive, from the users 312, a query for a plurality of datasets to be merged and a plurality of classification labels (if any). The plurality of datasets may have different data sources. At step 704, the parallel computing network 305 may receive the plurality of datasets from at least one data source. At step 705, the parallel computing network 305 may merge the plurality of datasets to give a combined dataset.

The feature selection process 706 for selecting the plurality of relevant features from the input data is depicted in the flow diagram. Within the feature selection process 706, at step 707, the parallel computing network 305 may identify a plurality of data features using a ML model. The ML model allows prediction of relevant data features, automating the feature selection process 706. At step 708, the parallel computing network 305 may train the ML model for classification problem such as diagnosis using the features obtained in step 707.

At step 709, the parallel computing network 305 may generate a number of iterations performed, using the features selected by the ML models of step 707, and a respective prediction accuracy of each of the ML models.

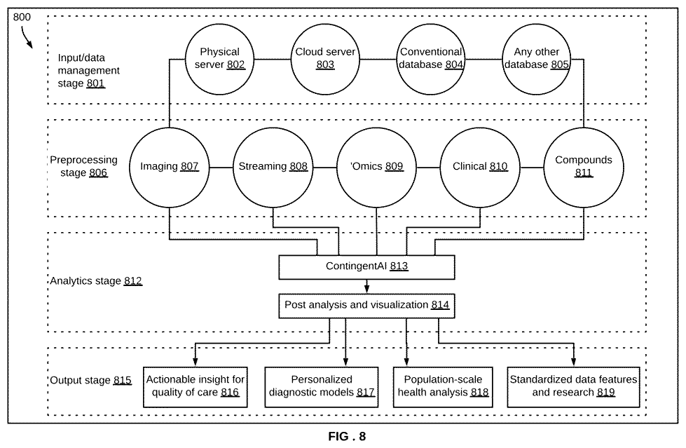

Referring now to FIG. 8, a block diagram of the examples of input sources and operations 800 performed by the parallel computing network 300 of FIG. 3, is illustrated, in accordance with some embodiments of the present disclosure. The examples of input sources and operations 800 of the parallel computing network 300 include the examples of an input/data management stage 801, a preprocessing stage 806, an analytics stage 812, and an output stage 815.

The examples of the input/data management stage 801 include a physical server 802, a cloud server 803, a conventional database 804, and an any other database 805. The examples of the preprocessing stage 806 include an imaging 807, a streaming 808, an omics 809, a clinical 810, and compounds 811.

The analytics stage 812 is implemented by a ContingentAI 813, wherein the ContingentAI 813 is an artificial intelligence (AI)/ML based framework for big data analytics of biomedical data. The post analysis and visualization 814 of the results are sent as output to the output stage 815.

The examples of the output stage 815 include an actionable insight for quality of care 816, personalized diagnostic models 817, a population-scale health analysis 818, and a standardized data features and research 819.

### (7) Variations on the Above Embodiments

It may be useful to arrange for the permutation generator 404 to generate ordered permutations based on previous rankings of configurations from the rank allocator 406.

It may be useful for the machine learning engine 403 to consider permutations in ranked order and to halt consideration when the accuracy score calculator 405 exceeds a specified threshold.

It may be useful to add pre-classified challenge data to the data source 201 in order to avoid certain sampling biases which may be present in the input data.

It may be useful to have the rank allocator 406 to weight accuracy scores 405 based on the accuracy of similar configurations against benchmarked data samples.

It may be useful for the machine learning algorithm 403 to evaluate the dependence or independence of choices in preprocessing 201 or feature selection 202. This evaluation may be used to reduce the total number of permutations to be examined.

It may be useful for the machine learning algorithm 403 to be seeded with rules or meta models for the selection of models 211 or hyperparameters 212 for the machine learning module 210.

It may be useful for the post analysis and visualization component 814 to present a plurality of results 213 as generated by different combinations of pre-processing steps and selections of features.

It may be useful for the post analysis and visualization component 814 to indicate areas of agreement or disagreement across models 210 generated by different combinations of pre-processing steps, feature selections, and model/hyperparameter settings.

It may be useful to arrange for the preprocessing engine 400 to accept pre-processing steps as defined by a particular programming language. The particular programming language can typically be a higher level programming language directed towards efficient coding of automated pre-processing tasks. It may be useful for the particular programming language to point out certain pre-processing tasks to be performed by the preprocessing engine.

### (8) Combining Preprocessing Steps and Integration of Heterogeneous Datasets

Biological samples by their nature are inherently variable and so when these are analysed it is generally necessary to apply data pre-processing procedures to compensate for these differences which may stem from sample size differences, unavoidable baseline shifts, missing data points, containing errors and outliers or lacking certain attributes or values.

There are many different algorithms available to choose from to establish a sensible pre-processing procedure. The procedure may include a data mining technique or algorithm that involves transforming raw data into a more processed format. They also may include methods to remove non-biological signal (bias) from the data that is introduced due to surveyor, equipment, or some other aspect of the experimental design. Below discussed are exemplary methods related to optimization of pre-processing data, and integration of these pre-processing methods with the machine learning process.

In some embodiments, the machine learning process comprises a preprocessing process, a training process, and a prediction process. The data processing process may extract training data from a database or a user, apply one or more transformations to standardize the training data, and pass the standardized training data to the training process. The training process may construct an assessment model based on the standardized training data. The prediction process may generate a predicted classification of the subject.

In some embodiments, the training process utilizes a machine learning algorithm to construct and train the assessment model.

In some embodiments, the prediction process generates the predicted classification of the subject by fitting new data to the assessment model, the new data being standardized by the preprocessing process. The prediction process may check whether the fitting of the new data generates a prediction of one or more specific disorders within a confidence interval exceeding a threshold value.

In some embodiments, the prediction process comprises a question recommendation process. The question recommendation process may identify, select, or recommend the most predictive next question to be asked with the subject, based on the plurality of answers to the plurality of asked questions, so as to reduce a length of assessment. The question recommendation process may select one or more candidate questions for recommendation as the next question to be presented to the subject. The question recommendation process may evaluate an expected feature importance of each one of the candidate questions. The question recommendation process may select one or more most predictive next question from the candidate questions, based on the expected feature importance of each one of the candidate questions. The expected feature importance of each one of the candidate questions may be determined with an expected feature importance determination algorithm.

The algorithm combines, feedback, activities, or interactions provided to select suitable combination of preprocessing steps, which can be ultimately customized by the user depending on need, origin of data, and other input. It may be used on human models or predictive algorithms.

The preprocessing module can be configured to apply one or more transformations to the extracted training data to clean and normalize the data, for example. The preprocessing module can be configured to discard features which contain spurious metadata or contain very few observations.

The preprocessing module can be further configured to standardize the encoding of feature values. Different datasets may often have the same feature value encoded in different ways, depending on the source of the dataset.

The preprocessing module can be configured to recognize the encoding variants for the same feature value, and standardize the datasets to have a uniform encoding for a given feature value. The preprocessing module can thus reduce irregularities in the input data for the training and prediction modules, thereby improving the robustness of the training and prediction modules.

In addition to standardizing data, the preprocessing module can also be configured to re-encode certain feature values into a different data representation. In some instances, the original data representation of the feature values in a dataset may not be ideal for the construction of an assessment model.

The preprocessing module can be further configured to impute any missing data values, such that downstream modules can correctly process the data. For example, if a training dataset provided to the training module comprises data missing an answer to one of the questions, the preprocessing module can provide the missing value, so that the dataset can be processed correctly by the training module. Similarly, if a new dataset provided to the prediction module is missing one or more feature values (e.g., the dataset being queried comprises only the answer to the first question in a series of questions to be asked), the preprocessing module can provide the missing values, so as to enable correct processing of the dataset by the prediction module.

The term ‘plurality of datasets’ can have several meanings. Datasets that have the same format, but are gathered using different machines of the same type can be considered different types of data.

Small differences in testing environments can also lead to different patterns in datasets which will be recognized by an ML algorithm as such, although undesired. Datasets that are gathered by different types of machines are also heterogeneous. The preprocessing module includes bias correction steps and parameters to homogenize data gathered by the same type of machine.

The preprocessing module could include different bias correction techniques to be varied, so that the type of bias correction could be included in the variation of preprocessing steps. Thus, the effect of each bias correction technique and each variation of parameters associated with the technique could be measured by calculating the accuracy of each predictive model quantity against the associated empirical quantities.

It is also important to integrate different types of data (i.e. data gathered by different types of machines) into the ML predictive model. It has been shown that using different types of data together in a single predictive model can lead to more accurate predictions. This can be achieved by allowing the preprocessing module to consider multiple heterogeneous datasets at the same time with the same algorithm.

This way, the preprocessing module can include variations of feature selection steps for each type of data. For example, a permutation of two types of feature selection on a single type of data would only lead to four types of permutations (neither, one, the other, or both). However, if two types of data are considered simultaneously and each type of data allows for two types of feature selection, the total number of permutation types would rise to sixteen.

The effects of each feature selection permutation on the combined dataset could also be measured by calculating the accuracy of each predictive model quantity against the associated empirical quantities. Thus, the machine learning preprocessing algorithm includes parameters for bias correction in order to homogenize datasets of the same format and feature selection parameters to simultaneously consider and integrate datasets of different formats for higher predictive accuracy.

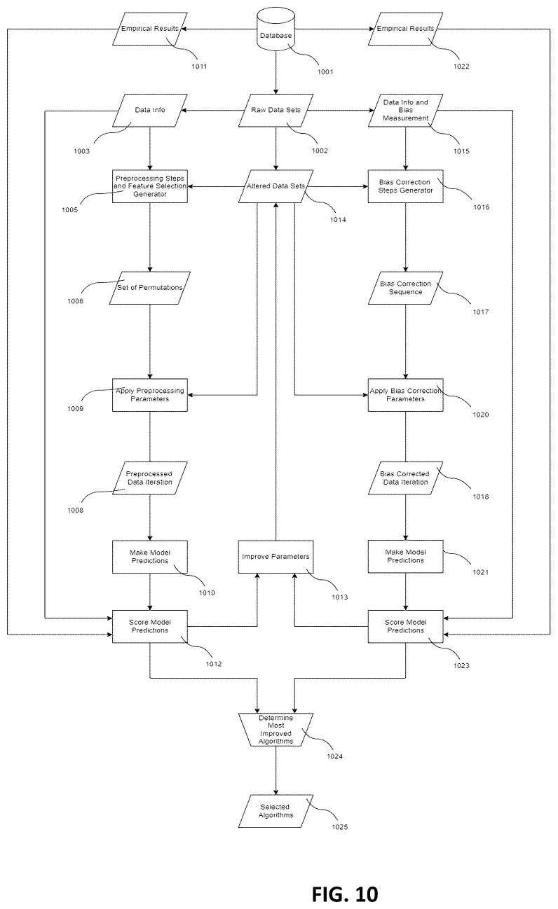

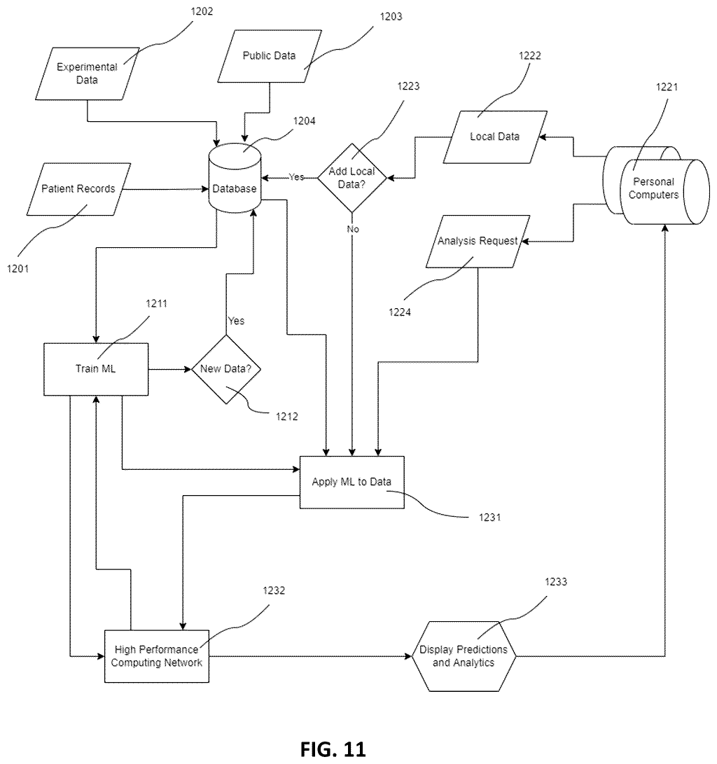

FIG. 10 is related to a flow diagram of an exemplary process of preprocessing parameter optimization included in the ML predictive model creation process.

Referring to FIG. 10, the database 1001 first gathers the raw data sets 1002 and sends administrative information about the data 1003 to the permutation generator 1005. The information about the data includes the type of data, the machine used to gather the data, the file format used to store the data, and any other relevant information that could be used by the permutation generator 1005.

The type of data could be anything relevant to the predictive model, but some examples are Magnetic Resonance Imaging (MRI), functional Magnetic Resonance Imaging (fMRI) data, an Electroencephalogram (EEG) data, an Electrocardiogram (EKG/ECG) data, a genetics data, a proteomics data, data from wearable devices, an Electronic Health Record (EHR) data, and Electronic Medical Record (EMR) data, Chemical Structures (SMILES, InCHl, SDF), Images (PNG, JPEG), including from pathology or other applications of microscopy, and other healthcare and medical research related data options.

The permutation generator 1005 then generates a number of preprocessing and feature selection steps, varying in both step selection and order of steps, and accompanying preprocessing parameters that are suitable for the type of data received. The set of generated permutations 1006 are sent to the algorithm generator 1007. The algorithm generator 1007 takes the permutation steps and turns each permutation into an algorithm that can be applied to the data. The full set of generated algorithms 1007 are sent to module 1009 to be applied to the data.

The raw data 1002 is sent to an editable placeholder module 1014, where the raw data can be altered without compromising the raw data, so that it can be reused after alteration if necessary.

The module 1009 takes the data from the placeholder module 1014 and applies the algorithms from module 1008. The algorithm application process results in a set of model predictions 1010 which can then be sent to module 1012. Module 1012 takes the set of model predictions 1010 as well as the administrative information about the data 1003 and finally the associated empirical results 1011. Module 1012 uses scoring techniques such as cross validation to compare the predicted quantities to the empirical quantities and calculates a number of accuracy measurements, including but not limited to classification accuracy, logarithmic loss, confusion matrix, area under curve, F1 score, mean absolute error, or mean squared error.

The accuracy scores are sent to module 1013, which aims to find the parameters that give the highest accuracy scores. The techniques used by this module include random search and grid search hyperparameter optimization. The process repeats by looping back to the data placeholder module 1014 and applying different preprocessing and feature selection algorithms, thereby generating different accuracy scores for each set of permutations.

At the same time as the preprocessing and feature selection permutation loop, other administrative information about the data 1015 and the placeholder data 1014 is sent to the bias correction steps generator 1016, where the focus is on debiasing the data.

Biases in datasets can include a selection bias, a reporting bias, a recall bias, an exclusion bias, an information bias, or a statistical bias. Most importantly, in biological data collection the most common bias is site bias, where even if the same experiment is conducted but across different locations, the resulting data will be skewed for various reasons.

Similarly, these non-biological signals can be attributed to confounding variations in personnel, date of data collection, well location etc. In order to mitigate these biases, the module 1015 can also use various algorithmic methods to quantify each type of relevant bias in each data set. For example, if the dataset came from a cohort study, the selection bias can be quantified by calculating the relative odds ratio and its standard error.

When collecting data for the study of various biological signals, it is possible for the signal to be corrupted in systematic ways due to the design of the experiments. In addition to the types of biases listed above, one common bias that the pre-processing steps aim to diminish can be referred to as site bias. If the same experiment is conducted across different locations, it is expected for the resulting data to be skewed in location A vs location B. Similarly, these non-biological signals can be attributed to confounding variations in personnel, date of data collection etc.

Bias is measured in several ways but strategies for accurate confounder analysis are not in abundance. Standard linear methods suffer from outlier sensitivity, are blind to complex structure in data, and cannot disambiguate hierarchically nested confounders; matching and stratification strategies suffer on high-dimensional data due to combinatorial scaling of unmatched dimensions, and matching is impossible for a lower-level confounder in a nested hierarchy; and Bayesian models suffer from poor performance in high dimensions.

Therefore, some embodiments may reduce non-biological signal and maximize biological signal concurrently through customized bias detection method as seen in module 1015 paired with iterative trials of varying batch correction algorithms as seen in module 1016 to 1020.

In low dimensional data, each variable can be plotted and visually attributed to each potential confounding variable.

In high dimensional data, even visual detection of bias is difficult and requires preprocessing the data using principal component analysis and subsequently visualizing whether data aggregate according to confounding variables in top-level components. This same strategy can be formed via any dimension reduction strategy (UMAP, t-SNE, etc.)

The bias correction steps generator takes the placeholder data 1014 and the data and bias info 1015 and generates a number of steps to correct the quantified biases to a suitable degree. For example, if the bias is a selection bias, a suitable bias correction method among a number of suitable methods could be an inverse probability weighting technique. If the data also possesses another quantified bias, like a statistical bias, a suitable bias correction method among a number of suitable methods could be a quantile mapping technique. For every quantified bias in a data set, the bias correction steps generator generates a number of permutations of bias correction steps, varying in both the bias correction technique and the order in which each step is applied, and accompanying bias correction parameters.

To give an example, if the model is more accurate at predicting the day of the week that sample is collected in contrast to a biological signal, it is considered for that dataset to be biased to towards non-biological signal.

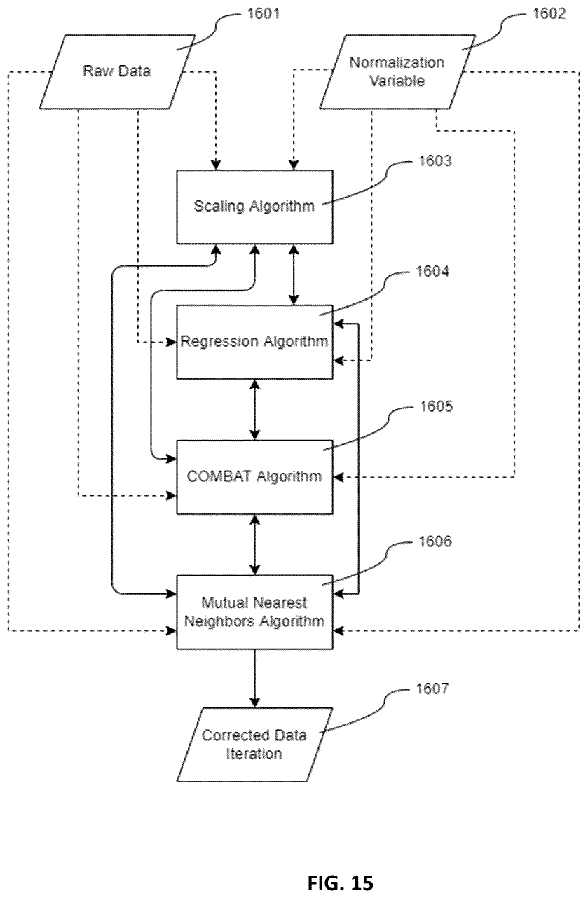

FIG. 15 is an exemplary iteration sequence of bias correction algorithms. The raw data 1601 and the normalization variable 1602, which can be any one of a full data set, plate ID, batch ID, date of experiment, or any other known batch effect, are sent to any of the modules 1603 to 1606.

If they are sent to 1603, a scaling algorithm is used to normalize the raw data 1601 according to the normalization variable 1602. The scaling algorithm 1603 can be a simple linear scaling algorithm or can be slightly modified to be robust to outliers. An example of a robust scaling algorithm is the RobustScaler from the Sklearn python module. If the raw data 1601 and normalization variable 1602 are sent to module 1604, a regression algorithm is used to normalize the raw data 1601 according to the normalization variable 1602. The regression algorithm 1604 can be a simple linear regression algorithm or can be slightly modified to prevent overcorrection. If the raw data 1601 and normalization variable 1602 are sent to module 1605, a COMBAT algorithm is used to normalize the raw data 1601 according to the normalization variable 1602. The COMBAT algorithm is a “supervised” batch effect algorithm that requires the bias to be quantified before applying a linear Bayes method to remove the bias. If the raw data 1601 and normalization variable 1602 are sent to module 1606, a mutual nearest neighbors algorithm is used to normalize the raw data 1601 according to the normalization variable 1602. The mutual nearest neighbors algorithm uses a nonlinear correction that quantifies systematic differences between groups of similar cells and scales the rest of the cells in the batches using this information.

The raw data 1601 can be sent to any of the modules 1603 to 1606, however, the normalized data from one module can be sent to another module for normalization. For example, the raw data can be sent to module 1603 where it is normalized by a scaling algorithm. In most references, corrected batch cases will be requested to proceed with mosty scaling factor to set the mean to 0 with unit variance. However, outliers can often influence the sample mean in a negative way. In such cases, approach implemented is slightly modified to be robust with outliers.

This normalized data can be sent to module 1604, where the normalized data is again normalized, but this time by a regression algorithm. Regress out is a commonly used technique to address batch effects, particularly in single cell transcriptomics datasets. This is a particularly effective sequence when there is a need to normalize the data, perform variance stabilization and regress out the effects of any covariates that have an effect on our data.

This twice normalized data can be sent to module 1605 where it is normalized by a COMBAT algorithm. This is a particularly effective sequence to remove batch effects in microarray data. Non-biological experimental variation or “batch effects” are commonly observed across multiple batches of microarray experiments, often rendering the task of combining data from these batches difficult. The ability to combine microarray data sets is advantageous to researchers to increase statistical power to detect biological phenomena from studies where logistical considerations restrict sample size or in studies that require the sequential hybridization of arrays. Finally, the normalized data can be sent to module 1606 and normalized by a mutual nearest neighbors algorithm. The result of such a normalization procedure is a bias corrected data iteration.

There can be any combination of data normalizations. For example, the normalization procedure can start with the raw data 1601, which is sent to module 1605, from which the normalized data can be sent to module 1603, from which the normalized data can be sent to module 1604, from which the normalized data can be finally sent to module 1602, from which another bias corrected data iteration can be obtained. The parameters associated with each bias correction algorithm can also be changed to produce a bias corrected data iteration. The results of the full set of iterated normalization procedures and iterated bias correction algorithm parameters is contained within the corrected data iterations 1607.

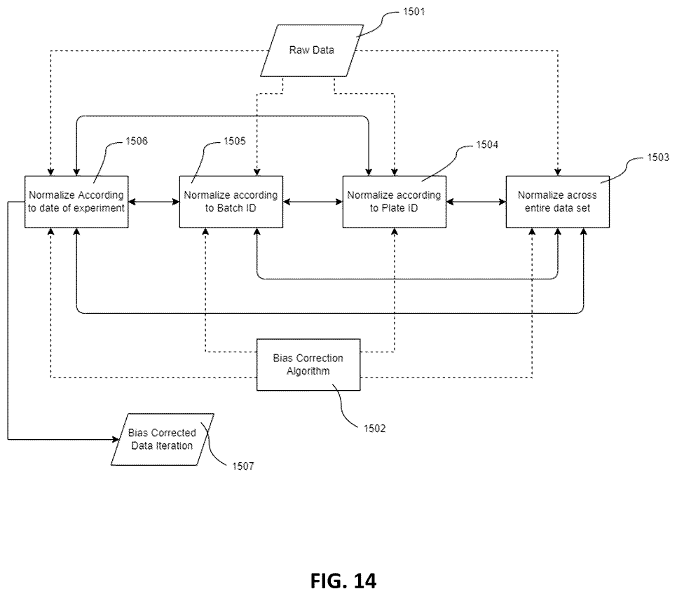

FIG. 14 is an exemplary bias correction iteration sequence for a single bias correction algorithm to normalize the data.

The raw data 1501 and the bias correction algorithm 1502, which could be any one of a scaling algorithm, regression algorithm, COMBAT algorithm, mutual nearest neighbors algorithm, or any other known batch effect correction algorithm, are sent to any of the modules 1503 to 1506.

If they are sent to 1503, the bias correction algorithm is used to normalize the data across the entire set. If they are sent to 1504, the bias correction algorithm is used to normalize the data according to the Plate ID, which is a type of batch effect, or nonbiological effect, originating from the small differences (sometimes microscopic differences) between cell culture plates. If they are sent to 1505, the bias correction algorithm is used to normalize the data according to the Batch ID, which is a type of batch effect originating from the small differences in cell batches. If they are sent to 1506, the bias correction algorithm is used to normalize the data according to the date that the experiment was conducted, which is another example of a batch effect.

The raw data 1501 can be sent to any of the modules 1503 to 1506, however, the normalized data from one module can be sent to another module for normalization. For example, the raw data can be sent to module 1503 where it is normalized across the entire data set. This normalized data can be sent to module 1504, where the normalized data is again normalized, but this time according to plate ID. This twice normalized data can be sent to module 1505 where it is normalized according to batch ID. Finally, the normalized data can be sent to module 1506 and normalized according to date of experiment. The result of such a normalization procedure is a bias corrected data iteration.

There can be any combination of data normalizations. For example, the normalization procedure can start with the raw data 1501, which is sent to module 1505, from which the normalized data can be sent to module 1503, from which the normalized data can be sent to module 1504, from which the normalized data can be finally sent to module 1502, from which another bias corrected data iteration can be obtained. The results of the full set of iterated normalization procedures is contained within the bias corrected data iterations 1507.

Now moving back to FIG. 10, there are several bias correction methods as explained above that are cocurring in module 1017. Once the bias is detected, there are several steps the algorithm may apply to correct bias that are caused by the non-biological factors are causing. In most references, corrected batch cases will be requested to proceed with mosty scaling factor to set the mean to 0 with unit variance. This approach implemented is slightly modified to be robust with outliers. Regress out is a commonly used technique to address batch effects, particularly in single cell transcriptomics datasets.

These bias correction steps 1017 are then sent to the algorithm generator 1018 to be converted to a set of algorithms 1019. As in the preprocessing and feature selection loop, the set of algorithms 1019 are applied to the placeholder data 1014 in module 1020, generating a number of model predictions. The set of model predictions 1021 can also include nonbiological predictions, like the day of the week the data was gathered, or the name of the doctor or scientist that gathered the data. The model predictions 1021 as well as the data and bias info 1015 and the associated empirical results 1022 are sent to the module 1021. When these model predictions 1021 are sent to the module 1023, accurate biological predictions score high, while accurate nonbiological predictions score low, as the goal of the debiasing loop is to remove the ability to make nonbiological predictions.

The accuracy scores are then sent to the module 1013 which aims to find the parameters that produce the maximum difference in predictive ability between biological predictions and nonbiological predictions. In other words, the module 1013 aims to find the parameters that simultaneously produce the most accurate biological predictions and the least accurate nonbiological predictions. As this module was described before, the techniques used to find such parameters include random search and grid search hyperparameter optimization. The process repeats by looping back to the data placeholder module 1014 and applying different bias correction algorithms, thereby generating different accuracy scores for each set of algorithms.

As explained above, there are two aspects to bias reduction: 1) using multiple approaches (scaling, regression, COMBAT, MNN) to attempt to remove non-biologcal signal, and then evaluating the effectiveness of these approaches using an ML framework. It is difficult to measure the effect of these de-biasing methods until the very end of the ML process. There, we examine the performance of a “true” model that looks to predict the outcome of interest, and a “bias” model, that looks to predict a non-biological feature of the data (e.g. survey date, surveyor, batch). What optimize for best performance of the “true” model, and worst performance of the “bias” model.

Once the module 1013 converges on the desired preprocessing, feature selection, and bias correction parameters, the information about the highest scoring parameters is sent to module 1024, where a user can either choose to select a set of preprocessing steps according to the accuracy scores and information about the application of the algorithms, such as speed or computational requirements, or to allow the algorithms to be automatically selected based on a chosen specialty, like highest speed algorithm, or most accurate algorithm. If more than one permutation is selected, the selected permutations may be displayed as options to the user. The outputs of this process are the selected algorithms 1025, which can then be applied to data to make predictions without the need for empirical validation.

The training module can utilize a machine learning algorithm or other algorithm to construct and train an assessment model to be used in the diagnostic tests, for example. An assessment model can be constructed to capture, based on the training data, the statistical relationship, if any, between a given feature value and a specific developmental disorder to be screened by the diagnostic tests. The assessment model may, for example, comprise the statistical correlations between a plurality of clinical characteristics and clinical diagnoses of one or more genetic, neurological, behavioral or mental health disorders.

A given feature value may have a different predictive utility for classifying each of the plurality of genetic, neurological, behavioral or mental health disorders to be evaluated in the diagnostic tests. The machine learning algorithm can be used to extract these statistical relationships from the training data and build an assessment model that can yield an accurate prediction of a develop mental disorder when a dataset comprising one or more feature values is fitted to the model.

In some instances, the diagnosis module can comprise data processing module as described herein. The data processing module can enable the diagnosis module to provide an assessment on the subject with reduced number of test questions. The data processing module can comprise a preprocessing module, a training module and a prediction module as described herein. The data processing module can extract training data from a database or a user, apply one or more transformations to standardize the training data and pass the standardized training data to the training module.

The training module can utilize a machine learning algorithm or other algorithm to construct and train an assessment model to be used in the diagnostic tests, based on the standardized training data. Once an assessment model is derived from the training data, the model may be used as a prediction tool to assess the risk of a subject for cognitive function such as developmental advancement, or one or more disorders such as behavioral, neurological or mental health disorders. The training data can comprise data developed on a population where the subject patient is not a member of the population. The prediction module can be configured to generate a predicted classification of cognitive function (e.g., developmental disorder) of a given subject, by fitting new data to an assessment model constructed in the training module. The data processing module can identify a most predictive next question based on a plurality of answers to a plurality of asked questions, as discussed herein, such that a person can be diagnosed or identified as at risk and treated with fewer questions.

One or more machine learning algorithms may be used to construct the assessment model, such as support vector machines that deploy stepwise backwards feature selection and/or graphical models, both of which can have advantages of inferring interactions between features.

For example, machine learning algorithms or other statistical algorithms may be used, such as alternating decision trees (ADTree), Decision Stumps, functional trees (FT), logistic model trees (LMT), logistic regression, Random Forests, linear classifiers, or any machine learning algorithm or statistical algorithm known in the art.

One or more algorithms may be used together to generate an ensemble method, wherein the ensemble method may be optimized using a machine learning ensemble meta-algorithm such as a boosting (e.g., AdaBoost, LPBoost, Tota!Boost, BrownBoost, MadaBoost, LogitBoost, etc.) to reduce bias and/or variance. Once an assessment model is derived from the training data, the model may be used as a prediction tool to assess the risk of a subject for having one or more behavioral, neurological or mental health disorders.

Machine learning analyses may be performed using one or more of many programming languages and platforms known in the art, such as R, Weka, Python, and/or Matlab, for example.

The training module may comprise feature selection. One or more feature selection algorithms (such as support vector machine, convolutional neural nets) may be used to select features able to differentiate between individuals with and without certain behavioral, neurological or mental health disorders. Different sets of features may be selected as relevant for the identification of different disorders. Stepwise backwards algorithms may be used along with other algorithms. The feature selection procedure may include a determination of an optimal number of features.

The training module may be configured to evaluate the performance of the derived assessment models. For example, the accuracy, sensitivity, and specificity of the model in classifying data can be evaluated. The evaluation can be used as a guideline in selecting suitable machine learning algorithms or parameters thereof. The training module can thus update and/or refine the derived assessment model to maximize the specificity (the true negative rate) over sensitivity (the true positive rate). Such optimization may be particularly helpful when class imbalance or sample bias exists in training data.

In at least some instances, available training data may be skewed towards individuals diagnosed with a specific disorder. In such instances, the training data may produce an assessment model reflecting that sample bias, such that the model assumes that subjects are at risk for the specific developmental disorder unless there is a strong case to be made otherwise. An assessment model incorporating such a particular sample bias can have less than ideal performance in generating predictions of new or unclassified data, since the new data may be drawn from a subject population which may not comprise a sample bias similar to that present in the training data.

To further reduce the contribution of training data sample bias to the generation of an assessment model, a boosting technique may be implemented during the training process. Boosting comprises an iterative process, wherein after one iteration of training, the weighting of each sample data point is updated. For example, samples that are misclassified after the iteration can be updated with higher significances. The training process may then be repeated with the updated weightings for the training data.

Some embodiments with a predictive modeling system include a predictive modeling exploration engine, a user interface, a library of predictive modeling techniques, and a predictive model deployment engine. The exploration engine may implement a search technique (or “modeling methodology”) for efficiently exploring the predictive modeling search space (e.g., potential combinations of pre-processing steps, modeling algorithms, and post-processing steps) to generate a predictive modeling solution suitable for a specified prediction problem.

The search technique may include an initial evaluation of which predictive modeling techniques are likely to provide suitable solutions for the prediction problem. In some embodiments, the search technique includes an incremental evaluation of the search space (e.g., using increasing fractions of a dataset), and a consistent comparison of the suitability of different modeling solutions for the prediction problem (e.g., using consistent metrics). In some embodiments, the search technique adapts based on results of prior searches, which can improve the effectiveness of the search technique over time.

The exploration engine may use the library of modeling techniques to evaluate potential modeling solutions in the search space. In some embodiments, the modeling technique library includes machine-executable templates encoding complete modeling techniques. A machine-executable template may include one or more predictive modeling algorithms. In some embodiments, the modeling algorithms included in a template may be related in some way. For example, the modeling algorithms may be variants of the same modeling algorithm or members of a family of modeling algorithms. In some embodiments, a machine-executable template further includes one or more pre-processing and/or post-processing steps suitable for use with the template's algorithm(s). The algorithm(s), preprocessing steps, and/or post-processing steps may be parameterized. A machine-executable template may be applied to a user dataset to generate potential predictive modeling solutions for the prediction problem represented by the dataset.

Library of predictive modeling techniques includes machine-executable templates encoding complete predictive modeling techniques. In some embodiments, a machine-executable template includes one or more predictive modeling algorithms, zero or more pre-processing steps suitable for use with the algorithm(s), and zero or more post-processing steps suitable for use with the algorithm(s). The algorithm(s), pre-processing steps, and/or post-processing steps may be parameterized. A machine-executable template may be applied to a dataset to generate potential predictive modeling solutions for the prediction problem represented by the dataset.

A template may encode, for machine execution, pre-processing steps, model-fitting steps, and/or post-processing steps suitable for use with the template's predictive modeling algorithm(s). Examples of pre-processing steps include, without limitation, imputing missing values, feature engineering (e.g., one-hot encoding, splines, text mining, etc.), feature selection (e.g., dropping uninformative features, dropping highly correlated features, replacing original features by top principal components, etc.). Examples of model-fitting steps include, without limitation, algorithm selection, parameter estimation, hyper-parameter tuning, scoring, diagnostics, etc. Examples of post-processing steps include, without limitation, calibration of predictions, censoring, blending, etc.

### (9) Use of a Cloud Network for Data Preprocessing

A cloud network allows for an application and data to be stored in a location other than the user's personal computing device. The application and associated data are instead stored in a computer system, in this case a parallel computing network, that is owned and operated by the cloud provider. A cloud network allows for a shared and organized database, which can be considered a proprietary piece or set of pieces of physical equipment.

The database can draw from public sources on the internet, private data streams from hospitals and healthcare providers, academic sources, and can even be expanded by a multitude of users that have access to local data. If the users can add local data to the shared database, the other users will be benefited by greater access to information and the machine learning algorithms will be made more accurate by the additional opportunity for refinement.

The machine learning algorithms can be constantly updated over a cloud network, so that the user does not need to use a personal machine to access a database and refine the software itself every time it needs to be updated. Since the cloud network allows the user access to remote computing resources, it can also connect the user to a remote parallel computing network that is owned and operated by the cloud service provider. Multiple users can connect to the parallel computing network at the same time. If multiple users that are connected to the parallel computing network at the same time desire the completion of common tasks, the network only needs to complete the task once, decreasing the computing resources required.

This is beneficial to both the cloud service provider, as fewer resources are needed, and also beneficial to users that request tasks that have been recently requested and completed by a different user. A cloud network also allows for the cloud service provider to gather data and statistics about the use of its software and the requirements of the users. This data can not only be used by the cloud service provider to allocate computing resources, but also to allocate business resources. Overall a cloud network makes the ML preprocessing algorithm refinement faster and more immediate, as well as granting easier and faster access to users.

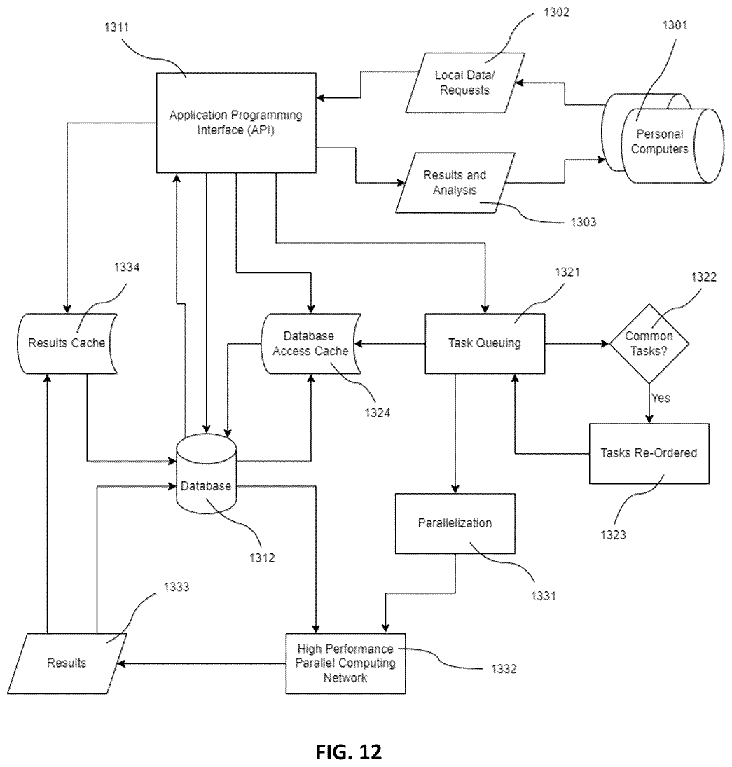

FIG. 12 is related to an exemplary process of utilizing cloud computing to enable data pre-processing, specifically in biomedical predictive model creation.

In FIG. 12, various types of biomedical data are gathered from healthcare providers and hospitals (1201), laboratories and academic journals (1202), and public internet databases (1203) and stored in a central database 1204 that is connected to the cloud network. The type of data could be anything relevant to the creation of a predictive model, but some examples are Magnetic Resonance Imaging (MRI), functional Magnetic Resonance Imaging (fMRI) data, an Electroencephalogram (EEG) data, an Electrocardiogram (EKG/ECG) data, a genetics data, a proteomics data, data from wearable devices, an Electronic Health Record (EHR) data, and Electronic Medical Record (EMR) data, Chemical Structures (SMILES, InCHl, SDF), Images (PNG, JPEG), including from pathology or other applications of microscopy, and other healthcare and medical research related data options.

Relevant data is used to train a machine learning engine 1211. The decision module 1212 represents a constant update loop, which checks if any new data is available in the database to be used to train the machine learning algorithm and updates the algorithm accordingly. The process of training the machine learning algorithm can be accomplished by a high performance computing network 1232 that is owned and operated by the cloud service provider. A group of personal computers 1221, which are simultaneously connected to the cloud network, can send local data 1222 or set of tasks 1224 to the cloud network. The set of tasks contains all of the necessary information, apart from biomedical data, for the analysis to take place, including which data to gather from the database, the type of predictive model required, and the type of analysis to run on the quantitative prediction results.

The user can also specify in the tasks module whether they would like to use an existing trained machine learning algorithm or to train a new machine learning algorithm from a specific dataset. The local data 1222 can be added to the biomedical database for access by other users, or be sent straight to the machine learning algorithm 1231 so that the data remains private, represented by a decision module 1223. The machine learning engine then gathers the local data or shared data from the database as well as the requests from the users, and applies the chosen machine learning algorithm 1211 to the data. The process of applying the machine learning algorithm to the data and the analysis of the results can be accomplished by a high performance computing network 1232 that is owned and operated by the cloud service provider. The predictive results and analyses 1233 are sent back to the personal computers so that they can be visually displayed to the users.

All data will be automatically sent to the cloud storage system. All data sent and stored in the cloud platform will be stored anonymously. No personal identifying information will be stored and patients will be assigned a numeric-alpha patient identifier code. In addition the cloud platform will include a set of visualization tools such as interactive charts and plots of the evolution of the monitored data over time to provide a greater amount of information.

All data will automatically be stored in a secure cloud storage platform. As soon as patient and user profiles are created, all personal information will be anonymized and only accessible to appropriate parties in password protected archive records.

The storage system is coupled to the cloud services provider via a data communications link. The data communications link may be embodied as a dedicated data communications link, as a data communications pathway that is provided through the use of one or data communications networks such as a wide area network (‘WAN’) or local area network (‘LAN’), or as some other mechanism capable of transporting digital information between the storage system and the cloud services provider. Such a data communications link may be fully wired, fully wireless, or some aggregation of wired and wireless data communications pathways.

In such an example, digital information may be exchanged between the storage system and the cloud services provider via the data communications link using one or more data communications protocols. For example, digital information may be exchanged between the storage system and the cloud services provider via the data communications link using the handheld device transfer protocol (‘HDTP’), hypertext transfer protocol (‘HTTP’), internet protocol (‘IP’), real-time transfer protocol (‘RTP’), transmission control protocol (‘TCP’), user datagram protocol (‘UDP’), wireless application protocol (‘WAP’), or other protocol.

The cloud services provider may be embodied, for example, as a system and computing environment that provides services to users of the cloud services provider through the sharing of computing resources via the data communications link. The cloud services provider may provide on-demand access to a shared pool of configurable computing resources such as computer networks, servers, storage, applications and services, and so on. The shared pool of configurable resources may be rapidly provisioned and released to a user of the cloud services provider with minimal management effort.

Generally, the user of the cloud services provider is unaware of the exact computing resources utilized by the cloud services provider to provide the services. Although in many cases such a cloud services provider may be accessible via the Internet, readers of skill in the art will recognize that any system that abstracts the use of shared resources to provide services to a user through any data communications link may be considered a cloud services provider.

The cloud services provider may be configured to provide a variety of services to the storage system and users of the storage system through the implementation of various service models. For example, the cloud services provider may be configured to provide services to the storage system and users of the storage system through the implementation of an infrastructure as a service (‘IaaS’) service model where the cloud services provider offers computing infrastructure such as virtual machines and other resources as a service to subscribers. In addition, the cloud services provider may be configured to provide services to the storage system and users of the storage system through the implementation of a platform as a service (‘PaaS’) service model where the cloud services provider offers a development environment to application developers.

Such a development environment may include, for example, an operating system, programming-language execution environment, database, web server, or other components that may be utilized by application developers to develop and run software solutions on a cloud platform.

Furthermore, the cloud services provider may be configured to provide services to the storage system and users of the storage system through the implementation of a software as a service (‘SaaS’) service model where the cloud services provider offers application software, databases, as well as the platforms that are used to run the applications to the storage system and users of the storage system, providing the storage system and users of the storage system with on-demand software and eliminating the need to install and run the application on local computers, which may simplify maintenance and support of the application.

The cloud services provider may be further configured to provide services to the storage system and users of the storage system through the implementation of an authentication as a service (‘AaaS’) service model where the cloud services provider offers authentication services that can be used to secure access to applications, data sources, or other resources.

The cloud services provider may also be configured to provide services to the storage system and users of the storage system through the implementation of a storage as a service model where the cloud services provider offers access to its storage infrastructure for use by the storage system and users of the storage system.

Cloud migration tools may also be configured to address potentially high network costs and long transfer times associated with migrating large volumes of data to the cloud services provider, as well as addressing security concerns associated with sensitive data to the cloud services provider over data communications networks. In order to further enable the storage system and users of the storage system to make use of the services provided by that may be offered by the cloud services provider or a limitation as to the service models that may be implemented by the cloud services provider.

### (10) Use of Parallel Computing Cloud Network for Data Preprocessing

Using a parallel computing network to create and run a predictive model is technically more difficult, but superior in many areas to using a singular instance of a program on one machine to accomplish the same goal. An API is used to connect a user to a parallel computing network, and to connect a parallel computing network to a database. The parallel computing network receives tasks from the user and executes the tasks while accessing the designated data from the database, sending the results back to the user.

The tasks can include variation of preprocessing steps and parameters, execution of preprocessing steps, feature selection, and machine learning model creation and training. The algorithms used to execute the tasks can be parallelized to allow for faster completion of tasks and more balanced usage of computing resources. For example, if the required task is matrix multiplication, instead of using a traditional sequential algorithm, the parallel computing network can use a divide-and-conquer algorithm such as a Strassen algorithm on multiple processors to significantly speed up the completion of the task.

Parallelizing sequential algorithms is not a simple task and its difficulty or possibility is completely dependent on the algorithm. Aside from algorithmic parallelization, machine learning model training can also be parallelized.

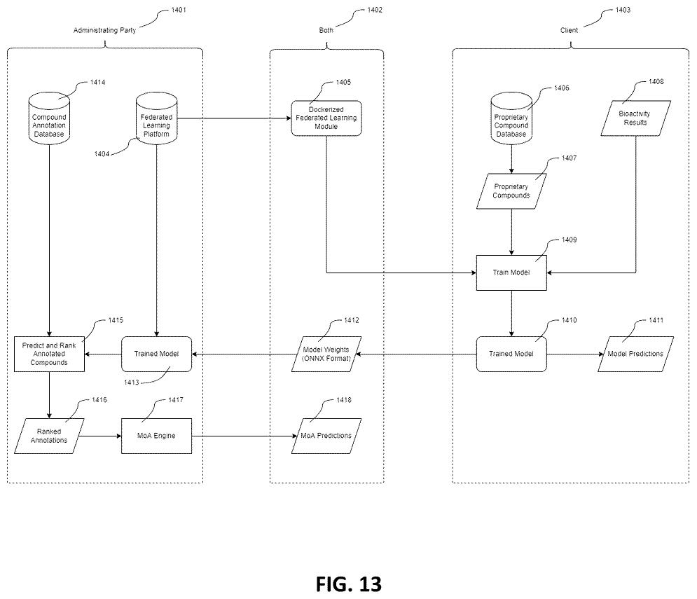

FIG. 13 is related to an exemplary process for using parallel computing networks and associated API specifically for data preprocessing, specifically for biomedical predictive model creation over a cloud network.

Now referring to FIG. 13, the personal computers 1301 send local data and requests 1302 to the API 1311. The local data is sent to a remote database 1312 and the caching engine 1324 stores information about how to access the data quickly. The requests are sent to the task queuing engine 1321. The tasks can include variation of preprocessing steps and parameters, execution of preprocessing steps, feature selection, or machine learning algorithm, machine learning model creation and training, and retrieving data or results from a database. The decision module 1322 searches the task queue for common or similar tasks and 1323 reorders the queue so that the same task does not need to be completed repeatedly.

The first task in the queue is sent to the parallelization module 1331, which decides how to parallelize the task. If the task were a simple data transformation, like a matrix multiplication process, a suitable parallel method would be selected to complete the task in a parallel computing framework. For the example of matrix multiplication, instead of using a traditional sequential algorithm, the parallel computing network can use a divide-and-conquer algorithm such as a Strassen algorithm on multiple processors to significantly speed up the completion of the task.

If the task were the execution of a machine learning training process, the parallelization module could select one of two methods, data parallelism or model parallelism. If the parallelization module selects data parallelism, the same machine learning model is trained on different processors by dividing the training data and sending each data division to a separate instance of the same machine learning algorithm. If the parallelization module selects model parallelism, the machine learning model itself is divided into different layers to be trained on the same data set. If the first task in the queue involves retrieving data from the database, it is sent to the caching engine 1324 to speed the retrieval. The data from the database, along with the parallelization instructions, are sent to the high performance parallel computing network 1332.

The results of the computations 1333 are sent to the database for storage, and the storage information is sent to the results cache 1334, so that users looking for results in the database can be found easily. The results can include prediction quantities, accuracy scores, preprocessing algorithms, preprocessing parameter values, and computational information like required resources or computation time.

If a user request from module 1302 received by the API 1311 is the retrieval of results, the result cache 1334 assists the API to find them in the database 1312. The results are then sent from the database to the API. If the analysis of the data requires more high performance computing, the necessary analysis algorithms are sent to the task queue to be sent to the parallel computing network.

The API then retrieves the results and analysis 1303 and sends them to the desired personal computers on the cloud network. If the data analysis only requires minimal computing power, the API sends the raw data to the personal computers so that they can analyze the data independently. If the user only desires to access and analyze data from the database, the API can directly access the database cache and retrieve raw data from the database to send to the user.

Data transmission network may also include one or more cloud networks. Cloud network may include a cloud infrastructure system that provides cloud services. In certain embodiments, services provided by the cloud network may include a host of services that are made available to users of the cloud infrastructure system on-demand. Cloud network is shown as being connected to computing environment (and therefore having computing environment as its client or user), but cloud network may be connected to or utilized by any of the devices.

Services provided by the cloud network can dynamically scale to meet the needs of its users. The cloud network may comprise one or more computers, servers, and/or systems.

In some embodiments, the computers, servers, and/or systems that make up the cloud network are different from the user's own on-premises computers, servers, and/or systems. For example, the cloud network may host an application, and a user may, via a communication network such as the Internet, on-demand, order and use the application.

Using parallel computing network and allowing transportation of a model between users of the platform carries great significance. However, proecting molecular structures from disclosure against external parties also carries prominence in this framework.

In some embodiments, the process may require the algorithmic model to directly ingest and process chemical structure information, which can be sensitive and proprietary data.

Securely exchanging chemical data without revealing the molecular structure is of great importance, as sharing data such as fingerprints and measured endpoints between research groups within industry through collaboration is often accomplished to improve drug discovery.

It is common in pharmaceutical and biomolecular industries to exchange datasets by encoding the molecular structures into descriptors. Molecular fingerprints such as the extended-connectivity fingerprints (ECFPs) are frequently used for such an exchange, because they typically perform well on quantitative structure-activity relationship tasks.

The ECFP representation is a refinement of the Morgan algorithm and usually hashed and folded into a fixed size 1024, 2048 or 4096 sparse bit or count vector to further utilize for predictive modeling tasks. During the fingerprint creation, the ECFP algorithm considers the atom environment, based on the maximum number of atomic neighbors, i.e. bond diameter d, and iteratively hashes the concatenated (unique) features to a new integer feature. Since the hash function is mapping randomly and uniformly to a 232-size space of integers, the ECFPs are often considered to be non-invertible

However, there has been reverse-engineering methods in prior art that may deduce the molecular structure given revealed ECFPs which would jeopardize the sanctity of intellectual property of private companies and structure disclosure that would restrict individual scientists in a federated learning module.

Therefore some embodiments may feature a customized federated learning module that allows model training to be conducted securely on client machines without external access while still allowing the administrator to use the trained model.

FIG. 13 is related to information exchange process for federated learning models that ensures bi-directional data security.

The dashed box labeled 1401 shows all of the data the administrator has access to, the dashed box labeled 1403 shows all the data the client has access to, and the dashed box labeled 1402 shows all the data both parties have access to.

Starting at box 1404, a database stores the federated learning platform, some of which may be proprietary to the administrating party. From this database, a containerized federated learning module via Docker in 1405 can be extracted and shared among both parties.

Docker is a containerization platform which is used to package an application and all its dependencies together in the form of containers so to make sure that the application works seamlessly in any environment which can be in development or test or production.

In essence, containers share the same host kernel but are isolated from each other through private namespaces and resource control mechanisms at the OS level. Container-based virtualization provides a different level of abstraction in terms of virtualization and isolation when compared with hypervisors. Hypervisors use a lot of hardware which results in overhead in terms of virtualizing hardware and virtual device drivers.

Containers implement isolation of processes at the operating system level, thus avoiding such overhead. These containers run on top of the same shared operating system kernel of the underlying host machine and one or more processes can be run within each container.

There are several other advantages to utilizing Docker in addition to its promise to solve the privacy-preserving federated machine learning.

One advantage Docker offers is speed. The speed of Docker containers compared to a virtual machine is very fast. The time required to build a container is very fast because they are tiny and lightweight. Development, testing, and deployment can be done faster as containers are small. Containers can be pushed for testing once they have been built and then from there on to the production environment.

Another advantage Docker offers is portability. The applications that are built inside docker containers are extremely portable. These portable applications can easily be moved anywhere as a single element and their performance also remains the same.

Another advantage Dockers offers is density. Docker uses the resources that are available more efficiently because it does not use a hypervisor. This is the reason that more containers can be run on a single host as compared to virtual machines. Docker Containers have higher performance because of their high density and no overhead wastage of resources.

The client will have a secure database 1406 that stores information about proprietary compounds. The proprietary compounds data 1407 can be extracted from the database and, along with the federated learning module 1405 and empirical bioactivity results 1408, can be sent to module 1409 where a chemical structure-activity model is trained. The resulting trained model 1410 can be used to generate predictions 1411 that can stay internal to the client.

In order to keep the information about the proprietary compounds secure, the trained model is kept as an internal entity to the client, while the model weights in Open Neural Network Exchange (ONNX) format 1412 are shared with the administrating party.

ONNX is a set of tools used to better hangle storing, transferring and deploying model architectures and trained weights. Specifically, ONNX was designed to be cross platform, allowing models to be trained and deployed in different contexts. Although it was originally developed for neural networks, this set of tools has been expanded to cover tree-based models as well as which are used extensively in this process.

The administrating party can then use the model weights 1412 as well as the federated learning platform 1404 to create their own trained model 1413. In this way, the administrating party can gain insight from the client's model without compromising the security of the client's proprietary data.

The administrator can then use their trained model 1413 and their own compound annotation database 1414 to predict the phenotypic characteristics of compounds and rank the predictions 1415. The ranked annotations 1416 can then be processed through the mechanism of action engine 1417 to generate mechanism of action predictions 1418 that can be used by either party without compromising the security of any proprietary data.

Parallel computing may also enable that multiple devices be used simultaneously. For example, a set of network devices can be used to transmit various communications from a single user, or remote server may include a server stack. As another example, data may be processed as part of computing environment. Each communication within data transmission network may occur over one or more networks. Networks may include one or more of a variety of different types of networks, including a wireless network, a wired network, or a combination of a wired and wireless network. Examples of suitable networks include the Internet, a personal area network, a local area network (LAN), a wide area network (WAN), or a wireless local area network (WLAN).

Some aspects may utilize the Internet of Things (IoT), where things (e.g., machines, devices, phones, sensors) can be connected to networks and the data from these things can be collected and processed within the things and/or external to the things. For example, the IoT can include sensors in many different devices, and high value analytics can be applied to identify hidden relationships and drive increased efficiencies. This can apply to both big data analytics and real-time (e.g., ESP) analytics.

In data parallelism, the same machine learning model is trained on different processors by dividing the training data and sending each data division to a separate instance of the same machine learning algorithm. In model parallelism, different layers of one model can be trained on different processors, which is similar to the above example of parallelization of a mathematical sequential algorithm and is more complex than data parallelism. In this method, the ML algorithm itself is divided into smaller pieces by algorithmic parallelization and exposed to the same dataset. Some of the data retrieved from the database and even some of the results sent back to the user can also be stored in a caching engine.

This allows for future users to access common data for the completion of the same task and even to aid completion of similar tasks. Tasks that the user requests the parallel computing network to complete are sent to the task queue. The task queue can identify where there is any overlap for multiple users. If there is overlap, the tasks can be further divided so that each instance of overlap only needs to be completed once.

### (11) Software for Compiling and Determining Relationships Between Biological Data

Some embodiments of the present disclosure pertain to a method for compiling and determining relationships between biological data. In particular, the use of prioritized genomic data to determine genetic candidates suitable for phenotypic testing.

The last decade has seen a rapid increase in the adoption of genome sequencing in the clinical diagnosis of genetic diseases. To efficiently detect pathogenic variants and to improve the diagnostic rate of the genome-based approach, it is necessary to have disease-gene prioritization that substantially reduces the number of candidate causal variants and ranks them for further interrogations based on the association of the corresponding genes with the disease phenotype. In other words, the disease-gene prioritization is the process of assigning a likelihood of gene involvement in generating a disease phenotype.

A major area in the field of genomic medicine aims to identify harm caused by genetic variants in an organism using phenotypic data. Gene Disease Prioritization (GDP) serves to identify and prioritize various genes pertaining to certain genetic diseases, conditions, and disorders to be used in experimental testing. Some embodiments pertain to methods of using artificial intelligence (AI), machine learning (ML), and other algorithms to produce genetic candidates that may cause certain diseases or conditions. These methods may help narrow down potential disease-causing genes from an untestable amount to a more manageable amount for phenotypic testing.

Embodiments of the present disclosure generally relate to a phenograph. As used herein, a phenograph is a platform or database containing a computationally efficient algorithm or clustering method for use in compiling biological data. This biological data may include but is not limited to phenotypes, genes, gene ontology, genome sequencing, orthology, protein data, high dimensional single-cell data, biological relationships, or other biological data.

The phenograph in the preferred embodiment helps users to sort through biological data to determine genetic candidates that relate to genetic diseases to identify the candidates most likely to show abnormal phenotypes such as enlarged heart, blindness, small lungs, low blood flow, underdeveloped organs, or all other current and future phenotypes.

As used herein, a phenograph is a platform or database containing a computationally efficient algorithm or clustering method for use in compiling biological data. This biological data may include but is not limited to phenotypes, genes, gene ontology, genome sequencing, orthology, protein data, high dimensional single-cell data, biological relationships, or other biological data.

In some embodiments, the biological data is compiled through extraction from biological databases. These databases may include phenotypes, genes, gene ontology, genome sequencing, orthology, protein data, high dimensional single-cell data, biological relationships, or other biological data. The data may also include gene, phenotype, and disease relationship data between animal species that share similar genes with humans, such as mice, rats, other rodents, other mammals, or other animals. The present disclosure puts an emphasis on zebrafish because humans and zebrafish share 70 percent of the same genes and 84 percent of human genes known to be associated with human disease have a counterpart in zebrafish. Zebrafish also share major organs and tissues with humans making it easier to predict genes that may cause harmful phenotypes in humans using zebrafish orthologs. Other embodiments may pertain to a phenograph that focuses on gene, phenotype, and disease relationships between humans and another species such as the aforementioned examples.

The phenograph connects to biological databases and uses the data extracted from those databases to determine relationships between genetic variants, phenotypes, and diseases. The phenograph is a valuable tool because it can be used to predict how a change in a gene may affect a phenotype or disease. By understanding these relationships, researchers can better understand the causes of diseases and develop new treatments. For example, if a researcher wants to know how a mutation in the gene BRCA1 may affect breast cancer risk, they can use the phenograph to look at the relationship between the BRCA1 gene and breast cancer.

### (12) Gene Disease Prioritization

Gene prioritization is the process of assigning likelihood on gene involvement in generating a disease phenotype.

The process of gene-disease prioritization narrows the possible genes and gene variants likely to cause certain diseases. This approach may also arrange the genes and gene variants into an order of likelihood in disease involvement. After prioritization, the amount of possible candidate genes is now more manageable for phenotype and genetic testing. Gene orthology can be used to determine the best candidates for testing. This testing is usually performed on animals with genes that have human orthologs.

Some embodiments may include assigning similarity or confidence scores to genes and ranking them based on the probability of their association with the disease of interest. This allows for a more accurate determination of which genes are most likely to be associated with the disease, and formats them in an way easily read and understood by humans and machines.

Some embodiments use AI and ML models to determine the genes likely involved in certain diseases. Disease genes are found by these models because they tend to interact with other disease genes that are often involved in the same biological pathways, networks, or sections of DNA. Disease genes also tend to harbor detrimental mutations. These mutations can disrupt the normal function of the gene, leading to diseases. Disease genes also tend to code for proteins that localize to the affected biological compartment (pathway, cellular space, or tissue). Disease genes tend to have distinct sequence properties, such as longer length and a higher number of exons. This is because these properties can make the gene more difficult to mutate and more likely to be involved in disease. Disease genes tend to have more orthologues and fewer paralogues. Orthologues are genes that share a common ancestor, while paralogues are genes that share a common ancestor but have since diverged.

In a preferred embodiment, there is a Gene Disease Prioritization (GDP) model that predicts certain disease-related genes that are likely to cause a relevant phenotype in zebrafish (Danio rerio). This platform extracts data gathered from Ensembl, ZFIN.org, and RNA-sequence time series gene expression from White Et. al. It should be understood that information from other databases could be extracted and used. The information may also be input manually instead of automatically.

In a preferred embodiment, genes, phenotypes, and other biological data is taken from zebrafish (Danio Rerio) because humans and zebrafish share 70 percent of the same genes and 84 percent of human genes known to be associated with human disease have a counterpart in zebrafish. Zebrafish also share major organs and tissues with humans making it easier to predict genes that may cause harmful phenotypes in humans using zebrafish orthologs.

In a preferred embodiment, the software is supported by a comprehensive linkage map that empowers translation of disease biology by connecting genes to phenotypes to model systems to drugs. Users can query any stage of drug discovery, starting with clinical insights or phenotypic hits, and connect data or fill gaps, to improve translation predictions. One of the major data sources is Ensembl, a database for genome sequencing, protein domains, orthology of multiple organisms, Gene Ontology (GO), and Kyoto Encyclopedia of Genes and Genomes (KEGG). The data and information pertaining to GO, KEGG, Orthology, Protein Family Domains (PFAM), and Protein Sequences are compiled to generate a diverse feature matrix with rows to describe each gene at the “Concatenate” step. ProteinBERT, a trained protein language model, accepts inputs of Protein Sequences and outputs Protein Sequence Embeddings/Representations as nodes, each node pertaining to a single protein sequence. This data allows for graphing models and neural network models to be created from the protein sequences. The Protein Sequence Embeddings are concatenated with the other gene data extracted from Ensemble. FIG. 16 shows GO data 2130, KEGG data 2131, Orthology data 2132, and Protein Domains 2133 being extracted from Ensembl 2111 and concatenated at the Concatenate step 2150.

In some embodiments, protein sequence embeddings are generated via a protein language model or deep-learning model, such as ProteinBERT, UDSMProt, Unirep, or other current and future protein language models. In a preferred embodiment, ProteinBERT is previously trained on all available protein sequences to ingest protein sequence information and predict protein function. This is trained on all available protein sequences annotated with gene ontology labels. The protein function predictions are not needed, instead, the inner representation for how ProteinBERT interprets protein sequences is used. ProteinBERT takes a protein sequence of amino acid letters and creates a numerical internal representation of the protein sequence. The internal representation is then converted into an output protein function prediction used to assign a biological role to the protein. The process may also determine the biochemical role of the protein when in contact with certain chemicals, compounds, drugs, etc.

In a preferred embodiment, ProteinBERT is run on all zebrafish genes (compounded and extracted from external databases such as Ensembl and ZFIN.org) to capture the internal representation. This way, another useful descriptor of each zebrafish gene is created. This internal representation doesn't exactly represent any particular trait of the protein (e.g. structure, function, etc.), but instead serves as a proxy to capture key information relating to all of that information.

Since the ProteinBERT internal structure already contains all the relevant protein information in a machine-friendly format, there is no need to try and find this information manually, which ends up saving time. Specifically, each protein sequence of interest is inputted into ProteinBERT, and the internal representation (a.k.a. hidden layers) are extracted into a linear feature matrix. FIG. 16 shows Protein sequences data 2136 being extracted from Ensembl 2111 and being sent to the feature matrix in the Concatenate step 2150. The Protein sequences data 2136 is also sent to ProteinBERT 2140 to be converted into Sequence embeddings 2141 and concatenated with the other Ensembl 2111 data in the Concatenate step 2150.

In some embodiments of the Concatenation step, the features extracted from certain databases features are gathered for each gene. Features include descriptors such as expression levels at 24 hours post fertilization, GO terms, etc. These are typically either numerical (RNA time series expression), categorical variables (GO, KEGG, Protein Domains) or binary variables (Orthology, ZFIN expression). Certain types of information is preprocessed before being entered into the feature matrix. Categorical information is hot encoded to generate a series of binary labels per category. Information held in strings (such as protein sequence information) is translated by ProteinBERT to generate a series of numerical variables which are then directly concatenated into the feature matrix. Preprocessing is not needed for Numerical and binary information such as RNA time series expression, Orthology, and ZFIN expression.

Another major data source is ZFIN.org, a database for zebrafish genes, gene expressions, mutations, antibodies, labeled anatomy, nucleotides, protein sequences, etc. ZFIN produces a binary measure of whether a particular gene is expressed in a given tissue type. ZFIN also produces anatomy terms and labels that are added into the feature matrix. FIG. 16 shows Expression data 2134 and ZFIN Anatomy Terms 2135 being extracted from ZFIN.org 2112 and being added to the feature matrix in the Concatenation step 2150. It should be understood that other databases may be used in place of ZFIN.org 2112, and Ensembl 2111 to aquire similar data types 2130, 2131, 2132, 2133, 2134, 2135, and 2136 in the feature matrix created in the Concatenation step 2150.

RNA-seq time series expression data 2120 is gathered from White et, al. 2110 to provide information on whether a gene is expressed and to what degree it is being expressed (gene encoded in DNA being transcribed to RNA before expressing into a protein) at a particular time point. For example, if a gene is never expressed, this might be informative to a machine learning model to suggest this gene may have no role in zebrafish biology and its deletion may not result in an observable phenotype and if a gene is highly expressed through all stages of development, the model may infer this as a highly essential gene and predict severe phenotypes as a result of gene disruption (White, R. J., et al., A high-resolution mRNA expression time course of embryonic development in zebrafish). The RNA-sequence time series gene expression data 2120 is compiled and concatenated in the concatenate step 2150 with the Ensembl 2111, ZFIN.org 2112, and ProteinBERT 2140 data pertaining to each gene into the feature matrix. It should be understood that the concatenate step 2150 can be performed without all of the data types, and a feature matrix can still be created and used without all of the information from White et al. 2110, Ensembl 2111, ZFIN 2112, and ProteinBERT 2140 and with information from other databases or deep/machine learning models.

In some embodiments, features pertaining to the genes are filtered after the concatenation of the data relating to individual genes. Features below a certain variance threshold are filtered out to remove outliers in the data and missing or broken Boolean features are added using simple imputation (assigning values based on inferences to NULL or missing data). The features also go through feature scaling/data normalization to standardize data pertaining to the features of each gene. The new data, now normalized, is funneled into the model selection and model training.

In FIG. 16, Feature selection 2150 is performed on the data held in the feature matrix at the Concatenate step 2150. Low varience features are removed from the data, and imputation and scaling of the data if performed. The now optimized data is moved to Model Selection and Model Training 2152.

At the Model Selection and Model Training 2152, the hyperparameters of these models are tuned to produce a more realistic outcome. The hyperparameter tuning is conducted on a per-label (per gene) basis such that a held out test set is first generated using a stratified split consisting of 20% of the overall test set. Within the training set, a Bayesian hyperparameter tuning experiment is conducted with a total of 100 trials evaluated using 5-fold cross validation. The best hyperparameters are chosen from these trials and then used to retrain on the entire training dataset.

The model performance is evaluated on the held out test set.

In a preferred embodiment, the hyperparameters are chosen using Bayesian optimization. Bayesian optimization can be used to build a probabilistic model of the function mapping from hyperparameter values to the objective evaluated on a validation set. This technique has been shown to generate better hyperparameters using fewer trials than random and grid search. One of ordinary skill in the art would understand that random and grid search methods would still be useable for determining hyperparameters.

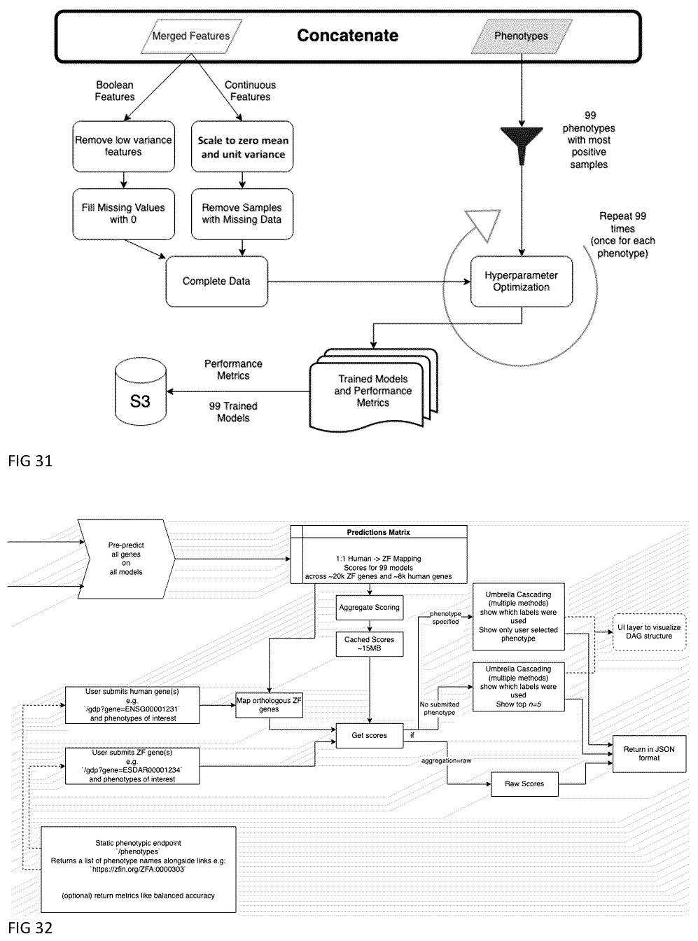

FIG. 31 is a more in-depth diagram of FIG. 16 illustrating the method for model training for the phenograph and gene-disease prioritization machine learning models. The features merged at the concatenate step (GO, KEGG, Protein Domains, Orthology, ZFIN expression, protein sequence information, etc.) at separated into Boolean features (accepts 1 or 0 values pertaining to TRUE or FALSE) and continuous features (accepts all other data types). The low variance Boolean features are removed from the data and the missing or NULL values are replaced with a 0, or FALSE value. The continuous features are scaled to the mean of zero with a unit variance of 1, and the samples with missing data are removed from the selection. This allows the scale variation to be even acoss all features. This also provides more accurate data for the model to be trained with, and in turn provides more accurate results when using the model on real test data. Once the training data is completed, it moves to the hyperparameter optimization/tuning step. The phenotypes with the most positive samples are added to the other training data at the hyperparameter optimization step, and the model is tuned to fit the training data. The new model is trained with the introduction of each new phenotype, and the hyperparameters are optimized for each new model. The trained models and performance metrics of those models are added to an S3. There will be a trained model created for each phenotype. The embodiment illustrated by FIG. 31 includes a model being created for each of the 99 most positive phenotypes, but more models could be created.

Other embodiments may include a non-equal ratio of phenotypes to trained models. Other embodiments may store the trained models and performance metrics in separate databases. The data may also be stored in other types of public, private, cloud, centralized, decentralized, etc. databases for storage. The data in the database is stored and may be used on real test data to determine new gene-phenotype, drug-phenotype, and disease-phenotype associations.

FIG. 32 is a more in-depth diagram of FIG. 16 illustrating the aggregate scoring of genes to phenotypes and diseases, and the production of a gene-disease association score. After the feature selection 2151 and model training 2152 (as shown in FIG. 16) are sent to the S3, an algorithm is used to Pre-predict all genes on all models 2156. In FIG. 32, the models are used to predict which genes cause certain phenotypes and the human genes are mapped with zebrafish genes in a prediction matrix containing scores for each model pertaining to individual phenotypes.

In other embodiments, human genes may be mapped with mouse genes, other human genes, or genes belonging to other animals that share human orthologs. This data may be collected from databases, patient electronic medical records (EMRs) and electronic health records (EHRs), lab tests and experiments, or in-vivo clinical trials.

After the human-zebrafish mapping, an aggregate score is created by using the trained models to predict the relationships between human diseases, human genes, zebrafish genes, and zebrafish phenotypes. A user may enter human genes of interest to map the gene to a zebrafish gene. An algorithm is used to get an association score for that zebrafish gene mapped to certain human diseases. In some embodiments, the user may submit a specific phenotype of interest to create a gene-phenotype score to be sent to the phenograph user interface.

In some embodiments, the association score is converted into a raw score in JSON to be sent to the phenograph user interface. Other embodiments may include formats other than JSON. Other embodiments may include association scores created using genes pertaining to animals other than zebrafish, such as mice or other animals that share human orthologs.

Certain problems such as overfitting and underfitting of can arise when working with machine learning models. Overfitting refers to a model that overfits to training data and may cause the ML model to perform very well in training, but poorly in a real environment. Overfitting happens when a model learns the detail and noise in the training data to the extent that it negatively impacts the performance of the model on new data. This means that the noise or random fluctuations in the training data is picked up and learned as concepts by the model. The problem is that these concepts do not apply to new data and negatively impact the models ability to generalize.

Overfitting is more likely with nonparametric and nonlinear models that have more flexibility when learning a target function. As such, many nonparametric machine learning algorithms also include parameters or techniques to limit and constrain how much detail the model learns.

In preferred embodiments, overfitting may be prevented in several ways. One way is if hyperparameter tuning is conducted entirely within the training set and the test set is only evaluated once using the final model which has been trained and tuned using the entirety of the training dataset Hyperparameter tuning is conducted using 5-fold cross validation. In practice, this means that for each set of hyperparameters, the training set is broken into 5 equal sized chunks. In each round of training, 4 of these chunks are used to train the model while the last is used to evaluate the model. This process is repeated 5 times for each set of hyperparameters and the average scores are used to help guide the Bayesian optimization process. Since the evaluation scores for each set of hyperparameters is generated from a held-out dataset (the last chunk of data), it is unlikely that over-fit parameters will generate strong scores. Thus, this training process limits the degree of overfitting.

After Model Selection and Model Training 2152 (as shown in FIG. 16), Performance Metrics 2181 of the models are sent to an Amazon S3 2154 for storage and the newly Trained Models 2182 are sent to another Amazon S3 2155 for storage. Other embodiments may include only a single S3 for storage of both the Performance Metrics and the Trained Models. Other storage services such as Google Cloud or Microsoft Azure may be used instead of Amazon S3. The data from the Feature Selection step 2151 and the Trained models 2182 are then sent to the Pre-predict all genes on all models step 2156. Here, the Trained Models 2182 are used to predict disease-gene-phenotype relationships based on the data gathered in the Feature Selection step 2151. This information proceeds to the Raw Prediction Look-up Table 2157 where the raw association scores for gene-disease relationships can are collected. These predictions are Aggregated 2158 and sent to the Phenotype Score Look-up Table 2159 where the scores are compared to possible phenotypes associated with the genes and diseases of interest. The raw scores and the aggregate scores are sent to the phenograph user interface 2160.

In a preferred embodiment, a custom evaluation metric is defined based on the accuracy metrics expected in wet-lab experimentation. Here, a selection of 20 genes is chosen for experimentation where only 1-2 are needed to be effective to move forward in the drug discovery process. As a result, the metric defined is inspired by those used in recommender systems where predictions are first ranked according to their prediction probability and then subsetted to the top-k elements. The only relevant accuracy is among these k elements. Relative risk (the proportion of true positive elements with respect the background prevalence) within the top 20 predicted genes was used to evaluate model performance.

The performance metrics of these models are sent to a simple storage service (S3) for storage. The trained models are sent to another S3 and all genes that were earlier concatenated are tested through all phenotype models to determine and predict if certain genes display certain phenotypes

To provide a conputationally efficient graph-based method for identifying subpopulations in high-dimensional single-cell data, the software may utilize a central database that links various biological nodes such as diseases, genes, phenotypes, drugs, and model organisms using edges derived from either experimental evidence or predicted via machine learning strategies such as GDP or phenolog mapping (as described above). Currently, this database consists of >200,000 nodes and >4M edges with plans to grow and expand. This database consists of a data layer, a python API layer and a front-end user interface.

In FIG. 16, The information from the Raw Prediction Look-up Table 2157 and the Phenotype Score Look-up Table 2159 are sent to the Phenograph 2160. A User 2170 may access the Phenograph 2160 their computer or other Device 2161 and enter Candidate Genes and Phenotypes of interest 2171 into the Phenograph Platform 2160 to access information about disease-gene-phenotype relationships and create Gene Shortlists 2162.

In a preferred embodiment, Training data is periodically updated (usually 1-2 times a year) to re-download data from ZFIN, Ensembl, and other sources. This updating frequency was chosen to balance time, cost, and efficiency of the model The full preprocessing stages have been automated so a script can be re-run to generate all the results requiring minimal human intervention. However, due to the number of hyperparameter tuning stages, this process takes ˜1 week of computational training time. With better processors, a shorter computational training time could be achieved. a shorter computational training time could also be achieved by using a smaller number of training iterations or less hyperparameter tuning.

Unlike typical machine learning platforms that need constant online predictions, the entire scope of possible predictions for GDP can be limited to the number of genes in the human genome with identified zebrafish orthologs. For this reason, features for these genes (as described in Data Gathering) are first gathered and then assigned a predicted probability score for each gene across all trained models for each phenotype resulting in a large prediction matrix relating thousands of human genes to hundreds of zebrafish phenotypes according to a continuous probability score. This matrix is then integrated into the phenograph where users can develop custom queries to identify genes of interest pertaining to certain phenotypes. These genes of interest may then be validated in physical experiments.

Phenologs are defined as an orthologous phenotype shared across organisms. For example, heart disease in humans may be represented as heart defects in zebrafish. However, these relationships are not always as trivial as the example above, where non-obvious relationships may exist between human diseases and model organism phenotypes. Mapping the phenologs of zebrafish and humans may provide information on testable genes and phenotype. The Phenolog Mapping Engine uses a gene-overlap strategy to identify phenologs and phenotype relationships between humans and zebrafish.

OpenTargets, a database for genes and variants, is used with Ensembl Gene IDs to find Human Disease and phenotype relationships. ZFIN is used with Ensembl Gene IDs to find zebrafish phenotype relationships. EggNOG, an orthology database that contains organisms, viruses, orthologous groups, trees, and algorithms, is used to map protein IDs to orthogroups (sets of genes derived from a single gene in a shared ancestor). Biomart is used to extract data from Ensembl to map Gene IDs to protein IDs. All of this information is processed, and human and zebrafish disease relationships, phenotype relationships, Protein IDs and Gene IDs are mapped based on human and zebrafish shared orthogroups. Based on this information, Human orthogroup-disease relationships and zebrafish orthogroup-phenotype relationships are determined. A hypergeometrical statistical test is conducted to identify if these relationships are just noted due to random chance. The output after this test determines the relationship between human disease and zebrafish phenotype. This information can now be used in physical experiments in relation to gene testing, phenotype testing, and testing of drug candidates.

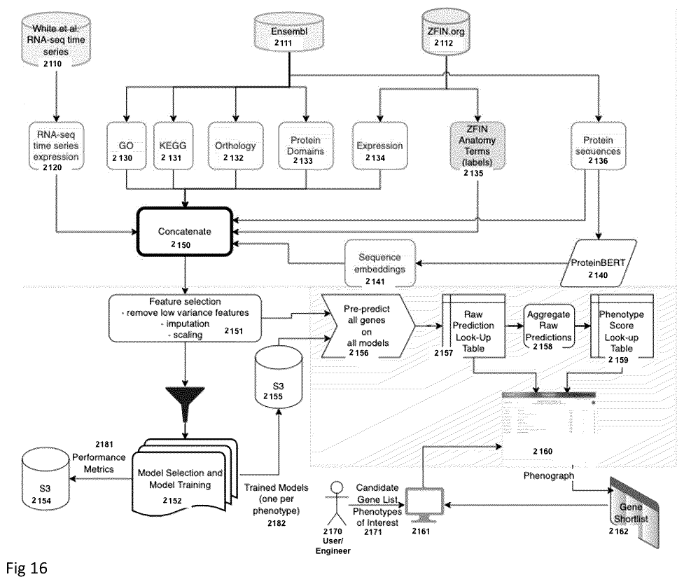

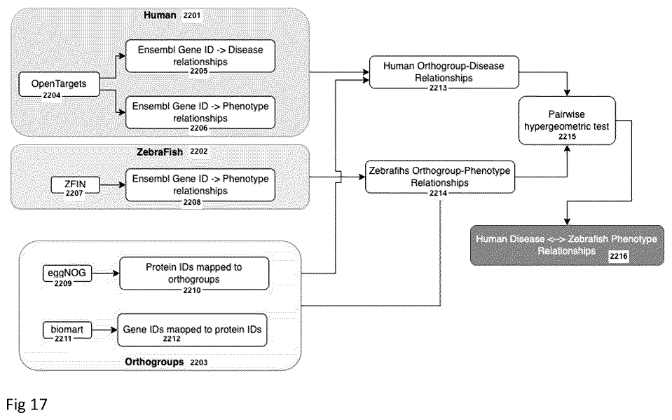

FIG. 17 depicts a method for producing human disease to zebrafish phenotype relationships. OpenTargets 2204 is used to obtain Human Ensembl Gene ID to Disease Relationships 2205 and Human Ensembl Gene ID to Phenotype Relationships 2206. ZFIN 2207 is used to obtain Zebrafish Ensembl Gene ID to Phenotype Relationships 2208. Protein IDs mapped to orthogroups 2210 is extracted from eggNOG 2209 and Gene IDs mapped to protein IDs 2212 is extracted from biomart 2211. Human information 2201 and Orthogroups information 2203 compiled into Human Orthogroup Disease Relationships 2213. Zebrafish information 2202 and Orthogroups information 2203 is compiled into Zebrafish Orthogroup-Phenotype Relationships 2214. A Pairwise Hypergeometric Test 2215 is done on the Human Orthogroup Disease Relationships 2213 and Zebrafish Orthogroup-Phenotype Relationships 2214 to create Human Disease to Zebraphish Phenotype Relationships 2216.

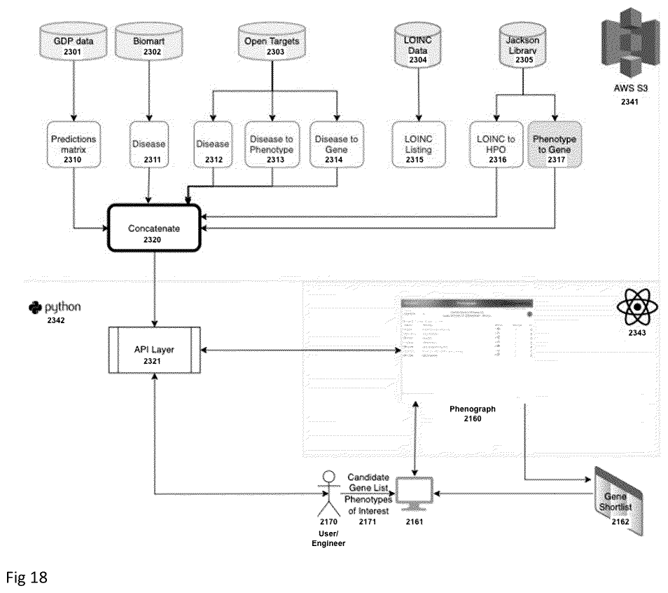

FIG. 18 depicts the connection of a feature matrix with an API layer that sends biological data to the phenograph. Predictions Matrix data 2310 from the Gene Disease Prioritization (GDP) data 2301, Disease data 2311 from Biomart 2302, Disease data 2312, Disease to Phenotype relationships 2313, Disease to Gene relationships 2314 from Open Targets 2303, LOINC Listings 2315 from LOINC data 2304, and LOINC to human phenotype ontology (HPO) 2316 and Phenotype to Gene relationships 2317 from Jackson Library 2305 held in AWS S3 2341 are concatenated in the Concatenate step 2320. Other embodiments may include data stored in other cloud environments or in local databases and biological data extracted from other sources, such as ZFIN or Ensemble data.

This data is sent to an API Layer 2321 run using Python 2342. Some embodiments include a REST or SOAP API layer run using languages such as Python, JavaScript, Ruby on Rails, or JAVA in an XML or JSON format. Other APIs, languages, and formats may also be used. User 2170 may be a developer that can access the data directly from the API Layer 2321. The concatenated data is sent from the API Layer 2321 to the Phenograph 2160. Phenograph 2160 is the same phenograph as in FIG. 16. Other embodiments may include a Phenograph 2160 that is not the same as the one in FIG. 16.

User 2170 may access the information held on the Phenograph 2160 using Device 2171. The web application that most Users 2170 use to access The Phenograph 2160 is built using a React Javascript Library 2343. Some embodiments may include a user interface built using a language other than React JavaScript. The user may also use the Phenograph 2160 to create Gene Shortlists 2162 containing their genes of interest.

### (13) Phenograph Development Environment

The phenograph is split into two environments, a Local Development Environment, and a Deployment (deployed) environment. The Local Development Environment is used for the development of new features of the application, and therefore is not needed to be a constantly running environment. The Deployed Environment is executed via a Docker image running on AWS Elastic Container Service (ECS) using AWS Elastic Compute Cloud (EC2) web services. Bitbucket is used to fully automate the deployment of source code to AWS EC2 servers. Bitbucket helps to store, manage, track and control code and changes to code similar to GitHub.

In a preferred embodiment, the Phenograph is backed by an SQLite database with separate tables for the different types of nodes and edges in the graph. These nodes may pertain to conditions such as diseases and phenotypes, human genes, zebrafish genes, and lab tests. The edges in the graph pertain to the other tables, where the unique identifier is a pair of foreign keys. Some edge tables contain additional data besides the foreign key copied from another. This data replication simplifies the joins required for high frequency access patterns.

The edges connect a pair of nodes in an undirected manner. For example, the gene node BRCA2 is connected to the disease node “breast carcinoma”. All human genes are stored in a gene table (“hum_genes”), all diseases are stored in a separate disease table (“conditions”). The connection between genes and diseases are stored in an edge table called “condition_to_gene”. Attributes describing each connection such as the score and the evidence count describing the strength of the gene-disease association are also stored in the edge table. Similar edges exist between diseases to other diseases, diseases to phenotypes and phenotypes to clinical lab databases (LOINCs).

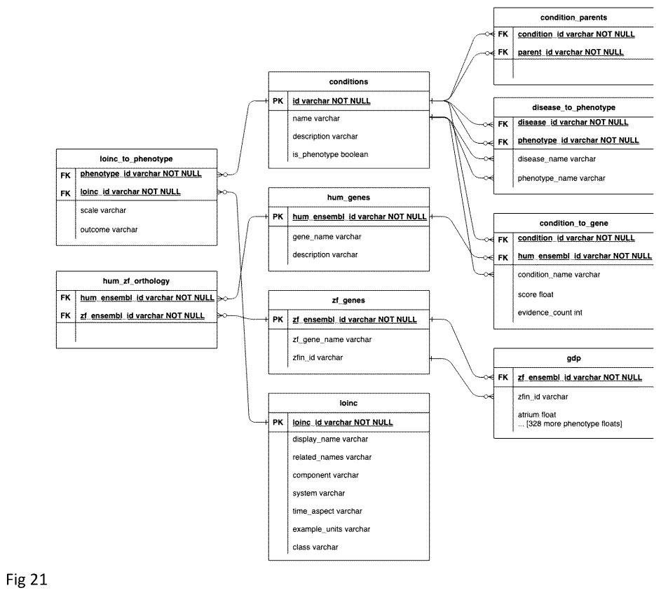

FIG. 21 is a diagram of the Phenograph database Schema. The primary data types are shown in the middle column. These primary data types pertain to nodes in the phenograph. In example embodiments, these primary data types may be conditions, human genes, zebrafish genes, and LOINCs (lab tests). These nodes use “varchar” to hold information in strings. The information may be names, descriptions, genes, phenotypes, ID, display names, related names, components, etc. In other embodiments, the primary data types (nodes) may pertain to other data types such as information about other organisms (rats, mice, mammals, insects, fish, ect.) and these strings may include other information displayed by the phenograph.

The edges in the phenograph pertain to the other tables in FIG. 21, loinc_to_phenotype, hum_zf_orthology, condition_parents, disease_to_phenotype, and condition_to_gene. The unique identifier for these edges is a pair of foreign keys used to refer to a primary key in another table. These keys allow for the relation of data from multiple tables and between nodes. This allows for the relationship of data such as gene-disease relationships, gene-phenotype relationships, disease-phenotype relationships, human orthogroup-disease relationships, zebrafish orthogroup-phenotype relationships, human-zebrafish disease-phenotype relationships, etc. These foreign keys also ensure data integrity by preventing the removal of integral data in a table that is being used by another table. The “varchar” for the IDs stored in these tables are “NOT NULL” meaning they cannot store null data, or data that contains no value. These foreign keys are shown as “FK” in FIG. 21, and they are pulling the primary key, or “PK” or the table that the “FK” is connected to. This allows for the movement and connection of data between the tables. The phenograph uses this data when displaying the relationships between disease, phenotype, genes, etc. to the user. These relationships pertain to copied data fields shown with arrows in FIG. 21. Other embodiments may include proteins, drugs, chemicals, etc. or other identifiers or descriptions providing insight on biological or chemical data.

The GDP table shown in FIG. 21 contains floats pertaining to zebrafish phenotypes. This data is related to the “zf_genes” table for zebrafish genes, names, and identifiers. Floats are used to store the phenotype data because of the large amount of data needed to be stored. Floats allow for a significant amount of data to be stored as numbers while keeping the storage space needed for that data at a reasonable size. This data can be related to human/zebrafish orthology to then be related to human genes. This provides relationship information between human genes and zebrafish phenotypes that can then be used in clinical trials or other lab experiments. This information may be displayed to the user of the phenograph and may be filtered to contain only information regarding candidate genes, diseases or phenotypes relevant to the user.

The SQLite database containing separate tables for nodes and edges is housed on S3, with all past versions kept and timestamped, so that the results from a given Phenograph version can be easily checked or reverted to. The Phenograph is run, either locally or deployed, by copying the preferred version housed on S3 to a local machine or instance. The database is read-only for the life cycle of the running Phenograph. Whenever new public or proprietary data becomes available, the database is updated to incorporate the new data into the Phenograph.

The phenograph deployed environment is a constant-running environment that may need to be updated to reflect new features or new information added to the local environment by developers. The phenograph is automatically updated first by running a DVC script to collect and download all our source datasets. This script also rationalizes and harmonizes all datasets and writes a new version of the SQLite database. The database is then version stamped and written to S3, and the Phenograph code repository is lagged with the new version stamp. All automated and manual testing is performed to ensure continued functionality, and a Pull Request (PR) is made for the version update. This branch and all subsequent versions of the Phenograph read the updated version stamp to know which version of the Phenograph database to copy from S3 (for example, during the Continuous Integration testing pipeline). Once the PR is reviewed and merged, the main branch of code is deployed with the new database version. The Phenograph is automatically redeployed, and users can see the updated data.

Phenograph development to add new features may not require data updating, and therefore the current version of the database is simply copied from S3 during the build-test-deploy cycle. When updating the Phenograph database, the normal build-test-deploy cycle is also used, but in this case the local branch also creates a new version of the database in S3.

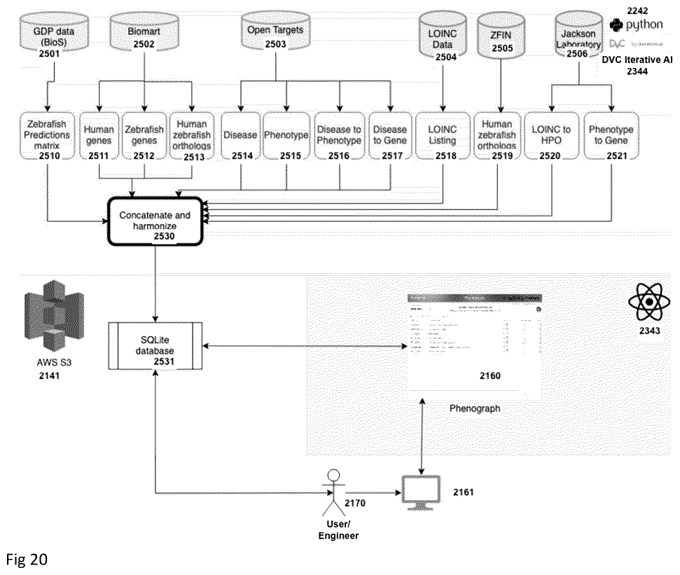

As shown in FIG. 20, Biomart 2502, OpenTargets 2503, Logical Observation Identifiers Names and Codes (LOINC) lab test data 2504, ZFIN.org 2505, and Jackson Laboratory 2506 databases are used to generate data for human genes 2511, zebrafish genes 2512, human/zebrafish orthologs 2513, diseases 2514, phenotypes 2515, disease to phenotype relationships 2516, disease to gene relationships 2517, LOINC listings 2518, Human Zebrafish Orthologs 2519, LOINC to human phenotype ontology (HPO) 2520, and phenotype to gene relationships 2521. This data is all Concatenated and harmonized using a data version control (DVC) pipeline at the Concatenate and Harmonize step 2530. The Zebrafish Predictions Matrix 2510, taken from the Gene Disease Prioritization (GDP) data 2501 is added to the other data types in the Concatenate and Harmonize step 2530. In other embodiments, not all of the information added is necessary, and information types from other databases may also be extracted and concatenated.

The Concatenate and Harmonize step 2530 puts all this information into an SQLite database 2531. Some embodiments may include minor cleaning steps such as storing all external IDs in a similar format (e.g., changing “HP_0001” from Jackson Laboratory to “HP:0001” to match the ID format from Open Targets). User 2170 may be a developer that has access to the SQLite database directly. The concatenated information is sent from the SQLite database to the Phenograph 2160 to be accessed by User 2170 on Device 2161. Phenograph 2160 is the same phenograph as in FIG. 16. Other embodiments may include a Phenograph 2160 that is not the same as the one in FIG. 16.

Some embodiments may include genes that have multiple names or IDs, such as an Ensembl ID or an Entrez ID. Data pertaining to different identifiers the same genes are concatenated to make up a single representation for each gene. This allows for the Phenograph to compile information of a single gene in one area even if the gene has multiple identifiers and allows for reduction of duplicate genes in searches performed by a user.

Once rationalized, the data is written to a local SQLite database and copied to the Phenograph S3 bucket. The bucket contains folders named for the date stamp of the database versions. A second file which gives the exact location on S3 of this new version of the Phenograph database is written locally. Any new versions of the Phenograph on this branch will pick up the new version of the SQLite file from the new location on S3. When this branch is merged to main, all new deployed versions will also use this new version.

Pytest is run to ensure that the new local database still passes the tests for compatibility and compliance, and any adjustments required for changes in the format of the source datasets are made. Once development is finished and the PR is accepted, the local data version control (DVC) cache is stored on S3. This produces a second versioning and caching system for extra restoration capability. The DVC cache is a binary format which is harder to work with than the human-readable SQLite.

DVC is an open-source data versioning software. In a preferred embodiment, DVC is used to manage different versions of the phonograph data. When DVC caches datasets, it stores it in a compressed binary format on a remote file storage system (Amazon S3). To access the cache and retrieve the data, the DVC software is used to pull from the cache and decompress the data. Using DVC also ensures that the version of data pulled from the cache is compatible with the current software version and ensures higher reliability.

This is automatically run as part of the Bitbucket pipelines when deploying to the production server. Because the deploy pulls from the S3 cache, it is not relying on all the original sources being available at the time of deployment. For example, if Open Targets goes down for maintenance, the Phenograph can still be deployed because it works from the cached version on S3. This also means that version updates from our sources won't break the Phenograph. For example, if one of the sources updates to a new version with a different schema, the deploys will still work from the old version until the problems are fixed.

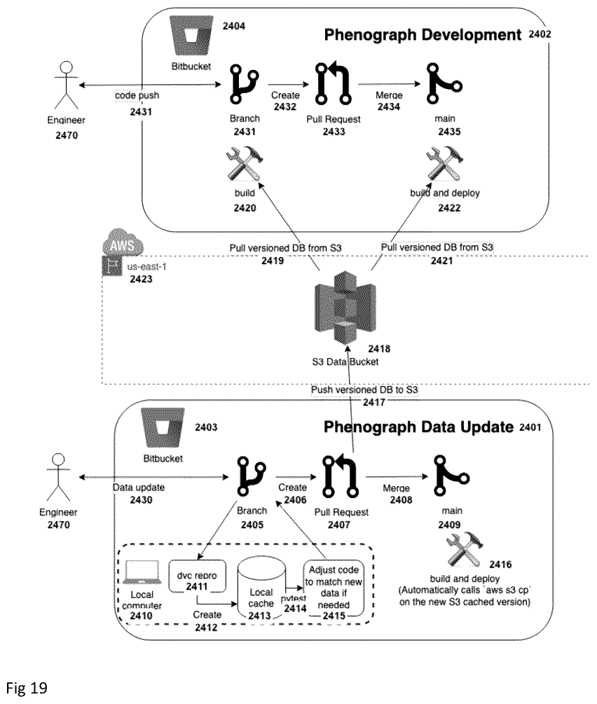

FIG. 19 is a diagram illustrating methods for adding features to and updating the data contained in the phenograph. The bottom path “Phenograph Data Update” 2401 depicts a method for updating the phenograph database. A User, Developer, or Engineer 2470 performs a Data Update 2430 to code in a bitbucket pipeline 2403. A Branch 2405 is taken from the main code 2409, and Created 2406. A Pull Request 2407 is opened to let other Developers or Engineers 2470 add to the code or see what has been added. After the new feature has been added, it is Merged 2408 to the master or Main Environment 2409. The Branch 2405 may connect with the DVC repro 2411 which saves the data files, intermediates, and final data in local cache 2413 on a Local Computer 2410. Pytest 2414 is run to ensure that the new local database still passes the tests for compatibility and compliance. The code is Adjusted 2415 to match new data if needed. This new version is Pushed 2417 to the S3 Data Bucket 2418 held on AWS 2423. In some embodiments, the Local Computer 2410 may be another device, and the AWS S3 may be another cloud based storage service. The Engineer 2470 may run Build and Deploy 2416 docker commands within the Bitbucket pipeline 2403.

The top path “Phenograph Development” 2402 depicts a method for adding new features to the phenograph using a Code Push 2431. This does not require data updating. A User, Developer, or Engineer 2470 pushes code to a bitbucket pipeline 2404. A Branch 2431 is taken from the main code 2435, and a new feature is added or Created 2433. A Pull Request 2433 is opened to let other Developers or Engineers 2470 add to the code or see what has been added. After the new feature has been added, it is Merged 2434 to the master or Main Environment 2435. A version of the S3 containing the Data Update 2401 may be Pulled 2419, 2421 to the Bitbucket Pipeline 2404 to either run Build 2420 or Build and Deploy 2422 docker commands within the Bitbucket pipeline 2404.

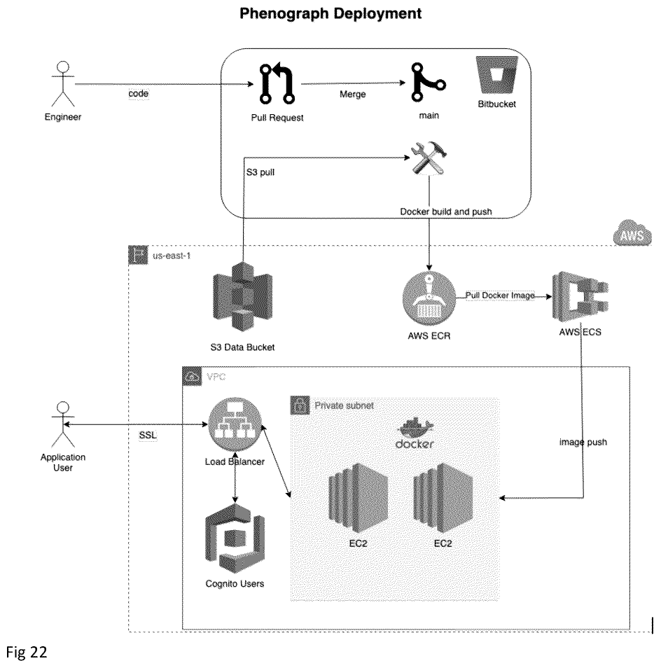

FIG. 22 is a diagram illustrating the phenograph deployment (deployed) environment architecture. An Engineer 2470 pushes code to Bitbucket and uses a pull request to merge the code to master or Main. The Engineer may also pull data from the S3 Data Bucket directly to start the Docker Build and Push to the AWS Elastic Container Registry (ECR), which is a docker container registry that allows for the storing, sharing, and deploying of docker container images. The ECR stored the code that has been written and packaged as a Docker Image. The AWS ECS then takes there files and actively uses them in the deployment of applications. In this case the application that is deployed is the updated Phenograph 2160.

AWS VCP allows for the launch of AWS resources into a virtual defined network. This network holds the Load Balancer, Cognito Users (user information such as ID and password), and the Docker EC2 instances that are being run on a Private Subnet with a route to an internet gateway that may be accessed by a User 2170 using their ID and Password credentials. A Secure Sockets Layer (SSL) is used to establish an encryption link between the User and the Load Balancer. The Load Balancer balances the number of Users 2170 and network traffic across the group of backend servers used to access the deployed Docker EC2 instances.

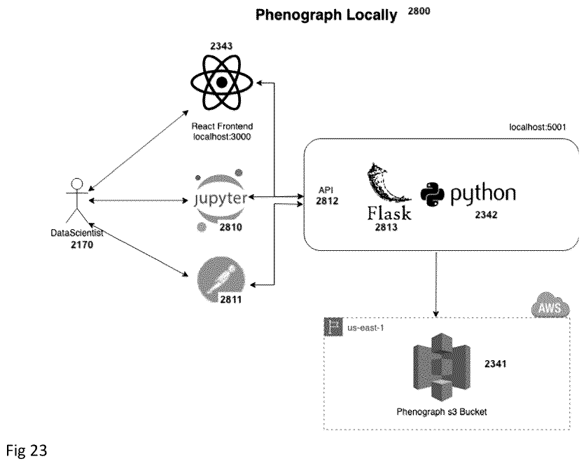

A data scientist can access the Flask (RESTfull) API using python via a terminal (jupyter). This API connects to the Phenograph S3 Bucket held on AWS. FIG. 23 is a diagram illustrating the phenograph local development environment architecture. A User or Data Scientist 2170 may access the phenograph locally using React JavaScript on the Frontend or use Jupyter 2810 to access the Flask API 2813 or Python API 2342 localhost environment. Postman 2811, an API testing platform, may also be used to directly access the Flask API 2813 or Python API 2342 localhost environment the without the need for the React frontend library 2343. The Phenograph S3 bucket 2341 may be accessed on the frontend or through the API localhost environment.

### (14) Phenograph User Platform

The Phenograph user platform is a platform for users to access all of the data, information, and tools available in the phenograph. The phenograph information including but not limited to phenotypes, genes, gene ontology, genome sequencing, orthology, protein data, high dimensional single-cell data, biological relationships, gene-phenotype relationships, gene-disease relationships, or other biological data. Users can use the platform to find and analyze data, create and save custom data sets, and share data sets with other users.

In a preferred embodiment, users can do searches such as disease name to disease ID, disease ID to gene, disease ID to phenotype, gene symbol to phenotype, phenotype ID to gene, phenotype ID to LOINCs, and lab name to LOINCs. These searches use a primary search type, such as disease ID, disease name, gene symbol, and phenotype ID, to output an identifier for a preferred output type, such as disease ID, gene, phenotype, or LOINCs. Users can input a primary search term to generate a listing of all possible relationships between the primary search term and preferred output type.

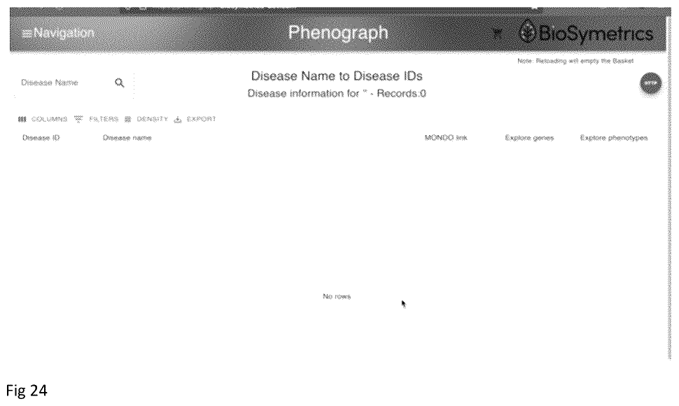

FIG. 24 is an image of the phenograph with the navigation tab, search bar, filters, and other features. This figure also shows the Disease name to Disease ID information accessed from the navigation tab.

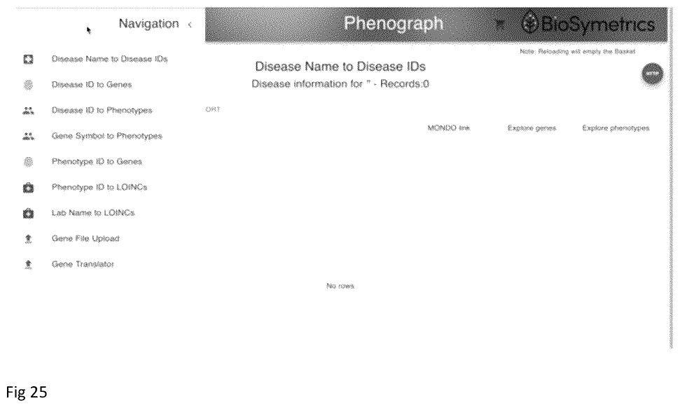

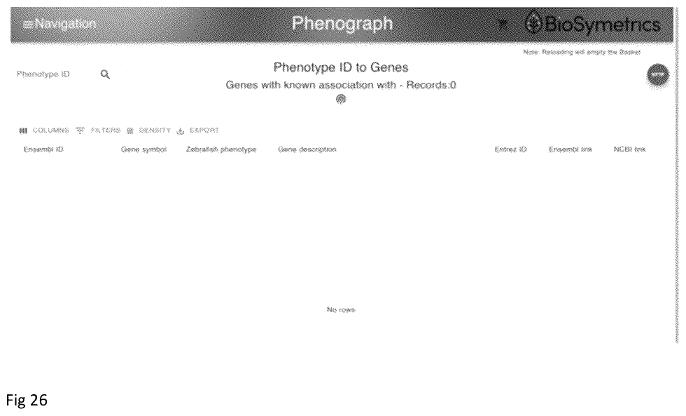

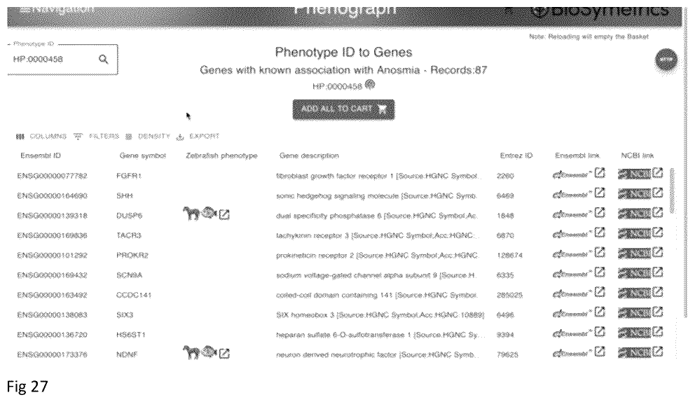

FIG. 25, displays a user view of the navigation tab in the phenograph. A user may click on disease name to disease ID, disease ID to gene, disease ID to phenotype, gene symbol to phenotype, phenotype ID to gene, phenotype ID to LOINCs, and lab name to LOINCs, to access a search page for their desired type. If a user clicks on “Phenotype ID to Genes”, the screen in FIG. 26 is displayed. In this embodiment, a user may provide the search bar with a desired phenotype, such as anosmia or loss of smell, and a phenotype ID will be generated (the phenotype ID for anosmia is HP-0000458). Users can use that phenotype ID to search for diseases and genes that pertain to that phenotype. The diseases and genes will be compiled and shown to the user in a list. The list compiled for HP:0000458, or anosmia, is shown in FIG. 27. FIG. 27 shows genes that relate to the anosmia phenotype and these genes are listed with Ensembl ID, Gene symbol, Zebrafish phenotype, Gene description, Entrez ID, and links to Ensembl and NCBL. Users have to option to export the list or filter the list in terms of other specific phenotypes. The zebrafish phenotype box displays whether or not the phenograph can predict a zebrafish phenotype for that gene. The links to Ensembl and NCBL provide a way for users to get more in-depth information on the genes listed. The filters may also filter out genes that are associated with many other phenotypes not related to the phenotype of interest because these genes are more likely to have been tested on previously. For example, if a gene relates to anosmia, but very few other phenotypes, that gene would be a higher priority gene for investigation than one that associated with hundreds of phenotypes.

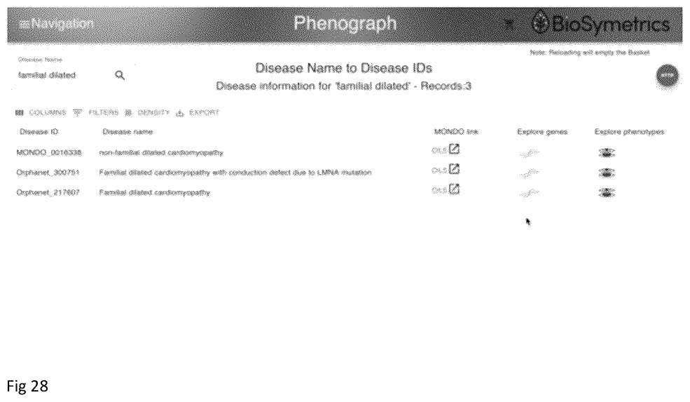

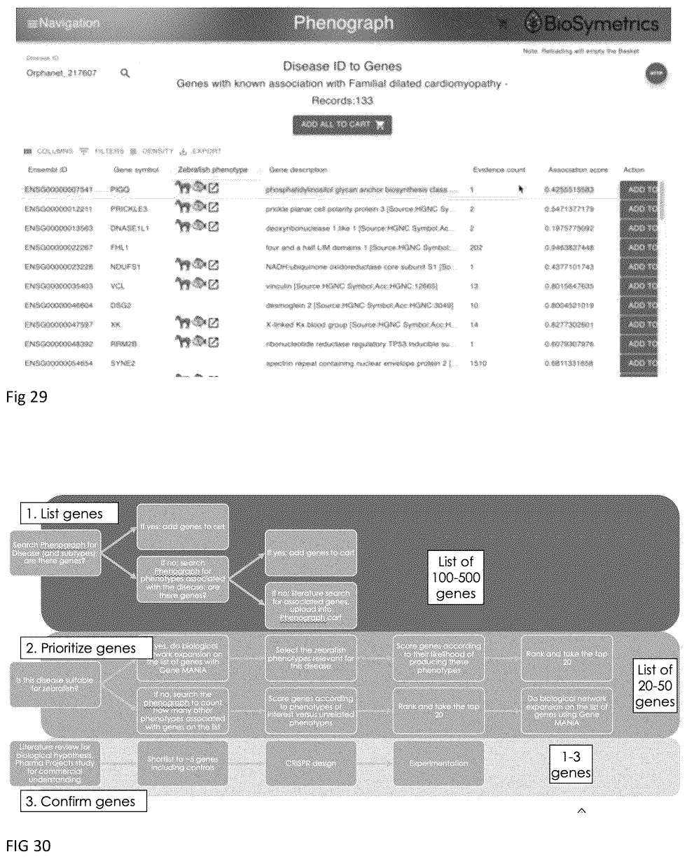

In some embodiments, a user may use a disease name to explore associated genes to that disease. In some embodiments, a user may also include only a part of the entire disease name. The AI will determine the disease that the user is most likely looking to search for and display search results pertaining to that disease. If there are multiple diseases that share the inputted partial disease name, all of the diseases are listed, and the user may pick one, some, or all of the diseases to perform the search on. FIG. 28 shows an image of the “Disease Name to Disease IDs” page after searching for the partial search term “familial dilated”. The search results come up with multiple diseases for the user to choose from when proceeding with the search. Links are also provided to allow users to learn more about each disease before choosing which ones they would like to focus on. Under the “Explore genes” column, users may click the double helix icon to perform a disease ID to genes search pertaining to the disease chosen. This search brings the user to the “Disease ID to Genes” page shown in FIG. 29, where a list of genes relating to the chosen disease is displayed. This figure shows that there are 133 genes with known association to Familial dilated cardiomyopathy. The phenograph also creates “association scores” for disease to gene relationships to allow genes that are more likely to cause diseases to be prioritized. Users can export this data or filter the data to show only genes with a higher association score. Users may use the “Add to cart” function of the phenograph to compile certain genes, phenotypes, and diseases of interest to one list even if they were found in different searches or pages.

After the user performs a search, all of the associated genes are compiled into a list available to the user. A GDP may be performed to shortlist the genes into good candidates to take to experimentation in a model organism.

In other embodiments, the phenograph may include additional features such as phenolog mapping, Integration with Gene MANIA, Aggregation of multiple zebrafish phenotypes to give an aggregate score, Ranking of genes by human phenotype sensitivity and specificity, integration of additional datasets such as drug candidate databases, and additional databases that provide more information on already existing categories in the phenograph.

FIG. 30 is a flowchart of the typical drug discovery workflow with the use of the phenograph. The flowchart illustrates the steps that a user would take from using the phenograph to experimentation. The user first searches through the phenograph for a target disease. If there are genes associated with the target disease, then the user may add those genes to the cart. If there are no known genes associated with the target disease, the user should search for phenotypes associated with the target disease. If there are genes associated with the target disease phenotypes, then the user may add those genes to the cart. If there are no genes associated with the disease or phenotype of interest, the user may perform a literature search for specific genes and add them to the cart. The user may choose to create a list of genes held in the cart and may filter them out using certain filters.

All genes added to the cart proceed to step 2, prioritize genes. If the disease is suitable for zebrafish (if the disease pertains to phenotypes seen in zebrafish), a biological network expansion may be performed on the genes using a biological network expansion software such as GeneMANIA. Zebrafish phenotypes relevant to the target disease are determined, and genes found with the phenograph are given a score based on the likelihood of producing that zebrafish phenotype. The user may choose as many genes as they would like to move to step 3. In FIG. 30, the user depicted chooses the top 20 genes.

If the disease does not relate to a phenotype found in zebrafish, the phenograph may be used to determine similar phenotypes associated with the genes added to the cart. The genes are then scored according to the number of phenotypes of interest and the number of unrelated phenotypes that relate to each gene. After determining the genes with the top association scores, a biological network expansion is performes using a biological network expansion software such as GeneMANIA.

After determining the genes with the top association scores, a literature review may be performed to create a biological hypothesis to use for gene experimentation. These genes may be edited using CRISPR, and in vivo experimentation may be performed to test these genes in labs or clinical trials. This experimentation may include the use of small molecule screening to determine the effects of certain drug-gene interactions.

Other embodiments may include a phenograph with data pertaining to mice or other animals that share human orthologs. It should be understood that a method of drug discovery without all of these steps would be possible.

### (15) Small Molecule Screening Library and Mechanism of Action Engine

Small molecule screening is a procedure in which small molecules (typically organic compounds with a molecular weight under ˜1,000 g/mol) are systematically tested for their ability to activate, perturb, or modify a target or a biological process of interest. Because of their low molecular weight, these molecules can enter cells more easily than larger molecules. These small molecules may enter cells and alter, destroy, or remove enzymes, proteins, nucleic acids, or smaller products of the cell such as metabolites from within the cell. Small molecule screening is used to predict drug candidates for various diseases, disorders, and conditions.

The term small molecule, which is used herein interchangeably with the terms small molecule compound, and drug, refers to a compound either synthesized in the laboratory or found in nature, which is typically characterized in that it contains several carbon-carbon bonds, and has a molecular weight of less than 1500, although this characterization is not intended to be limiting for the purposes of the present invention. Examples of small molecules that occur in nature include, but are not limited to, taxol, dynemicin, and rapamycin.

Some embodiments include generating a small molecule screening library using target gene and phenotype data as the starting point. This data is collected from clinical insights, the phenograph, and the Gene Disease Prioritization platform (GDP). Small molecules may be used in clinical trials or lab tests to determine their effect on genes, proteins, and cells.

In some embodiments, CRISPR is used to alter specific target genes, and a CRISPR assay is performed on these genes. The CRISPR-Cas system detects required DNA or RNA sequences using a disease diagnostic platform (in this case the phenograph). The genes with the top association scores are validated using transcriptomics (the study of RNA molecules in a cell).

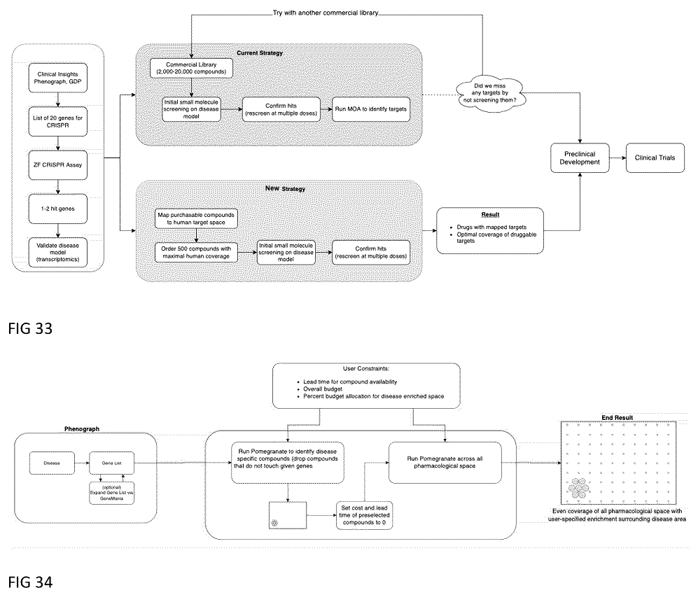

FIG. 33 is a diagram illustrating a small molecule screening process for drug development. Clinical insights, phenograph data, and GDP data are collected and a small list of genes is determined to be associated with a certain disease. CRISPR is used on these genes and a few “hit genes”, or genes with the biggest phenotypic variation are determined. Transcriptomics is used to validate the gene-disease association. The “hit genes” are tested on using a small molecule screening method. A commercial library (database) is used to gather compounds for the screening. Some embodiments may include multiple libraries, or a private compound library for use. An initial small molecule screening is performed on the disease model, and molecules that show promise are rescreened at multiple doses to confirm their eligibility. An MOA is run to identify targets. Other commercial libraries containing different small molecules are run through the same process. The small molecules that performed well in the screening are sent to preclinical development and eventually clinical trials.

Another method of small molecule screening includes mapping purchasable compounds to human target space (the bottom path in FIG. 33). The targets that have the widest range of coverage for human disease are noted and ordered. An initial small molecule screening for these compounds is performed, and likely candidates for drug-gene interactions are compiled. The likely candidates are mapped with target genes and an overview of the optimal coverage of the gene targets is generated. The small molecules then move on to preclinical development and eventually clinical trials.

In the context of drug discovery, there are traditionally two major schools of thought, target-based and phenotypic-based drug screening. In the former, an individual protein target related to the disease of interest is chosen and then screened against in isolation in very high throughput screens (e.g. thousands-millions of compounds). However, due to the complexity of biological systems, compounds that are effective on the target in isolation often fail when introduced to the whole organism. For this reason, phenotypic screening approaches (especially those conducted in vivo) offer a more reliable discovery strategy despite offering lower throughput (e.g. hundreds-thousands of compounds). This statement is not meant to teach away target-based screening as target-based screening is relatively useful when dealing with larger datasets or less complex drugs or diseases. Empodiments in the present disclosure may also combine the methods of target-based and phenotypic-based screening to target compounds at a lower price and faster than phenotypic-based screening can achieve alone.

FIG. 34 is a diagram illustrating a small molecule screening software and method of screening. The phenograph collects prioritized genes based on the input of a disease or disease ID. GeneMANIA or other similar network expansion software may be used to expand the gene list. Screening software is run to identify given small molecules able to control or modify the given genes. The software intakes certain parameters to gain insight about the possible compounds to use. These parameters may include the company or researcher's budget, the time before the compound is needed, the disease, the database library size or how many compounds are being screened, the known effectiveness of the drug, side effects, price of the drug, availability of the drug, etc. These are the main parameters used in the drug screening process, but many other factors may be included in the screening. Based on these parameters and information in public or private datasets containing drug-target data (price, availability, uses), a screening library is created that provides the user with compounds that are under or at budget and provide an optimal biological target diversity. Other molecules that are unable to affect the given genes are removed from the testing. An end result containing even coverage of all pharmacological space with user-specified enrichment surrounding the disease area is created.

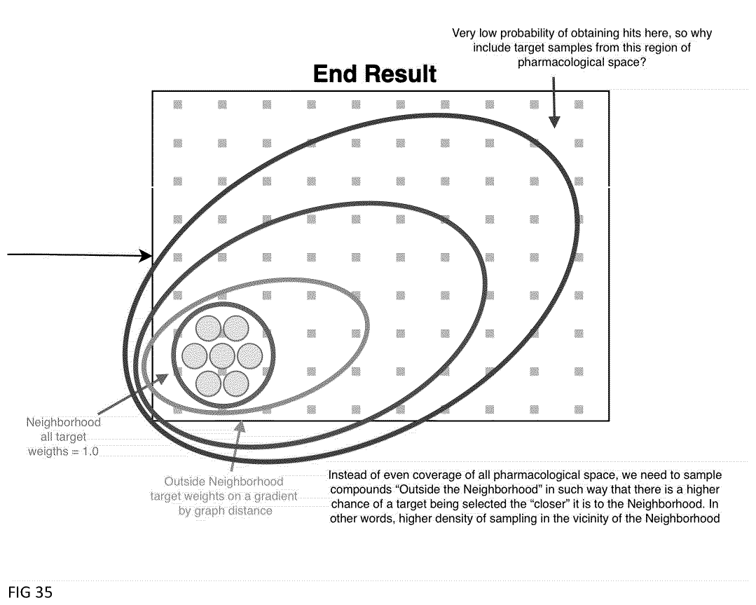

FIG. 35 is an illustration of the probability result after running the small molecule screening. This figure contains an in-depth look at the end result of FIG. 34 containing even coverage of all pharmacological space with user-specified enrichment surrounding the disease area.

In a preferred embodiment, the screening software's goal is to select a set of compounds from a library that will be optimal for experimentation. Each compound may have a set of features (including cost, availability, uses, drug interactions, etc.) that correspond to it. Each compound has a different set of features that may make it more or less compatible with experimentation. The software is used to maximize the number of positive features included in the list of testable compounds while reducing the time and cost of getting those compounds. Some of these features may be critical, and the software may weigh these features more than others. For example, the total price of the list must not be more than the overall budget for the experimentation. Other embodiments may include only some of the aspects found in the screening software or may also include more aspects that provide a better overall understanding of the compounds, the list, and the experimentation steps and processes. The software's goal overall is to weigh the maximum coverage of a list of drugs by cost, time, and other resources used to produce and/or acquire those drugs.

The screening software may include a library optimized for the disease area and overall coverage of disease-drug and phenotype-drug interactions. This allows the program to create a disease-enriched space for each disease or phenotype. This disease-enriched space provides a way to measure the overall coverage of a drug on the entire pharmacological space and provides insight on a list of drugs that may cover more space. This disease-enriched space created before measuring a drug's total activity on all pharmacological space is a step that is not used in a typical phenotypic drug library or phenotypic drug screening.

In a similar embodiment, the main variables taken into consideration are all linear and binary functions. The unknown variables and features may be weighted into a yes or no binary value of whether the compound should or should not be included into the list of compounds. The constraints may be linear functions including cost vs budget, the maximum number of compounds, etc. The objective of the drugs may also be cast as a linear function to provide an overall weight for each compound and the role that it performs in the given list compared to other compounds that may or may not be included in the list.

In another embodiment, the software may be run to provide faster results at the cost of a small amount of accuracy. This method of running the software may be incredibly useful when dealing with a larger data set than can be handled with the slower but more accurate method of running the software.

In another embodiment, the use of these binary and linear functions may be used to create integer values for each variable or feature in each compound. Integer programming refers to a set of methods used to solve constrained optimization problems where variables must be integers. When these values are either linear or integer values, an integer linear programming optimization method may be used to solve constrained optimization problems.

Various solvers and techniques have been developed to solve constrained optimization problems and many efficient numerical packages have been written including the python package PuLP. The process of solving an optimization problem can be broken down into getting the problem description, formulating the mathematical program, solving the mathematical program, performing come post-optimal analysis, and presenting the solution and analysis. PuLP easily calls many solvers so that there is no need to enter a mathematical program into the solver software. Other methods to solve constrained optimization problems may also be used with the screening software including other modeling solvers or solvers that require inputs from mathematical programs. Other mathematical optimization methods or mathematical programming may be used other than integer programming when the datatypes are more complex or the data cannot be simplified in a way without the loss of important data. Mixed-integer programming may also be used for mathematical optimization of non-discrete variables.

Mechanism of action (MoA) describes the process by which a molecule, such as a drug, functions to produce a pharmacological effect. This may cause effects like cell growth, protein interactions, nucleic acid interactions, phenotypic events, etc. The most common MoAs include inhibition of bacterial protein synthesis, inhibition of cell wall synthesis, inhibition of enzymatic activity, alteration of cell membrane permeability, and blockade of specific biochemical pathways, but others may also be observed. Embodiments in the present invention may include these MoAs as well as all other currect and newly discovered MoAs. It should be understood that the present disclosure will also be useable with all other MoAs discovered in the future.

Typically, in phenotypic drug screening, a large number of compounds are tested to see which ones promote therapeutic effects. However, even if a drug is effective, a crutial componect of treating the disease and providing efficacy and safety when seeking FDA approval is understanding its MoA.

A preferred embodiment includes a method of drug targeting and small molecule screening that includes a method of determining the MoA of a drug being used on a target gene or with a target phenotype and the drug's ability to promote or reduce protein interactions. This method allows for a faster drug screening process by using machine learning (ML) models and algorithms to predict potential biological mechanisms and underlying phenotypic changes in chemical screens.

In the same embodiment, there may be a machine learning platform or MoA engine that is used to predict the MoA in certain drug interactions with genes, phenotypes, proteins, diseases, bacteria, cells, nucleic acids, enzymes, etc.

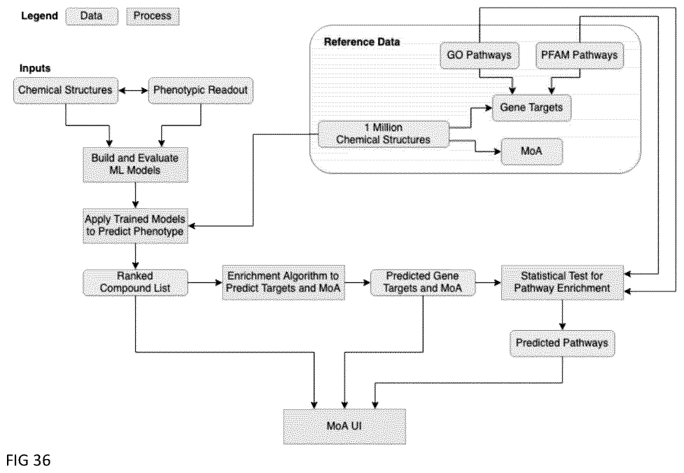

FIG. 36 is a diagram illustrating the steps taken by the MoA engine to provide insight on the MoA of certain drug-phenotype interactions. The MoA engine accepts a single CSV (inputs) file with two columns. The first column represents the structure of the chemical encoded in SMILES format. SMILES format is the “Simplified Molecular Input Line Entry System,” which is used to translate a chemical's three-dimensional structure into a string of symbols that is easily understood by computer software. Other formats such as chemical table files, Structure data format files, or other formats capable of displaying information useable by a computer or machine may be used instead of CSV. Other formats that display chemical information such as bonds and elemental information may also be used in place of SMILES format.

In the same embodiment illustrated by FIG. 36, the second column in the CSV file represents the activity label, denoting a binary value of whether the drug was effective or not effective in reducing phenotypic activity. Some embodiments may include other ways to display overall effectiveness of the drup, such as Boolean values (True/False), integer values (int) that include a score with the overall effect of the drug based on the signs or reduction in the severity of the displayed phenotype, string values, or other values that display the overall effectiveness of the drug on a given phenotype.

After collecting the Chemical structures and the Phenotypic Readout (the activity label), ML models are built and evaluated. Reference data (from databases) of chemical structures is collected and the trained models are applied to it to predict phenotypes that the specific structures would effect. A list is produced ranking the compounds based on their effectiveness on reducing a given phenotype and this data is sent to an enrichment algorithm to predict gene targets and MoA. A statistical test for pathway enrichment is performed based on gene ontology (GO) pathways and protein family (PFAM) pathways. These pathways and statistical tests are used to predict biological pathways that the drugs may take. The ranked compound list, predicted gene targets and MOA, and predicted pathways are sent to the MoA UI for users to view.

Typically, most phenotypic screenings do not have a binary endpoint, but instead either report a continuous measure or multiple features. Some embodiments may need to reduce the information of the screening to a binary value. When this occurs, a preprocessing strategy may be developed to convert complex inputs into a single binary measurement of activity. This output is the effectiveness of the drug on phenotypic activity.

Some embodiments may include the MoA engine being run with the screening software. This may include a better overall result when screening for specific molecules that may have MoAs that a user is looking for. The MoA engine may also be used to minimize the amount of target compounds tested by the screening software by limiting the compounds tested to only those that portray a certain MoA when dealing with target proteins, genes, phenotypes, diseases, or other compounds. The use of both the MoA engine and the screening software together may provide a faster, less costly, and simpler solution to the drug screening process than currently possible with other methods used in the industry.

### (16) Computer Systems for Implementing Various Embodiments

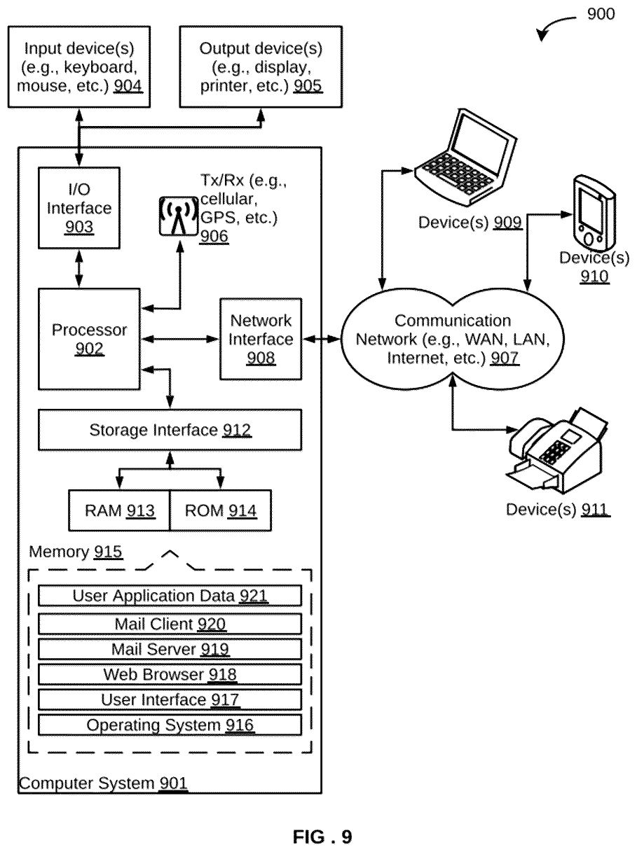

Referring now to FIG. 9, a block diagram of an exemplary computer system 901 for implementing embodiments consistent with the present disclosure is illustrated. Computer system 901 may include a central processing unit (“CPU” or “processor”) 902. Processor 902 may include at least one data processor for executing program components for executing user- or system-generated requests. A user may include a person, a person using a device such as such as those included in this disclosure, or such a device itself. Processor 902 may include specialized processing units such as integrated system (bus) controllers, memory management control units, floating point units, graphics processing units, digital signal processing units, etc. Processor 902 may include a microprocessor, such as AMD® ATHLON® microprocessor, DURON® microprocessor OR OPTERON® microprocessor, ARM's application, embedded or secure processors, IBM© POWERPC®, INTEL'S CORE® processor, ITANIUM® processor, XEON® processor, CELERON® processor or other line of processors, etc. Processor 902 may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), Graphical Processing Units (GPUs) (Nvidia, AMD, Asus, Intel, EVGA, and others), Tensor Processing Unites (Google), etc.

Processor 902 may be disposed in communication with one or more input/output (I/O) devices via an I/O interface 903. I/O interface 903 may employ communication protocols/methods such as, without limitation, audio, analog, digital, monoaural, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.n/b/g/n/x, Bluetooth, cellular (e.g., code-division multiple access (CDMA), high-speed packet access (HSPA+), global system for mobile communications (GSM), long-term evolution (LTE), WiMax, or the like), etc.

Using I/O interface 903, computer system 901 may communicate with one or more I/O devices. For example, an input device 904 may be an antenna, keyboard, mouse, joystick, (infrared) remote control, camera, card reader, fax machine, dongle, biometric reader, microphone, touch screen, touchpad, trackball, sensor (e.g., accelerometer, light sensor, GPS, gyroscope, proximity sensor, or the like), stylus, scanner, storage device, transceiver, video device/source, visors, etc. An output device 905 may be a printer, fax machine, video display (e.g., cathode ray tube (CRT), liquid crystal display (LCD), light-emitting diode (LED), plasma, or the like), audio speaker, etc. In some embodiments, a transceiver 906 may be disposed in connection with processor 902. Transceiver 906 may facilitate various types of wireless transmission or reception. For example, transceiver 906 may include an antenna operatively connected to a transceiver chip (e.g., TEXAS® INSTRUMENTS WILINK WL1283® transceiver, BROADCOM® BCM4550IUB8© transceiver, INFINEON TECHNOLOGIES® X-GOLD 618-PMB9800© transceiver, or the like), providing IEEE 802.11a/b/g/n, Bluetooth, FM, global positioning system (GPS), 2G/3G HSDPA/HSUPA communications, etc.

In some embodiments, processor 902 may be disposed in communication with a communication network 907 via a network interface 908. Network interface 908 may communicate with communication network 907. Network interface 616 may employ connection protocols including, without limitation, direct connect, Ethernet (e.g., twisted pair 50/500/5000 Base T), transmission control protocol/internet protocol (TCP/IP), token ring, IEEE 802.11a/b/g/n/x, etc. Communication network 907 may include, without limitation, a direct interconnection, local area network (LAN), wide area network (WAN), wireless network (e.g., using Wireless Application Protocol), the Internet, etc. Using network interface 908 and communication network 907, computer system 901 may communicate with devices 909, 910, and 911. These devices may include, without limitation, personal computer(s), server(s), fax machines, printers, scanners, various mobile devices such as cellular telephones, smartphones (e.g., APPLE® IPHONE® smartphone, BLACKBERRY® smartphone, ANDROID® based phones, etc.), tablet computers, eBook readers (AMAZON® KINDLE® ereader, NOOK® tablet computer, etc.), laptop computers, notebooks, gaming consoles (MICROSOFT® XBOX® gaming console, NINTENDO® DS® gaming console, SONY® PLAYSTATION® gaming console, etc.), or the like. In some embodiments, computer system 901 may itself embody one or more of these devices.

In some embodiments, processor 902 may be disposed in communication with one or more memory devices (e.g., RAM 626, ROM 628, etc.) via a storage interface 912. Storage interface 912 may connect to memory 915 including, without limitation, memory drives, removable disc drives, etc., employing connection protocols such as serial advanced technology attachment (SATA), integrated drive electronics (IDE), IEEE-1394, universal serial bus (USB), fiber channel, small computer systems interface (SCSI), etc. The memory drives may further include a drum, magnetic disc drive, magneto-optical drive, optical drive, redundant array of independent discs (RAID), solid-state memory devices, solid-state drives, etc.

Memory 915 may store a collection of program or database components, including, without limitation, an operating system 916, user interface application 917, web browser 918, mail server 919, mail client 920, user/application data 921 (e.g., any data variables or data records discussed in this disclosure), etc. Operating system 916 may facilitate resource management and operation of computer system 901. Examples of operating systems 916 include, without limitation, APPLE® MACINTOSH® OS X platform, UNIX platform, Unix-like system distributions (e.g., Berkeley Software Distribution (BSD), FreeBSD, NetBSD, OpenBSD, etc.), LINUX distributions (e.g., RED HAT®, UBUNTU®, KUBUNTU®, etc.), IBM® OS/2 platform, MICROSOFT® WINDOWS® platform (XP, Vista/7/8, etc.), APPLE® IOS® platform, GOOGLE® ANDROID® platform, BLACKBERRY® OS platform, or the like. User interface 917 may facilitate display, execution, interaction, manipulation, or operation of program components through textual or graphical facilities. For example, user interfaces may provide computer interaction interface elements on a display system operatively connected to computer system 901, such as cursors, icons, check boxes, menus, scrollers, windows, widgets, etc. Graphical user interfaces (GUIs) may be employed, including, without limitation, APPLE® Macintosh® operating systems' AQUA® platform, IBM® OS/2® platform, MICROSOFT® WINDOWS® platform (e.g., AERO® platform, METRO® platform, etc.), UNIX X-WINDOWS, web interface libraries (e.g., ACTIVEX® platform, JAVA® programming language, JAVASCRIPT© programming language, AJAX® programming language, HTML, ADOBE® FLASH® platform, etc.), or the like.

In some embodiments, computer system 901 may implement a web browser 918 stored program component. Web browser 918 may be a hypertext viewing application, such as MICROSOFT® INTERNET EXPLORER® web browser, GOOGLE® CHROME® web browser, MOZILLA® FIREFOX® web browser, APPLE® SAFARI® web browser, etc. Secure web browsing may be provided using HTTPS (secure hypertext transport protocol), secure sockets layer (SSL), Transport Layer Security (TLS), etc. Web browsers may utilize facilities such as AJAX, DHTML, ADOBE® FLASH® platform, JAVASCRIPT® programming language, JAVA® programming language, application programming interfaces (APIs), etc. In some embodiments, computer system 901 may implement a mail server 919 stored program component. Mail server 919 may be an Internet mail server such as MICROSOFT® EXCHANGE® mail server, or the like. Mail server 638 may utilize facilities such as ASP, ActiveX, ANSI C++/C#, MICROSOFT .NET® programming language, CGI scripts, JAVA® programming language, JAVASCRIPT® programming language, PERL® programming language, PHP® programming language, PYTHON® programming language, WebObjects, etc. Mail server 919 may utilize communication protocols such as internet message access protocol (IMAP), messaging application programming interface (MAPI), Microsoft Exchange, post office protocol (POP), simple mail transfer protocol (SMTP), or the like. In some embodiments, computer system 901 may implement a mail client 920 stored program component. Mail client 920 may be a mail viewing application, such as APPLE MAIL® mail client, MICROSOFT ENTOURAGE® mail client, MICROSOFT OUTLOOK® mail client, MOZILLA THUNDERBIRD® mail client, etc.

In some embodiments, computer system 901 may store user/application data 921, such as the data, variables, records, etc. as described in this disclosure. Such databases may be implemented as fault-tolerant, relational, scalable, secure databases such as ORACLE® database OR SYBASE® database. Alternatively, such databases may be implemented using standardized data structures, such as an array, hash, linked list, struct, structured text file (e.g., XML), table, or as object-oriented databases (e.g., using OBJECTSTORE® object database, POET® object database, ZOPE® object database, etc.). Such databases may be consolidated or distributed, sometimes among the various computer systems discussed above in this disclosure. It is to be understood that the structure and operation of the any computer or database component may be combined, consolidated, or distributed in any working combination.

It will be appreciated that, for clarity purposes, the above description has described embodiments of the invention with reference to different functional units and processors. However, it will be apparent that any suitable distribution of functionality between different functional units, processors or domains may be used without detracting from the invention. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

As will be appreciated by those skilled in the art, the techniques described in the various embodiments discussed above are not routine, or conventional, or well understood in the art. The techniques discussed above provide for preprocessing biomedical data for a predictive model using an ML algorithm. The ML algorithm uses different permutations of preprocessing parameters to generate an optimized preprocessing algorithm. The preprocessing of biomedical data is implemented via an AI/ML-based framework for big data analytics of biomedical data. The AI/ML-based framework also provides for an iterative feature selection module, a capability for integration of various datasets, and a parallel computing network. Various datasets are integrated, and the features are then selected for the combined dataset. The feature selection is optimized by another ML algorithm. The parallel computing network allows a plurality of users to work together on a same input data and can also be used to implement containerized deployment to execute the analytics at a faster rate.

The specification has described a method and a system for preprocessing biomedical data for a predictive model. The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope and spirit of disclosed embodiments being indicated by the following claims.

### (17) Zebrafish Imaging and Testing

Some embodiments may relate to the genetic testing of zebrafish using drug therapeutics testing, gene alteration, small molecule screening, and/or other biological testing on zebrafish genetics and phenomics. Genetic testing in zebrafish is often used to study the effects of genetic disorders such as cancer, heart defects, and neurological disorders. For example, researchers can use gene testing to study how mutations in certain genes can lead to the development of these diseases. Gene testing can also be used to study the effects of drugs or environmental toxins on the development of zebrafish. By studying the effects of these substances on the genetic makeup of the fish, researchers can gain insight into how they might affect human health.

Embodiments may contain methods of gene alteration, genetic engineering, gene transfer, gene targeting, and gene editing.

Gene alteration, or genetic engineering, is a process that allows scientists to modify an organism's genetic material, or DNA, to produce a desired trait or characteristic. The most commonly used method to alter the genetic makeup of an organism is through the introduction of foreign genetic material, or transgenes, into the organism's genome. This process, called gene transfer, can be accomplished through a variety of techniques, including genetically engineered bacteria, viruses, and transgenic animals.

Gene transfer using viruses is a particularly efficient method for introducing foreign genetic material into an organism's genome. Viruses can be adapted to carry and deliver a gene of interest into the genetic material of a host cell. Once the foreign gene is introduced, it may be expressed, or activated, in the cell, allowing the desired trait or characteristic to be expressed. This method is widely used in biotechnology and pharmaceuticals, as well as in agriculture, to produce plants and animals with desired traits.

Another method of gene alteration is gene targeting, which involves the use of recombinant DNA technology to modify or “knock out” a gene in an organism. Through this technique, a gene of interest can be silenced or removed from the organism's genome, resulting in the desired trait or characteristic being eliminated. This method is used in research laboratories to generate animal models and to study the effects of gene silencing on the organism's physiology.

In some embodiments, gene editing such as CRISPR may be used to alter zebrafish genetics, which allows scientists to directly edit the DNA of an organism. This is accomplished using engineered enzymes that can cut and paste a gene of interest into the organism's genome, resulting in the desired trait being expressed. This method is being used in research laboratories to study genetic diseases, develop new treatments and therapies, and even to create new organisms with desired traits.

Embodiments are intended to include or otherwise cover a variety of processes for determining phenotypes, genetic data, and other biological data of zebrafish. Some embodiments are for a zebrafish phenotyping platform to be used to determine alterations in zebrafish phenotypes based on a variety of factors. Embodiments may include methods of collecting zebrafish data including images, videos, and models, and processing these images, videos, and models using computer-implemented machine-learning algorithms to determine differences in zebrafish phenotypes.

Some embodiments may include bright-field imaging of zebrafish to determine alterations in phenotype data. These images may be compared to detect alterations of phenotypes between zebrafish during or after genetic testing.

Bright-field imaging of zebrafish is a technique used to view and analyze the internal structures of zebrafish embryos. It is a non-invasive imaging technique that uses light to create an image of the zebrafish embryo. The technique involves the use of light microscopy and a specialized light source without the need for fluorescent dyes. The light is directed onto the embryo, which is then imaged and analyzed. Bright-field imaging allows researchers to observe the development and growth of the embryo, as well as to study the structure and function of its organs and tissues.

Some embodiments may use bright-field imaging to observe the morphological changes that occur during embryogenesis and post-embryonic development, such as the formation of organs and tissues. It can also be used to study the expression of genes and their impact on development and behavior.

Bright-field imaging does not require special equipment or dyes, making it relatively low-cost and easy to use. It also does not require the use of toxins or radiation, making it a relatively safe imaging technique. Furthermore, since it is a non-invasive technique, it does not damage the specimens and can be used to observe them over long periods of time. Some embodiments may be used to observe the internal structures of the embryos, allowing researchers to gain a better understanding of the development and behavior of the embryo.

Some embodiments may concern a zebrafish phenotyping platform that provides the capability to screen both genetic and chemical perturbations in zebrafish and identify disease-relevant phenotypes. This platform may be used to identify zebrafish phenotypes using image and video data. To ensure this platform can perform at scale in an error-free manner, a data flow architecture that allows for efficient data processing and data access for end users is described.

Some embodiments may relate to gathering images, videos, and models of zebrafish from experimental facilities to upload to a zebrafish phenotyping platform. This platform may be used to determine the effects of various biological alterations (such as the altering of genes, or the use of drugs) on certain phenotypes of zebrafish. This may be useful for the testing of drugs for human diseases that are caused by human-zebrafish orthologous genes.

Some embodiments may also include fluorescent imaging of zebrafish. Fluorescent imaging of zebrafish is a powerful tool for studying the development and behavior of this species. It allows researchers to observe the behavior of the zebrafish in real-time and with great detail. Fluorescent imaging of zebrafish uses fluorescent proteins, such as green fluorescent protein (GFP) and yellow fluorescent protein (YFP), which are expressed in specific cells or tissues of the zebrafish. The fluorescent proteins are excited by a particular wavelength of light, and the light emitted from the excited proteins is detected with a camera or microscope. This allows researchers to visualize the behavior and development of specific cells or tissues of the zebrafish in real-time.

Computer processing methods may be employed in some embodiments to imitate fluorescent imaging using bright-field imaging techniques instead of fluorescent proteins. This allows certain areas to be illuminated as if fluorescence had been used, but eliminates the need for fluorescent dyes which may not be as reliable and could be more expensive.

### (18) Zebrafish Phenotyping Platform

In a preferred embodiment, the images of zebrafish are collected and scanned to determine and highlight specific organs of importance. A user may use the platform to determine the location, size, and shape of their organ of interest. This information may also be used to determine alterations in the phenotypes of the zebrafish in the image. A user may use the platform to collect data on a specific phenotype, such as an enlarged heart. The user may use the platform to automatically highlight the heart in every image after experimentation to determine which biological alterations used may cause an enlarged heart. Other users may look for other phenotypes such as loss of sight and highlight specific areas of the eyes in every image.

This platform may conduct tests on images and videos of live zebrafish using machine-learning models and algorithms. These images and videos may be collected from experimental/research facilities to be uploaded to the platform. Some models may include algorithms used to determine differences between zebrafish such as an enlarged heart, alterations in organ activity, differences in blood flow, differences in brain activity, etc. This information, as well as the images and videos, may be accessed by a user and used for research or informational purposes. This information may be stored in the cloud, on a host computer, in a database, or in any other user-accessible format.

In some embodiments, a user may upload their own images to compare with each other or with the images already accessible on the platform. These images may be stored in a database or other data/image-storing entity. Some embodiments may include machine learning models for the creation of segmentation labels for organs, systems, tissue, etc. As used herein, whenever “organ” or “organs” is used, it should be assumed that this encompasses organs, systems of organs, tissues, blood, or other biological entities. These labels with help highlight organs, systems, tissues, etc., of interest to allow for easy comparison without the need for manual labeling. These segmentation labels can be seen in the images in FIG. 40. Manual annotation of labels is possible after using the platform to label organs of interest. Some embodiments may include manually labeling all organs without the use of machine learning models. Some embodiments may include manual image annotations used as training data for the ML models. Once an image is annotated, this image may be fed back into the system to improve the models used by the platform.

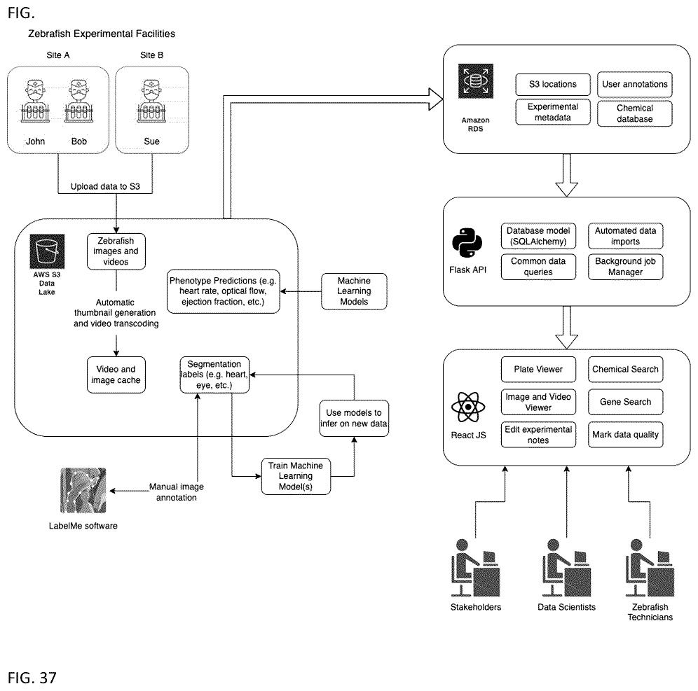

FIG. 37 is a schematic depicting the basic units of the zebrafish phenotyping platform. The zebrafish platform collects uploaded data, images, and videos from users. In some cases, these users collect this data directly from experimental/research facilities. The use of Site A and Site B is for showing that there may be more than one facility where information is collected. Some embodiments may include collecting data from a database, or only a single entity. Some embodiments may include collecting data from multiple databases or multiple other entities. Some embodiments may include collecting data from a combination of data sources.

Once the data, images, and videos are uploaded to the platform, they are sent to an AWS S3 Data Lake/Store. Some embodiments may include a platform that uses other cloud-based storage or local server storage. The platform takes the videos and generates a single image as the thumbnail that users may see. Some embodiments may include playing the video or a fraction of the video if a user hovers over the thumbnail. The videos, original images, and thumbnails are sent to the video and image cache where they may be accessed by a user in an application, web page, or another platform.

Segmentation labels are generated on each image and video either at the time of upload, while stored in the cache, or when accessed by a user. Phenotype predictions may be determined (such as heart rate, blood flow, and loss of sight) using optical flow, automatic comparisons, and Fourier Transform. These methods may be used to train models to make phenotype predictions. Other methods of training may be used such as implementing manual training data. These techniques may be employed individually or in tandem.

Once the images and videos include phenotype predictions and segmentation labels, the information is sent to an AWS Relational Database Service (RDS) to be compared with user annotations, experimental metadata, and chemical databases. In some embodiments, the users that upload the videos and images may input experimental and chemical data pertaining to the videos and images. Ex. If a certain chemical is used on a zebrafish, the image of the zebrafish is stored along with the name, ID, and other metadata pertaining to the chemical.

The comparison of the data is sent to a Flask API containing the database model, automated data imports, common data queries, and background job manager. The data is sorted into categories based on image type, video type, predicted phenotype, segmentation label, chemical used, gene data, or other experimental metadata. All of the created and collected data is sent to React JavaScript UI. The UI includes a plate viewer, a chemical search, an image-and-video viewer, a gene search, an experimental noted editor, and a data quality marking/modification system. APIs other than Flask and UX/UIs/GUIs/other than React JS may be used to fulfill similar purposes. Users such as stakeholders, data scientists, engineers, zebrafish technicians, or other users may access the UI to see all the data and may alter it to fit their needs. Certain users may have more permissions that allow them to alter data, and some data may be encrypted or hidden from certain users for security purposes.

It would be possible to create a phenotyping platform similar to the zebrafish phenotyping platform using other animals that share human orthologs. These may include mice, rats, fish, birds, other mammals, insects, or other animals. It would also be possible to create a similar platform with the use of plants to determine the causes of phenotypes based on image data.

### (18) Mounting of Zebrafish Using Stamps

In order to facilitate robust quantitative analysis, mounting methods have been developed to enable the simultaneous imaging of a greater number of embryos in near-to-identical orientations. This enables the acquisition of a sufficient amount of data in standardized and reproducible conditions for the observation and comparison of phenotypic traits over time using high-resolution microscopes, a method that is heavily relied upon in the field of developmental biology, particularly with the use of transparent model organisms such as Danio rerio (zebrafish), which can be easily observed and imaged using fluorescent proteins. The use of fluorescence is not, however, the only method of observing images using high-resolution microscopes. Some embodiments use bright field imaging coupled with machine learning methods to highlight elements of images without the need for alteration of genes or injecting fluorescent molecules.

Some embodiments include an improved mounting method that allows for semi-automated, high-content imaging of zebrafish embryos. This method utilizes a 3D-printed stamp to create a 2D coordinate system of wells in an agarose cast or other medium, which is modeled on the average morphology of zebrafish embryos from fertilization to about 96 hours post-fertilization. These molds allow for consistent and reproducible angles and can be easily and cheaply reproduced with a 3D printer. Molds can also be reused to create additional casts, which can be reused after imaging. These casts are compatible with any upright microscope and can be adapted for use on an inverted microscope, taking the working distance of the objective used into account

Cells from zebrafish embryos can be grown in either mixed or pure cultures from various stages of development to monitor the dynamics of transient cell populations, control the chemical and physical stimuli that the cells receive, and easily conduct chemical screenings. The zebrafish and zebrafish embryos being tested may be cultured in homogeneous or heterogeneous cultures.

Most imaging data is collected 22-48 hours post-fertilization, though this may depend on the target genome and phenotype of interest. This standardized and reproducible arrangement provides a custom well plate for the respective imaging software, enabling a semi-automated imaging process. The improved orientation also reduces post-processing, improves the comparability of volumetric data, and decreases light exposure, thereby reducing photo-bleaching, and photo-toxicity, and improving the signal-to-noise ratio. This method also enables high-content imaging of zebrafish embryos in a semi-automated, well-plate-like manner on inverted confocal microscopes. This improves the image data quality and acquisition efficiency, making it a useful method for those using the services of a microscopy facility.

Due to the advancement of high-resolution microscopes, image analysis methods, and computer technology, developmental biology is now undergoing a digital and quantitative transformation, which is essential in order to observe and understand biological processes. To gain a quantitative and representative view of such processes, it is important to record a sufficient amount of samples. To process biological samples of whole organisms, it is important to have a standardized way of sample mounting, data acquisition, data processing, and analysis. Thus, high-resolution imaging of multiple samples in standardized conditions has become a key element in developmental biology. Zebrafish have been established as a model organism for vertebrate development due to their genetic tractability and robustness, transparency, high number of offspring, and short development time, which make them ideal for live imaging and high-content screening. Some embodiments may maximize the benefits of these advantages by mounting, imaging, and analyzing numerous zebrafish embryos all at once and under similar conditions by using 3D stamps to align the zebrafish embryos in a predetermined pattern.

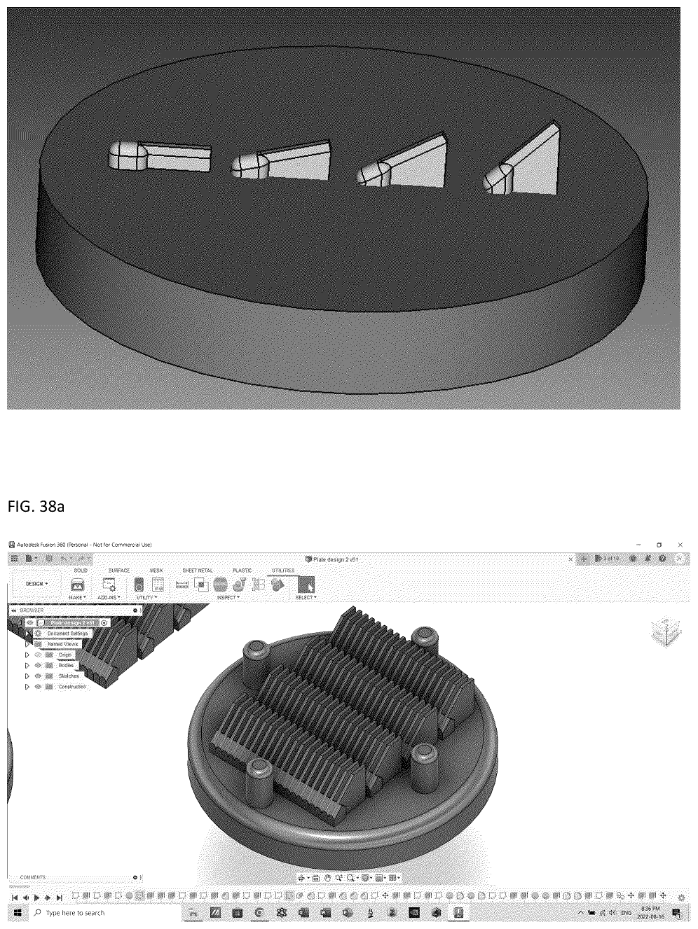

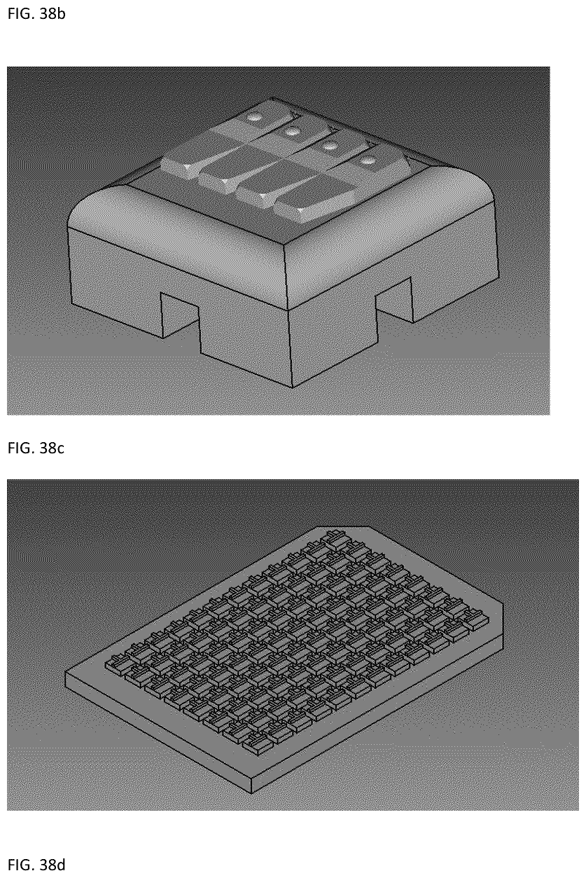

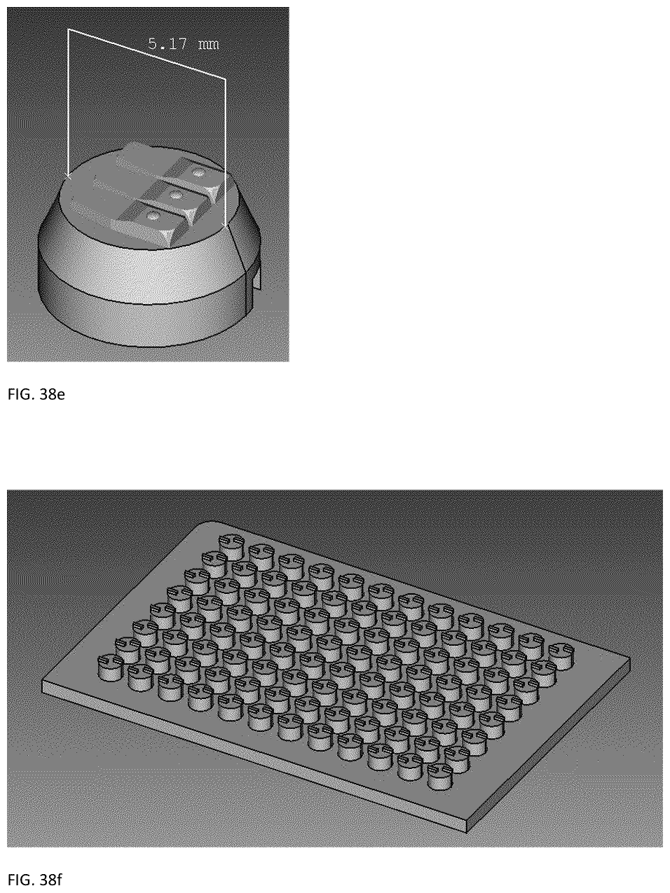

In order to evaluate and compare morphological phenotypes or reporter gene expression patterns in various assays, a focus was placed on developing tools to routinely generate agarose molds within wells of microtiter plates to hold and position both zebrafish embryos and larvae in their lateral and dorsal views. For flexibility and to minimize printing time, these tools were made up of a base plate and a set of stripes containing rows of pins, with a flattened cylindrical shape tapering to a point on two sides. This design takes into account the size and shape of embryo features like the yolk and trunk and is empirically optimized. It also replicates earlier designs: yolk-ball holding pins for lateral orientation, and keel shape designs to support dorsal orientation when using inverted microscopes. Deep cavities reduce the movement of the surrounding medium and improve the overall embryo positioning in the wells of microtiter plates. These features are used in the stamps shown in FIGS. 38c and 38e.

Some embodiments may include 1-phenyl 2-thiourea (PTU), which is a Tyr (tyrosinase) inhibitor that is used to block pigmentation and improve optical transparency in zebrafish embryos. This allows for better imaging of the internal organs of the zebrafish embryos using a non-invasive, non-destructive technique. This allows scientists or researchers to view the internal organs of the zebrafish without the need for sectioning organs. Certain embodiments may include imaging either live or not live zebrafish during and after in vivo testing. This may include freezing samples of important organs or whole zebrafish to perform further testing later if needed.

Some embodiments include creating a 3D stamp to align zebrafish embryos in a single-well or multi-well plate. These stamps allow for pictures of zebrafish to be taken in a matrix formation to allow for a better comparison of zebrafish using a single image. A preferred embodiment includes using the stamp to indent angled protrusions into an agar base to create a matrix of cavities before the development of the zebrafish. A single zebrafish embryo may be placed in each cavity to allow a matrix of zebrafish to develop in the same orientation.

The angle of these protrusions may depend on the orientation of the zebrafish needed during experimentation. Certain angles may have a better view of certain organs. This may allow for a better look at an organ being tested for a phenotype of interest. For example, certain angles may allow for a better ventral view of zebrafish embryos. This would allow for a better look at the heart of individual zebrafish. In some embodiments, these images are used/uploaded to the zebrafish phenotyping platform to allow for the prediction of phenotypes and automatic labeling of organs.

FIG. 38a contains an image of tested angles used for experimentation. These specific angles were tested at 0, 15, 30, and 45 degrees to allow for ventral view testing of zebrafish. After this testing was performed, a stamp with 80 protrusions at the determined angle was created to create 80 wells in a single plate containing agar. Some embodiments may include a plate containing substances other than agar or a combination of agar and other substances. This 80-protrusion plate is shown in FIG. 38b. In some embodiments, the stamps are 3D printed to produce maximum uniformity on each protrusion while creating the stamps faster than other methods such as creating the stamps by hand. It is possible to create stamps through other methods (such as by hand), and these methods may have advantages over 3D printing. Other stamps and angles may also be created to allow for better views of zebrafish during and after experimentation/imaging.

FIGS. 38e and 38f contain models of low-resolution stamps that may also be used to create matrices of particular views of zebrafish. These low-resolution stamps may be adapted to any high-resolution stamp such as the addition of yolk-ball holding pins on each protrusion.

These stamps may be used to create different orientations of zebrafish embryos for imaging. Some embodiments may contain a single stamp that provides different views of zebrafish. These embodiments may allow for multiple views in a single image of similar zebrafish. Preferred embodiments contain stamps that align all zebrafish in a single orientation for imaging. Certain alignments may allow better images of certain organs and features of the zebrafish being imaged. This alignment allows for better comparisons of organs that may slightly differ between zebrafish.

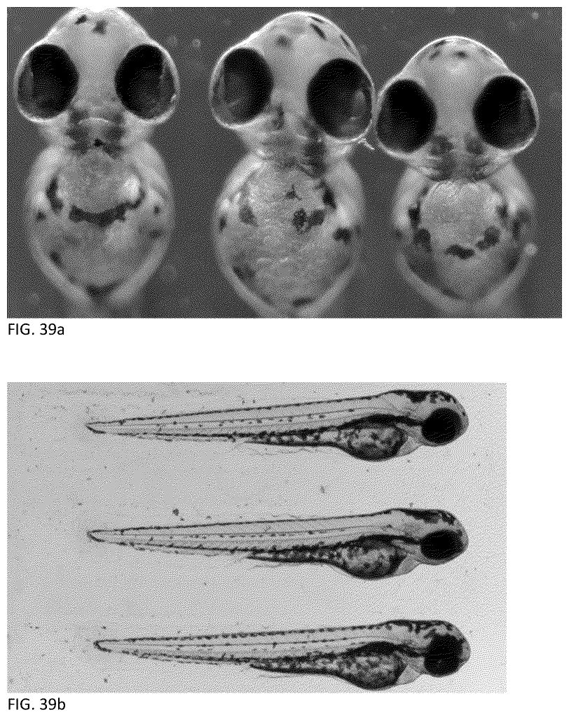

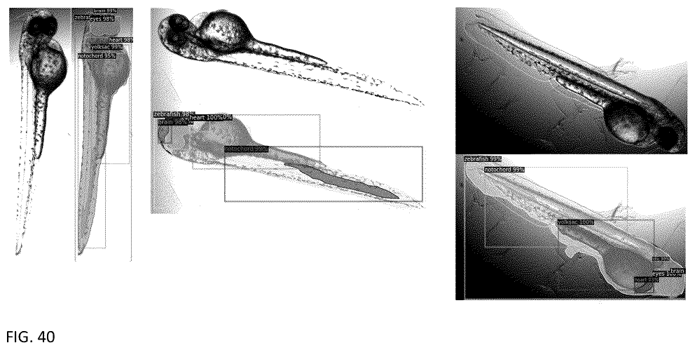

FIG. 39a is a frontal view (or ventral view) of zebrafish. This view allows for a better view of the heart, eyes, and face. FIG. 39b is a lateral view of zebrafish. This view allows for a better view of the tail and blood flow. Other alignments may allow other organs and features of interest to be seen during imaging. These views were made possible by stamping each plate and inserting zebrafish embryos into each divot created by the stamp.

The stamps are created to help align zebrafish in a certain orientation, but zebrafish are living creatures that may move around and become unaligned. After using the stamps, some zebrafish may need to be re-aligned. These zebrafish may be manually re-aligned or automatically re-aligned to provide better imaging in a certain view and to keep all zebrafish in the same orientation for comparison purposes.

### (19) Organ Segmantation, Optical Flow, and Fourier Transform

Organ segmentation is the process where an input image is automatically annotated by a machine learning algorithm to segment regions of interest. Unlike bounding box predictions that are only characterized by a rectangular outline, segmentation identifies a high-resolution bounding polygon for each detected object. These bounding polygons may be highlighted and detected using ML models. These polygons may represent the shape of the specific organ of the zebrafish being segmented.

Some embodiments may include the use of bounding box predictions to characterize specific organs in a rectangular outline. These outlines may allow users to view organs without the need for highlighting. This may preserve the color of the organ and may allow for easier viewing of overlapping organs. This may also be useful when dealing with organ systems. For example, a bounding box may detect the cardiovascular system while organ segmentation is used to highlight the heart as part of the system.

Organ segmentation is achieved via manually annotating a large number of zebrafish image and videos using the LabelMe software with regions of interest, fine-tuning a Detectron2 model (pretrained on COCO), and applying predictions on unseen data to identify specific organ regions and capture downstream metrics (e.g. heart rate, organ size, organ shape, etc.) This training data may pertain to annotated images and video data from a database or from users. The models may also be updated based on annotations made by users in the zebrafish phenotyping platform.

FIG. 40 contains multiple images of the automated segmented labeling created automatically by a machine learning model. The models are trained to locate organs based on multiple views, and they may be manually corrected if necessary. The elements of interest are highlighted in various colors to distinguish elements from each other. These elements may pertain to organs, organ systems, blood, tissues, or other elements such as yolk-sack or tail.

When paired with the zebrafish phenotyping platform, a user may locate specific organs of interest and highlight them in any color they wish. Some embodiments include uploading multiple images and using the ML models to compare the various sizes, shapes, and placements of organs of interest. Some images may include multiple zebrafish per image. In these embodiments, the ML models can detect and display target elements for each zebrafish in the image.

In some embodiments, heart regions may be captured using the trained organ segmentation model above instead of via optical flow strategies. This ensures that even in hearts with little or irregular movement, heart rate can be captured accurately. Signals are calculated as the average pixel intensity across the entire pericardium area, these are then smoothed using a 1-D Gaussian filter. From here, all peaks are found using standard peak-picking algorithms. The frequency of these peaks is used to identify beats per minute as well as other high-resolution organ features. Other software for peak picking, signaling, signal calculation, and signal filtering may also be used.

Certain embodiments may focus on the heart region of the zebrafish. The heart region may be detected via organ segmentation, optical flow, or a mixture. Certain embodiments may use optical flow when heart movement is regular and consistent. In other embodiments, heart regions are captured using a trained organ segmentation model, signal data, and peak-picking algorithms instead of via optical flow strategies. This ensures that even in hearts with little or irregular movement, heart rate can still be captured accurately.

Beyond heart rate, the organ segmentation model is used to capture several important features including heart chamber size, systolic and diastolic diameters, heart period, contraction wave velocity, cardiac arrhythmicity, stroke volume, edema, etc.

Embodiments below include methods of capturing elements using optical flow. These embodiments are especially relevant for the capturing of moving elements such as blood or heart movement.

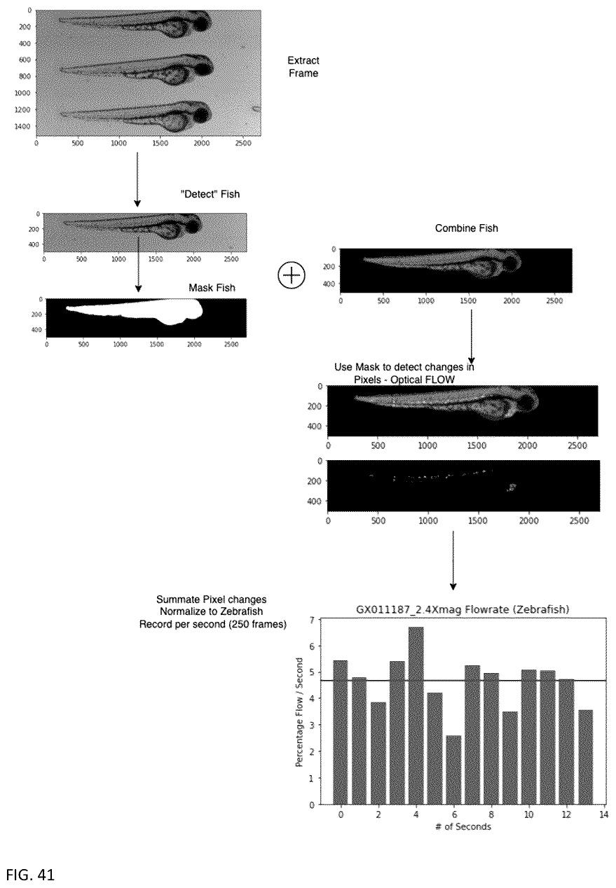

FIG. 41 is a flowchart for the creation of an optical flow pattern and video. Videos are extracted frame by frame, and a machine-learning algorithm is used to detect the outline of the zebrafish in each frame. A “mask” is created where the outline of the fish is removed from each frame and added to a background. The zebrafish and the background are combined into a single image (or video) and ML algorithms are used to detect changes in each frame. This allows for movement (or optical flow) in the videos to be captured and highlighted. The new frames are compiled into a single video that includes only movement. This is useful when determining blood flow, heart rate, etc. and this information may be used to detect changes in phenotypes. This specific embodiment includes 250 frames per second, but any number of frames per second may be used in other embodiments. The graph below pertains to the normalized pixel changes over time in the video. This normalization may be used to create still images of areas that include more movement over time. This helps to distinguish slight movements of the fish in certain frames but allows for the detection of areas of higher movement. In some embodiments, these areas of high movement may be highlighted in a contrasting color for users to see easily. The movement may also be seen overlayed with the image of the zebrafish to allow users to go back and forth between the image of the zebrafish and the image without the zebrafish. The pixel changes are summated and averaged per pixel over time to allow the pixels with the most changes to be detected and recorded. These pixels may include movement areas of interest.

In some embodiments, the pixels are normalized to speed up the model learning. Pixel normalization is a process in which the values of individual pixels in digital images are adjusted to create a common set of values across the entirety of the image. It is used to minimize variations in pixel values caused by different lighting and exposure conditions, as well as to make individual colors more uniform across an image. Pixel normalization can be used to enhance the contrast of an image, reduce noise, and improve the overall visual quality of the image.

Data Analysis and Fourier transform may be used in combination with optical flow and/or false color tracking. Many functions that occur in nature can be broken down into a sum of other “basis” functions. One such set of basis functions is cosine and sine of various frequencies. These are especially useful when the original function (typically called a ‘signal’ in this context) is oscillatory. This process of converting a raw signal into a series of basis functions is called the Fourier transform.

Consider that a zebrafish video consists of K still images (frames) that are N pixels in height, and M pixels in width. If a fixed pixel is present, then one can treat the brightness of the pixel as a discrete signal as the frames change. When the pixel is bright, the signal's value is large. When the pixel is dark, the signal's value is small. This can be done for all pixels in the video, yielding N×M discrete signals.

Since the heart of the zebrafish beats with fairly consistent oscillations, it's reasonable to assume that a pixel that is in an area corresponding to the heart region (or any other blood vessel) of the fish should experience some oscillatory fluctuations in its brightness. The Fourier transform analysis enables oscillating patterns to be identified in each zebrafish video, giving informative features on the zebrafish phenotype and response to both chemical and genetic perturbations.

When paired with the zebrafish phenotyping platform, a user may highlight the optical flow of any video to determine areas of high movement in the zebrafish. Some images and videos may contain multiple zebrafish. In these images, the optical flow may be determined on all zebrafish in the image or video or determined on an individual basis.

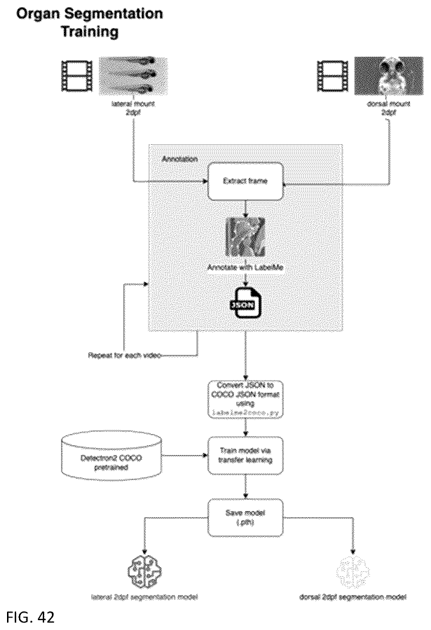

FIG. 42 is a flowchart depicting ML training methods used for the Automated Segmentation of zebrafish images. In the figure, the videos pertain to lateral and dorsal mounts, though the process would be the same for any view of the zebrafish. Video or images are extracted from a database or uploaded by a user to the platform. Images or singular frames are annotated with LabelMe. Some embodiments may include annotations being performed with other annotation/labeling software. Each video/image in the training set is annotated and then all of the singular frames with annotations are converted to JSON. Other formats are possible, but JSON allows for easy adaptability for APIs and UIs. The JSON files are converted to COCO JSON using a converting software. A pretrained model is trained with the files using transfer learning. Other embodiments may include a model that has not been pretrained to provide for more configurability of the model, though this would take more time than using pretrained model. The model, now trained with the annotated frames, is saved and may be used on the zebrafish phenotyping platform to highlight elements of user uploaded images and videos.

The annotations performed may include labels given to organs, tissues, and organ systems of each zebrafish. The ML model may also be trained to detect multiple zebrafish per frame. These labels include but are not limited to, the whole zebrafish, the eye(s), the yolk sac, the pericardium, the ventricle, the atrium, the notochord, the myotome, the fin fold, the head, the otolith, the heart, the blood vessels, the liver, the brain, the different lobes of the brain, the different lobes/ventricles of the liver, the different lobes/ventricles of the heart, the veins, the arteries, etc.

Other embodiments may include training models using images containing other organisms or bacteria. This may allow for the detection of various organs, tissues, and organ systems in images using trained ML models. Once detected, these organs, tissues, and organ systems may be highlighted to allow users to easily detect various changes in phenotype or these organisms. These trained models may be used in combination with another platform for phenotyping these other organisms or bacteria.

Certain embodiments may also include training an ML model to determine the age of the embryo/zebrafish based on developmental characteristics. This is possible because of the fast development rate of zebrafish. For example, zebrafish embryos already exhibit beating hearts 24 hours post-fertilization. This may be observed either manually or using computer software.

What is claimed is:

1. A platform for zebrafish phenotyping comprising:
a user;
image and video data containing images and videos containing at least one zebrafish and elements pertaining to the zebrafish;
a first set of machine learning models used to predict zebrafish phenotypes based on the image and video data;
a second set of machine learning models used to automatically detect and highlight the elements pertaining to the zebrafish in one of the images and videos containing zebrafish.

2. The platform of claim 1, further comprising trained machine learning models used to provide relationship data between phenotypes and genetic data.

3. The platform of claim 1, wherein the trained machine learning models are used to predict zebrafish phenotypes.

4. The platform of claim 1, wherein the detected elements pertain to at least one of organs, organ systems, tissues, whole organisms, and other biological elements.

5. The platform of claim 1, wherein the at least one of images and videos contains more than one zebrafish.

6. The platform of claim 5, wherein the elements are detected in each zebrafish in the one of images and videos.

7. The platform of claim 1, wherein the detected elements pertain to an abnormal phenotype

8. The platform of claim 7, wherein the abnormal phenotype is one of an enlarged heart, loss of sight, disrupted blood flow, and loss of an organ.

9. The platform of claim 1, wherein the user is one of a researcher, scientist, engineer, investor, and developer.

10. The platform of claim 1, wherein at least one of the first set and second set of machine learning models trained using training data.

11. The platform of claim 10, wherein the training data includes images and videos containing zebrafish and labeled elements pertaining to the zebrafish.

12. The platform of claim 11, wherein the labeled elements pertain to at least one of organs, organ systems, tissues, whole organisms, and other biological elements.

13. The platform of claim 1, wherein the first set of machine learning models and the second set of machine learning models pertain to the same machine learning models.

14. The platform of claim 1, wherein the first set of machine learning models and the second set of machine learning models pertain to different machine learning models.

15. The platform of claim 1, wherein the zebrafish in the images and videos are aligned in a specific orientation.

16. The platform of claim 15, wherein the specific orientation is one of a lateral view, frontal view, ventral view, and dorsal view.

17. A method for zebrafish phenotyping comprising:
a platform comprising;
a user;
image and video data containing at least one of images and videos containing at least one zebrafish;
trained machine learning models used to predict zebrafish phenotypes based on the image and video data;
trained machine learning models used to detect elements in one of the images and videos containing zebrafish;
using the platform to determine the phenotypes of zebrafish based on the image and video data in the platform.
